(19) United States
(12) Patent Application Publication
(10) Pub. No.: US 2025/0311973 A1
(43) Pub. Date: Oct. 9, 2025

(54) SYSTEMS AND METHODS FOR PROVIDING THERAPY MANAGEMENT GUIDANCE TO PATIENTS TO OPTIMIZE GLP-1 THERAPY EFFECTIVENESS

(71) Applicant: Dexcom, Inc., San Diego, CA (US)

(72) Inventors: Matthew L. JOHNSON, San Diego, CA (US); Qi AN, San Diego, CA (US); Rush BARTLETT, Austin, TX (US); John PADERI, San Francisco, CA (US)

(21) Appl. No.: 19/174,862

(22) Filed: Apr. 9, 2025

Related U.S. Application Data
(60) Provisional application No. 63/631,971, filed on Apr. 9, 2024.
(60) Provisional application No. 63/734,516, filed on Dec. 16, 2024.

(51) Int. Cl. A61B 5/00; A61B 5/145; A61B 5/1486; G16H 10/60; G16H 20/10

(52) U.S. Cl. CPC A61B 5/4848; A61B 5/0004; A61B 5/002; A61B 5/14532; A61B 5/14546; A61B 5/14865; A61B 5/4238; A61B 5/4839; A61B 5/4866; A61B 5/7278; G16H 10/60; G16H 20/10

(57) ABSTRACT

Certain aspects of the present disclosure provide a monitoring system comprising a continuous analyte sensor configured to penetrate a skin of a patient and generate a sensor current indicative of analyte levels of the patient, and a sensor electronics module coupled to the continuous analyte sensor. The sensor electronics module comprises an analog to digital converter configured to receive the sensor current and convert the sensor current generated by the continuous analyte sensor into digital signals, one or more processors configured to convert the digital signals to a set of analyte measurements indicative of the analyte levels of the patient, and a Bluetooth antenna configured to transmit the set of analyte measurements wirelessly to a wireless communications device using Bluetooth or BLE communications protocols.

### CROSS-REFERENCE TO RELATED APPLICATIONS

This application claims priority to and benefit of U.S. Provisional Application No. 63/734,516, filed Dec. 16, 2024, and U.S. Provisional Application No. 63/631,971, filed Apr. 9, 2024, which are hereby assigned to the assignee hereof and hereby expressly incorporated by reference in their entirety as if fully set forth below and for all applicable purposes.

### INTRODUCTION

GLP-1 medications have been known to be effective for diabetic patients to control blood sugar levels, as GLP-1 drugs mimic the action of glucagon-like peptide hormone and stimulate the body to produce insulin after a meal. GLP-1 medications have also been prescribed to patients whose health would benefit from weight loss. GLP-1 medications are now some of the most popular medications for weight loss. However, GLP-1 medications can cause negative side effects, causing the patient to stop taking the medication and/or become non-compliant with their prescribed dose and/or frequency. Even further, once a patient reaches their weight loss goals and begins to titrate down and/or completely stops taking GLP-1 medications, most patients regain the weight without proper weight management.

### BRIEF DESCRIPTION OF THE DRAWINGS

So that the manner in which the present disclosure can be understood in detail, a more particular description may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example therapy management system used in connection with implementing embodiments of the present disclosure.

FIG. 2 is a diagram conceptually illustrating an example continuous analyte monitoring system including example continuous analyte sensor(s) with sensor electronics, according to certain embodiments of the present disclosure.

FIG. 3 illustrates example inputs and example metrics that are calculated based on the inputs for use by the therapy management system of FIG. 1, according to certain embodiments of the present disclosure.

FIG. 4A illustrates an example method for providing therapy management guidance to optimize a GLP-1 regimen in order to minimize and manage gastrointestinal symptoms caused by GLP-1 medication, according to certain embodiments of the present disclosure.

FIG. 4B illustrates an example method for providing therapy management guidance to optimize a GLP-1 regimen in order to minimize and manage gastrointestinal symptoms caused by GLP-1 medication, according to certain embodiments of the present disclosure.

FIG. 5A illustrates an example method for providing therapy management guidance to optimize a GLP-1 regimen for weight loss and to maintain weight loss over time, according to certain embodiments of the present disclosure.

FIG. 5B illustrates an example method for providing therapy management guidance to optimize a GLP-1 regimen for weight loss, according to certain embodiments of the present disclosure.

FIG. 6 illustrates an example method for providing therapy management guidance for maintaining weight loss over time, according to certain embodiments of the present disclosure.

FIG. 7 is a flow diagram depicting a method for training machine learning models to predict gastrointestinal symptoms, an expected weight loss of a patient, and/or provide guidance for medication parameters, specific diet recommendations, and/or exercise regimens to minimize symptoms, optimize weight loss, and/or maintain weight loss, according to certain embodiments of the present disclosure.

FIG. 8 is a block diagram depicting a computing device configured to perform the operations of FIGS. 4, 5, and 6, according to certain embodiments of the present disclosure.

FIGS. 9A-9B depict exemplary enzyme domain configurations for a continuous multi-analyte sensor, according to certain embodiments of the present disclosure.

FIGS. 9C-9D depict exemplary enzyme domain configurations for a continuous multi-analyte sensor, according to certain embodiments of the present disclosure.

FIGS. 10A-10B depict alternative views of an exemplary dual electrode enzyme domain configuration for a continuous multi-analyte sensor, according to certain embodiments of the present disclosure.

FIGS. 10C-10D depict alternative views of an exemplary dual electrode enzyme domain configuration for a continuous multi-analyte sensor, according to certain embodiments of the present disclosure.

FIG. 10E depicts an exemplary dual electrode configuration for a continuous multi-analyte sensor, according to certain embodiments of the present disclosure.

FIG. 11 is an illustration of an example planar analyte sensor with sensing membranes, according to certain embodiments of the present disclosure.

FIGS. 12A-12E illustrate a double-sided, co-planar un-connected analyte sensor, according to certain embodiments of the present disclosure.

FIGS. 13A-13E illustrate a double-sided, co-planar connected analyte sensor, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

GLP-1 medications have become increasingly popular for treating various health conditions including kidney disease, liver disease, diabetes, obesity, and other conditions in which weight loss is desired. Regardless of the popularity of GLP-1 medications, existing techniques for determining the effectiveness of a GLP-1 dose rely on point-in-time measurements. For example, because, GLP-1 medications lead to weight loss, one way to measure the success of the medication regimen is to monitor the resulting weight loss. Currently, a patient's weight is usually monitored using point-in-time measurements such as body weight measurements obtained every few weeks or months.

However, in most cases, the GLP-1 medications take weeks to show positive or negative changes. Therefore, it could take many weeks for a health care provider or the patient to determine the effectiveness of the prescribed dosage. In the meantime, because in many cases, GLP-1 medications cause negative side effects, the patient can become discouraged and become non-compliant.

In addition, even if the patient continues to take the GLP-1 medication, the manner in which they are losing weight (e.g., fat vs. muscle loss) can have drastic effects on their disease management, as well as the future prognosis of their health. For example, most patients have to stop taking GLP-1 medications due to nutritional deficiency or reaching their desired health or weight goals. However, if not managed properly, during treatment or after treatment, patients can experience negative effects after stopping their medication regimen. For example, if the patient experiences fat gain rather than muscle gain after stopping their medication regimen, the additional fat gain causes them to be worse off than when they began taking the GLP-1 medication.

Further, one of the biggest negative side effects of GLP-1 medications is that they effect the gastrointestinal tract. Current techniques for monitoring and identifying negative gastrointestinal symptoms caused by GLP-1 medications are limited and revolve around symptom-management, which is often based on patient reported symptoms at a single point in time.

Examples of negative gastrointestinal symptoms caused by GLP-1 medications can include upper and lower gastrointestinal symptoms including gastroparesis, bloating, nausea, vomiting, acid reflux, diarrhea, etc. In certain cases, if one or more of these symptoms are left untreated, the patient can develop more severe health complication over time such as thyroid cancers, pancreatitis, and/or gall bladder disorders. Negative gastrointestinal symptoms and health complications can be a dose dependent response to the amount of GLP-1 medication the patient is prescribed and can be determined based on a patient's gastric emptying.

As described herein, gastric emptying refers to the process by which the stomach empties material into the small intestine. The rate of gastric emptying refers to the speed at which the stomach empties the material into the small intestine. Therefore, a decrease in the rate of gastric emptying refers to a decrease in the speed at which gastric material is cleared from the patient's stomach following a meal. As described herein, particular analyte metrics or behavior can be indicative of or directly correlate to the rate of gastric emptying and/or a change therein. As such, for example, a rate of change in analyte levels, such as glucose levels or lactate levels, are used in certain embodiments herein as a proxy for, or used to derive a rate of gastric emptying. Thus, as described in further detail below, the embodiments herein provide a technical solution by determining the rate of gastric emptying of the patient based on the rate of increase of analyte levels and/or other analyte metrics.

When administering GLP-1 medication, it is important to determine the correct regimen, balancing the negative side effects of GLP-1 medications with the effectiveness of the GLP-1 dosage. If the GLP-1 dose and/or frequency is too high, the patient can experience a decrease in the rate of gastric emptying to an extreme level (e.g., slowing and/or completely stopping movement of food from the stomach to the small intestine), which is known as gastroparesis, or other gastrointestinal symptoms. Alternatively, if the GLP-1 dose and/or frequency is too low, the patient will not experience their desired weight loss. In addition to GLP-1 dose or frequency, behavioral factors such as the diet of the patient and the activity level of the patient can effect the severity or presence of such gastrointestinal symptoms.

As described above, single point in time assessments of negative gastrointestinal symptoms that are based on patient reported symptoms, are likely inaccurate, subjective, and prone to error, and therefore not optimal for purposes of determining the optimal GLP-1 dose and/or adjusting behavioral factors that influence the side effects of the medication. For example, single point in time patient reported symptoms can be affected by confounding factors that the patient has failed to report or the physician has not accounted for. Additionally, single point in time symptom assessment techniques do not allow for predicting or determining when a prescribed GLP-1 dose is too high and likely to cause symptoms in the future, or when the GLP-1 dose is optimal but causing gastrointestinal symptoms that are likely to resolve in a short period of time. In addition, these point in time measurements do not always take into account the behavioral factors such as diet and activity of the patient, as behavioral factors are not reported and not easy to monitor by a physician.

As such, current methods for determining the effectiveness of GLP-1 medications face many challenges in efficiently and accurately determining the effect of a GLP-1 dose for a patient. Consequently, there is a need in the art for an accurate, continuous solution to monitor a patient on GLP-1 medication to optimize and sustain the positive effects of GLP-1 medications, maintain proper nutrition and weight loss, monitor for the presence or development of negative symptoms, and/or encourage compliance in real time.

Accordingly, certain embodiments described herein provide a technical solution to the technical problems described above by providing a continuous analyte monitoring system, including, one or any combination of a continuous glucose sensor, a continuous lactate sensor, a continuous ketone sensor, a continuous glycerol sensor, a continuous free fatty acid sensor, or a continuous amino acid sensor for use in determining the effectiveness of a GLP-1 regimen and/or for minimizing negative side effects of GLP-1 medication.

In particular, present disclosure relates generally to methods and systems for continuously monitoring analyte data, including one or any combination of glucose, lactate, ketones, glycerol, amino acids, or free fatty acid levels, and/or non-analyte data to optimize GLP-1 regimen effectiveness. Aspects of the present disclosure utilize analyte data, and can further utilize non-analyte data of a patient, to determine whether the patient is achieving their weight loss goal on their current GLP-1 regimen. Upon determining if the regimen is optimal, i.e., the regimen achieves the intended effect while minimizing the negative side effects, aspects of the present disclosure further provide patient-specific therapy management guidance (e.g., regarding meal times, optimal diet recommendations, medication recommendations, and/or lifestyle changes (e.g., maintain a specific exercise regimen, etc.)) to maximize effectiveness of a GLP-1 regimen while encouraging medication compliance and minimizing the development of negative side effects. These negative side effects can include gastrointestinal symptoms.

Continuous analyte measurements, as proposed herein, provide a more accurate indication of the effect of a GLP-1 medication for health management over time as compared to a single point in time measurement. A single point in time reading can be influenced by a patient's diet or activity changes near or during the point in time and/or does not demonstrate changes until the patient has been taking the GLP-1 medication for an extended period, such as 2 months. Additionally, continuous analyte measurements, as proposed herein, provide more accurate monitoring of a patient on a GLP-1 medication to predict and/or reduce negative side effects such as gastrointestinal symptoms and/or health complications that can arise from an incorrect or ineffective GLP-1 regimen for the patient. As described herein, the GLP-1 regimen of the patient refers to a dose of the GLP-1 medication, a timing of the GLP-1 medication administration, a frequency of GLP-1 medication administration, and/or a type of GLP-1 medication. Particularly, many types of GLP-1 medications exist (e.g., exenatide, liraglutide, dulaglutide, semaglutide, lixisenatide, etc.) and these types of GLP-1 medications can be taken at various doses, various times, and various frequencies based on the patient's response to GLP-1 medications as described herein.

Note that although certain embodiments described herein are described in relation to GLP-1 medications, the present disclosure can, additionally or alternatively, be configured to optimize other medications which can be prescribed for weight loss including glucose-dependent insulinotropic polypeptide (GIP), glucagon (GCG) receptors, etc. In addition, as GLP-1 medications are used for various health conditions, management of the efficiency of the GLP-1 medication can be related to other health outcomes (e.g., fat loss, muscle to fat ratio, liver health improvement, metabolic health improvement, kidney health improvement, glucose clearance improvement, improvement in insulin resistance, or other improvements in health that can be achieved by improving the overall health, metabolic health or glycemic control of the patient). In such examples, the GLP-1 medications for other health conditions can be managed with similar techniques to those described herein with respect to weight loss. Weight loss is used as an example of an outcome that is monitored with respect to GLP-1 medications. Similarly, while the present disclosure mainly discusses gastrointestinal symptoms as the negative side effect, other negative side effects such as nausea, vomiting, and similar discomfort or side effects can be managed using the methods described herein.

As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insight to be derived using the algorithms described herein for optimizing a GLP-1 regimen for weight loss or prevention/minimization of gastrointestinal symptoms. In other words, single point-in-time measurements collected as a result of a patient visiting their health care professional every few weeks or months results in sporadic data points (e.g., that are, at best, months apart in timing) that cannot form the basis of any meaningful data or insight to be derived. As such, without the continuous analyte monitoring system of the embodiments herein, it is simply impossible to continuously optimize weight loss, sustain weight loss, monitor for the presence or development of negative gastrointestinal symptoms, and encourage compliance for patients on GLP-1 medications, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for providing real-time optimization of GLP-1 regimen for weight loss while minimizing negative gastrointestinal symptoms, as well as real time therapy management guidance to maintain positive effects of the GLP-1 medication when the patient stops and/or begins decreasing a GLP-1 dose or a GLP-1 frequency. As used herein, positive effects of the GLP-1 medication can refer to weight loss, fat loss, metabolic health improvement, kidney health improvement, liver health improvement, or other health-related improvements that can be achieved by improving the overall health, metabolic health, or glycemic control of the patient.

Real time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least glucose and/or other analyte concentration values using calibration techniques described herein to account for the sensitivity of the continuous analyte sensor(s), and (3) transmitting measured glucose and/or analyte concentration data, including lactate and/or other analyte concentration values, to a display device via wireless connection.

For example, the at least one sensor electronics module can be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured lactate and/or other analyte concentration data to a display device at a particular transmission period (or rate), which can be the same as (or longer than) the sampling period, such as every 1 minute (0.016 Hz), 5 minutes, 10 minutes, etc.

The real-time analyte data that is continuously generated by the continuous analyte monitoring system described herein, therefore, allows the therapy management system herein to provide real-time optimization of GLP-1 regimen for weight loss or one or more other positive effects of the GLP-1 medication while minimizing negative side effects, e.g., gastrointestinal symptoms. The therapy management system can also provide real time therapy management guidance to maintain the weight loss or one or more other positive effects of the GLP-1 medication when the patient stops and/or begins decreasing the GLP-1 dose or the GLP-1 frequency, which is technically impossible to perform using existing or conventional techniques or systems. Further, because of the real-time nature of this data, it is improbable that a human can manually and/or mentally continuously process a real-time data stream of analyte values over time to derive meaningful data and insight using the algorithms and systems described herein to optimize GLP-1 regimen while minimizing negative side effects, as well as provide real time therapy management guidance to maintain the positive effects of the GLP-1 medication when the patient stops and/or begins titrating down the GLP-1 dose or the GLP-1 frequency.

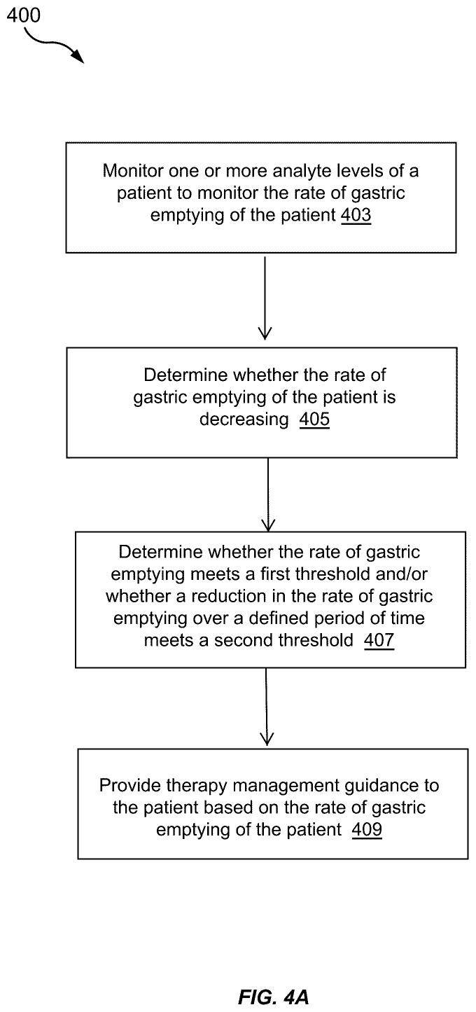

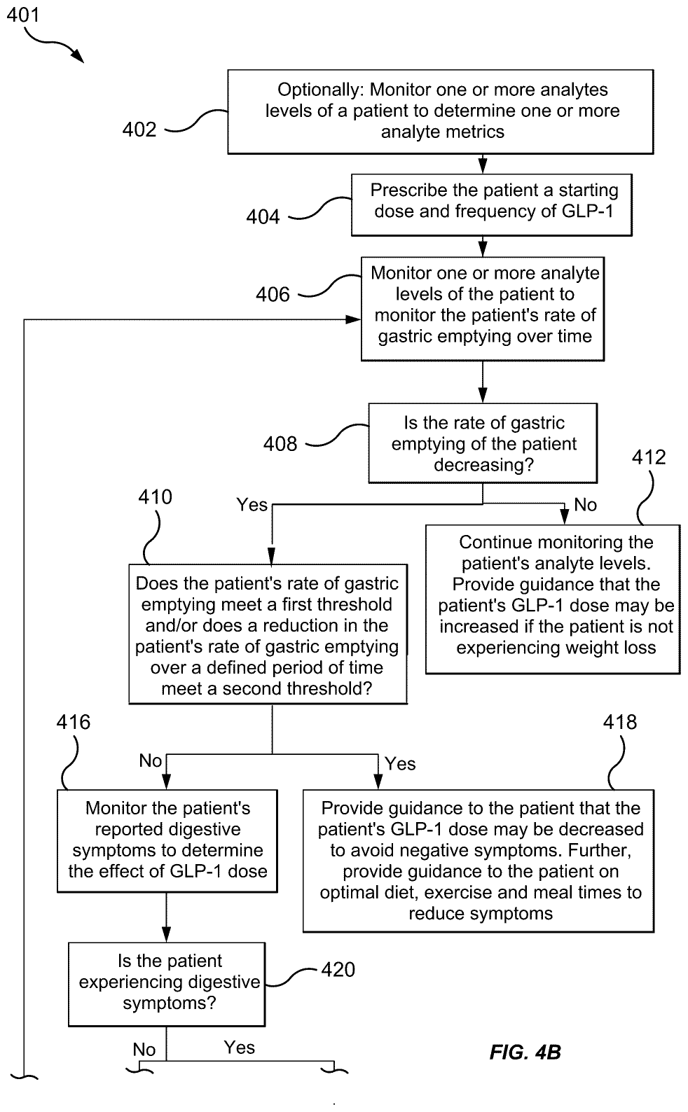

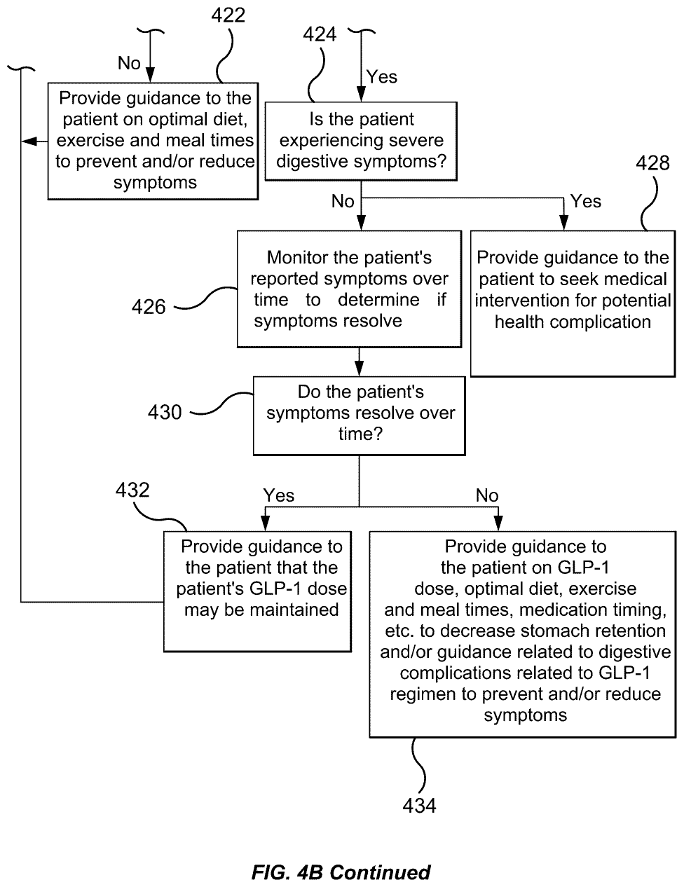

In other words, deriving meaningful data and insight from a stream of real-time data that is continuously generated, processed, calibrated, and analyzed, using the algorithms and systems described herein, is not a task that can be mentally performed. For example, executing the algorithms described in relation to FIGS. 4-7 in real-time and on a continuous basis, which would involve using a stream of real-time data that is continuously generated by a host's continuous analyte monitoring system and/or significantly large amount of population data (e.g., hundreds or thousands of data points for each one of thousands or millions of patients in the patient population) is not a task that can be mentally performed, especially in real-time.

Further, certain embodiments herein are directed to a technical solution to a technical problem associated with analyte sensor systems. For example, the therapy management system described herein maximizes an effectiveness of a patient's GLP-1 regimen to optimize the weight loss and/or one or more other positive effects of the GLP-1 medication while minimizing negative side effects, and provides therapy management guidance in view of the GLP-1 regimen optimization, where such therapy management guidance includes automatically implementing one or more device settings (e.g., thresholds, diet and exercise schedules, etc.) within the therapy management system. In this way, adjustments to the therapy management system settings by the patient can be minimized, which also minimizes device hardware computation and/or network load requirements associated with those adjustments. When this process is implemented for a large group of patients, automatic optimization of GLP-1 regimen and therapy management guidance will significantly reduce network and/or computation requirements for the group, thereby improving performance of the one or more hardware computing systems implementing such therapy management systems.

Further, by accurately determining a patient's optimal GLP-1 regimen using the analyte monitoring system and providing therapy management guidance (e.g., medication parameters and/or meal or exercise recommendations) based on such determination, an accuracy of such therapy management guidance can be improved. This improved accuracy can, in turn, improve medication dosing instructions (e.g., dosing instructions sent to a hardware medicament pump) as well as meal or exercise recommendations sent to the patient by the therapy management system. Improved recommendations (such as diet, exercise, and medication recommendations) provided by the therapy management system can be followed by the patient, resulting in a favorable improvement of the patient's analyte data and overall health.

Additionally, as analyte data of the patient is continuously received over time, the therapy management system can identify the results of earlier therapy management guidance (both for a current patient as well as other patients sharing one or more characteristics with the current patient) and can continually refine future therapy management guidance for the current patient and other related patients based at least in part on these results. The continuous refinement of future therapy management guidance can improve the accuracy of guidance generated by the therapy management system for all patients.

Additionally, each analyte sensor system that is manufactured by a sensor manufacturer might perform slightly differently. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current can be expressed in units of picoAmps (pA) or counts. The amount of measured analyte can be expressed as a concentration level in units of milligrams per deciliter (mg/dL), and the calibration sensitivity can be expressed in units of pA/(mg/dL) or counts/(mg/dL). The calibration baseline refers to the amount of electrical current produced by the analyte sensor when no analyte is detected, and can be expressed in units of pA or counts.

The calibration sensitivity, calibration baseline, and other information related to the sensitivity profile for the analyte sensor system can be programmed into the sensor electronics module of the analyte sensor system during the manufacturing process, and then used to convert the analyte sensor electrical signals into measured analyte concentration levels. For example, the calibration slope (calibration sensitivity) can be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels can be determined using a sensitivity function M(t) that is based on the initial in vivo sensitivity (M₀) and the final in vivo sensitivity (M_{f}). The sensitivity function M(t) can be expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$ACL = count / M ( {t}_{i} ) \qquad Eq . 1$$

A calibration baseline (baseline) can also be used to determine a measured analyte concentration level (ACL) from an analyte sensor count value (count) at a time tᵢ, and Equation 2 presents one technique:

$$ACL = ( count - baseline ) / M ( {t}_{i} ) \qquad Eq . 2$$

In some embodiments, data collected while providing therapy management guidance to the patient can be used to further optimize the calibration of the data, both for the specific patient, and/or a population of patients. Data collected while providing therapy management guidance to the patient can further optimize the accuracy of the device and measurements provided by the device. The improvements to the accuracy of the device and the measurements can in turn optimize the data used to generate future measurements and/or therapy management guidance to patients.

### Example Therapy Management System Including an Example Analyte Sensor for Providing Therapy Management Guidance to Patients for Optimizing GLP-1 Medication Effectiveness

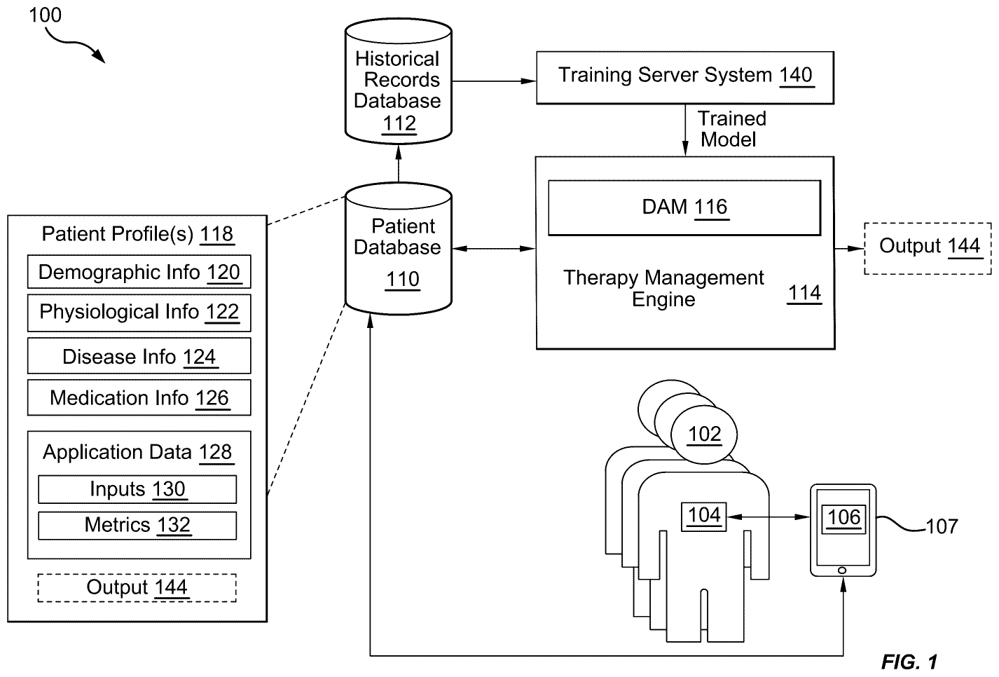

FIG. 1 illustrates an example therapy management system 100 for providing therapy management guidance to optimize GLP-1 medication effectiveness for a patient 102 (individually referred to herein as a patient and collectively referred to herein as patients), using a continuous analyte monitoring system 104 configured to continuously measure at least one of glucose, lactate, ketones, glycerol, amino acids, or free fatty acid levels. A patient, in certain embodiments, can be an obese patient, a patient on various GLP-1 regimens, a patient who has achieved various health goals (e.g., weight loss goals), and/or a patient who experienced various gastrointestinal symptoms and/or health complications, for example.

In certain embodiments, system 100 includes continuous analyte monitoring system 104, a display device 107 that executes application 106, a therapy management engine 114, a patient database 110, a historical records database 112, a training server system 140, and a therapy management engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods can include, but are not be limited to, potassium, glucose, endogenous insulin, acarboxyprothrombin; beta hydroxybutyrate; acetoacetate; acetone; acylcarnitine; exogenous insulin; adenine phosphoribosyl transferase; adenosine deaminase; albumin; albumin-creatinine ratio; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-peptide; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; creatinine; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglocbin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate (e.g., L-lactate and D-lactate); pyruvate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; proteinuria; quinine; reverse tri-iodothyronine (rT3); selenium; serum pancreatic lipase; sisomicin; somatomedin C; specific antibodies recognizing any one or more of the following that can include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, melatonin, Mycobacterium leprae, Mycoplasma pneumoniae, Myoglobin, Onchocerca volvulus, parainfluenza virus, Plasmodium falciparum, poliovirus, Pseudomonas aeruginosa, pro-C3, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones (e.g., insulin) naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include glucose, lactate, ketones, glycerol, amino acids, and free fatty acids (FFAs), in some cases other analytes listed above can also be considered.

In certain embodiments, continuous analyte monitoring system 104 is configured to continuously measure one or more analytes and transmit the analyte measurements to an electronic medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system can also be used to create reports for clinical care and/or therapy management guidance for a patient. In certain embodiments, the EMR can be in communication with therapy management engine 114 (e.g., via a network) for performing the techniques described herein. In particular, as described herein, therapy management engine 114 can obtain data associated with a patient, use the obtained data as input into one or more trained model(s), and output a prediction. In some cases, the EMR can provide the data to therapy management engine 114 to be used as input into the one or more models. Further, in some cases, therapy management engine 114, after making a prediction, can provide the output prediction to the EMR.

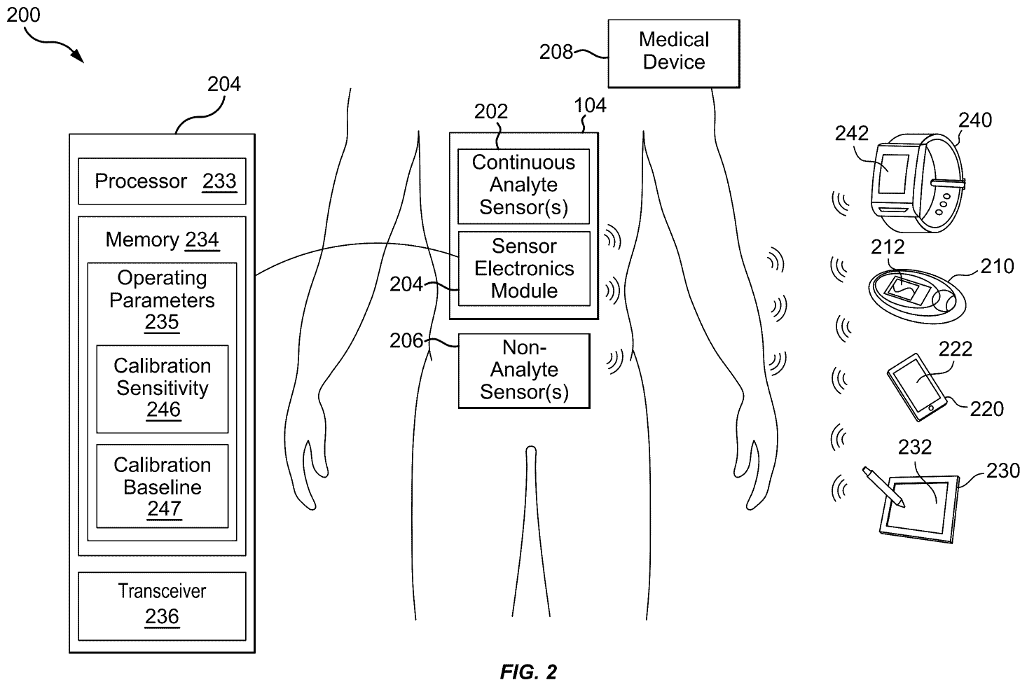

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection, WiFi connection, local area network connection, etc.). In certain embodiments, display device 107 is a smart phone. However, in certain other embodiments, display device 107 can instead be any other type of computing device such as a laptop computer, a smart watch, a tablet, a standalone receiver, or any other computing device capable of executing application 106. In some embodiments, continuous analyte monitoring system 104 and/or analyte sensor application 106 transmits the analyte measurements to one or more other individuals having an interest in the health of the patient (e.g., a family member or physician for real-time treatment and care of the patient). Continuous analyte monitoring system 104 is described in more detail with respect to FIG. 2.

Application 106 is a mobile health application that is configured to receive and analyze analyte measurements from continuous analyte monitoring system 104. In particular, application 106 stores information about a patient, including the patient's analyte measurements, in a patient profile 118 associated with the patient for processing and analysis, as well as for use by therapy management engine 114 to provide therapy management guidance to the patient.

Therapy management engine 114 refers to a set of software instructions with one or more software modules, including data analysis module (DAM) 116. In certain embodiments, therapy management engine 114 executes entirely on one or more computing devices in a private or a public cloud. In such embodiments, application 106 communicates with therapy management engine 114 over a network (e.g., Internet). In some other embodiments, therapy management engine 114 executes partially on one or more local devices, such as display device 107 and/or continuous analyte monitoring system 104, and partially on one or more computing devices in a private or a public cloud. In some other embodiments, therapy management engine 114 executes entirely on one or more local devices, such as display device 107 and/or continuous analyte monitoring system 104. As discussed in more detail herein, therapy management engine 114 provides therapy management guidance including diet recommendations, exercise recommendations, medication recommendations, and/or lifestyle changes based on information included in patient profile 118. For example, therapy management engine 114 provides therapy management guidance to the patient via application 106 relating to optimal GLP-1 dosing, optimal diet and/or meal timing, optimal exercise and/or exercise timing, medication timing, seeking medical intervention, etc. to optimize positive effects of the GLP-1 medication while detecting and minimizing negative side effects of the medication.

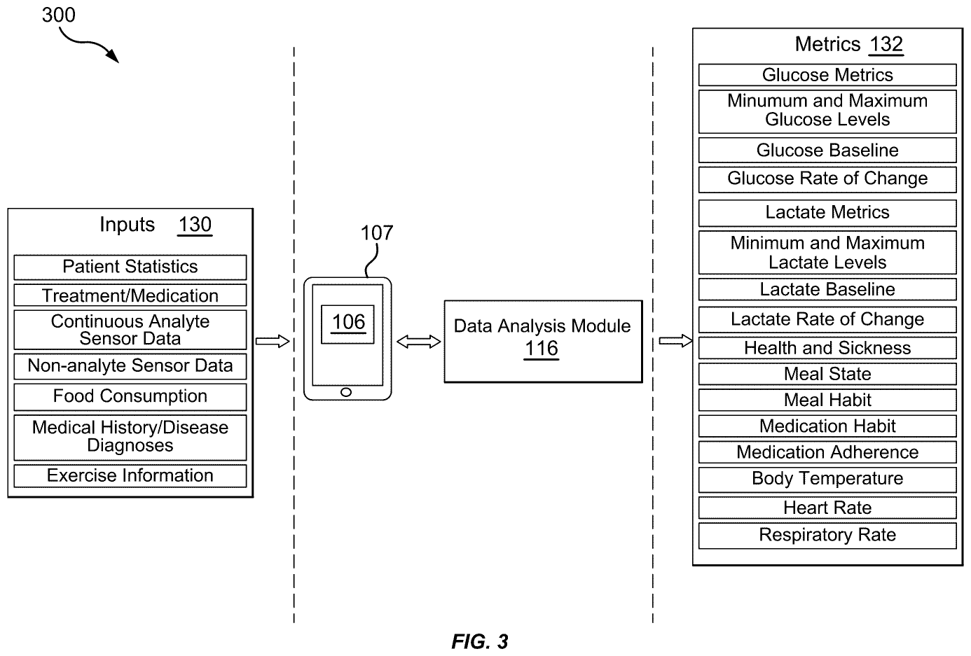

Patient profile 118 includes information collected about the patient from application 106. For example, application 106 provides a set of inputs 130, including the analyte measurements received from continuous analyte monitoring system 104, that are stored in patient profile 118. In certain embodiments, inputs 130 provided by application 106 include other data in addition to analyte measurements received from continuous analyte monitoring system 104. For example, application 106 can obtain additional inputs 130 through manual patient input, one or more other non-analyte sensors or devices, other applications executing on display device 107, etc. Non-analyte sensors and devices include one or more of, but are not limited to, an insulin pump sensor, stretch sensor, body sound sensor, acoustic gastography sensor, a heart rate monitor, a thermometer, a digital weight scale, sensors or devices provided by display device 107 (e.g., accelerometer, camera, global positioning system (GPS), heart rate monitor, etc.), or other patient accessories (e.g., a smart watch or fitness tracker), or any other sensors or devices that provide relevant information about the patient. Inputs 130 of patient profile 118 provided by application 106 are described in further detail below with respect to FIG. 3.

DAM 116 of therapy management engine 114 is configured to process the set of inputs 130 to determine one or more metrics 132. Metrics 132, discussed in more detail below with respect to FIG. 3, are, at least in some cases, generally indicative of the health of a patient, such as one or more of the patient's general analyte trends, trends associated with one or more gastrointestinal symptoms of the patient, etc. In certain embodiments, metrics 132 are then used by therapy management engine 114 as input for determining optimal GLP-1 regimen for a patient. As shown, metrics 132 are also stored in patient profile 118.

Patient profile 118 also includes demographic info 120, physiological info 122, disease progression info 124, and/or medication info 126. In certain embodiments, such information can be provided through patient input, obtained from one or more analyte or non-analyte sensors, or obtained from certain data stores (e.g., electronic medical records (EMRs), etc.). In certain embodiments, demographic info 120 includes one or more of the patient's age, ethnicity, gender, etc. In certain embodiments, physiological info 122 includes one or more of the patient's height, weight, and/or body mass index (BMI). In certain embodiments, disease progression info 124 includes information about a disease of a patient, such diagnoses of gastrointestinal diseases, thyroid cancer and/or thyroid disease, gall bladder disease and/or gall bladder dysfunction, liver disease and/or liver dysfunction, pancreatic cancer and/or pancreatitis, gastrointestinal symptoms (nausea, diarrhea, vomiting, etc.), gastroparesis, etc. In certain embodiments, information about a patient's disease also includes the length of time since diagnosis, the level of disease control, level of compliance with disease management therapy, other types of diagnosis (e.g., heart disease, hypertension, obesity), or measures of health (e.g., heart rate, exercise, sleep, etc.), and/or the like.

In certain embodiments, medication info 126 includes information about the amount, frequency, and type of a medication taken by a patient. In certain embodiments, the amount, frequency, and type of a medication taken by a patient is time-stamped and correlated with the patient's analyte levels, thereby, indicating the impact the amount, frequency, and type of the medication had on the patient's analyte levels.

In certain embodiments, medication information includes information about consumption of one or more drugs known to alter the patient's digestion and/or drugs that alter the patient's analyte levels. In certain embodiments, medication information includes information on a current GLP-1 regimen of the patient. In some embodiments, medication information is determined from a radiofrequency identification (RFID) chip present in a GLP-1 medication package. For example, the package that the GLP-1 medication is provided in can have an RFID chip that contains information about the medication type, concentration, desired dosing frequency and/or strategy, and/or dose volume. In certain embodiments, the RFID chip can be brought into proximity of continuous analyte monitoring system 104 having an NFC reader and the medication information can be transferred to the analyte sensor 202 and/or non-analyte sensor 206 and provided to the therapy management engine 114 (e.g., through display device 107). While the RFID chip could provide medication information to the therapy management engine 114, it can also provide information on the patient's compliance with the desired dosing frequency, concentration, etc. For example, every time the patient grabs the package to consume the GLP-1 medication, the RFID chip in the package can send a signal to the continuous analyte monitoring system 104. The signal, which can be indicative of the patient's compliance and consumption of the medication, can then be processed and/or transmitted by the continuous analyte monitoring system 104 to therapy management engine 114 (e.g., through display device 107).

In certain embodiments, patient profile 118 is dynamic because at least part of the information that is stored in patient profile 118 can be revised over time and/or new information can be added to patient profile 118 by therapy management engine 114 and/or application 106. Accordingly, information in patient profile 118 stored in patient database 110 provides an up-to-date repository of information related to a patient.

Patient database 110, in some embodiments, refers to a storage server that operates in a public or private cloud. Patient database 110 can be implemented as any type of datastore, such as relational databases, non-relational databases, key-value datastores, file systems including hierarchical file systems, and the like. In some exemplary implementations, patient database 110 is distributed. For example, patient database 110 can comprise a plurality of persistent storage devices, which are distributed. Furthermore, patient database 110 can be replicated so that the storage devices are geographically dispersed.

Patient database 110 includes patient profiles 118 associated with a plurality of patients who similarly interact with application 106 executing on the display devices 107 of the other patients. Patient profiles stored in patient database 110 are accessible to not only application 106, but therapy management engine 114, as well. Patient profiles in patient database 110 can be accessible to application 106 and therapy management engine 114 over one or more networks (not shown). As described above, therapy management engine 114, and more specifically DAM 116 of therapy management engine 114, can fetch inputs 130 from patient database 110 and compute a plurality of metrics 132 which can then be stored as application data 128 in patient profile 118.

In certain embodiments, patient profiles 118 stored in patient database 110 can also be stored in historical records database 112. Patient profiles 118 stored in historical records database 112 can provide a repository of up-to-date information and historical information for each patient of application 106. Thus, historical records database 112 essentially provides all data related to each patient of application 106, where data is stored according to an associated timestamp. The timestamp associated with information stored in historical records database 112 can identify, for example, when information related to a patient has been obtained and/or updated.

Further, historical records database 112 can maintain time series data collected for patients over a period of time, including for patients who use continuous analyte monitoring system 104 and application 106. For example, analyte data for a patient who has used continuous analyte monitoring system 104 and application 106 for a period of time to optimize a patient's GLP-1 regimen for various health outcomes (e.g., weight loss and/or prevention of symptoms) can have time series analyte data associated with the patient maintained over the period of time. In certain embodiments, the period of time can be 3 days, or 1 week, or one month, or one year, or five years, for example.

Further, in certain embodiments, historical records database 112 can include data for one or more patients who are not patients of continuous analyte monitoring system 104 and/or application 106. For example, historical records database 112 can include information (e.g., patient profile(s)) related to one or more patients prescribed various GLP-1 regimens, as well as information (e.g., patient profile(s)) related to one or more patients who have achieved various health goals (e.g., weight loss, fat reduction, and/or glucose control), and/or one or more patients who experienced various gastrointestinal symptoms and/or health complications. Data stored in historical records database 112 can be referred to herein as population data, which could include hundreds or thousands of data points for each one of thousands or millions of hosts in the host population. In other words, data stored in historical records database 112 and used in certain embodiments described herein could include gigabytes, terabytes, petabytes, exabytes, etc. of data.

Data related to each patient stored in historical records database 112 provides time series data collected over the disease lifetime of the patient (e.g., the patient's obesity). For example, the data can include information about the patient prior to beginning a GLP-1 therapy regimen, including information related to the patient's weight, body fat, and/or historical glucose control, as well as information related to other diseases, such as gastrointestinal diseases, thyroid cancer and/or thyroid disease, gall bladder disease and/or gall bladder dysfunction, liver disease and/or liver dysfunction, pancreatic cancer and/or pancreatitis, gastrointestinal symptoms (nausea, diarrhea, vomiting, etc.), gastroparesis, etc. Such information can indicate symptoms of the patient, physiological states of the patient, analyte levels of the patient, states/conditions of one or more organs of the patient, habits of the patient (e.g., activity levels, food consumption, etc.), medication prescribed, medication compliance, etc. over a period of time.

Although depicted as separate databases for conceptual clarity, in some embodiments, patient database 110 and historical records database 112 can operate as a single database. That is, historical and current data related to patients of continuous analyte monitoring system 104 and application 106, as well as historical data related to patients that were not previously patients of continuous analyte monitoring system 104 and application 106, can be stored in a single database. The single database can be a storage server that operates in a public or private cloud.

As mentioned previously, therapy management system 100 is configured to provide optimal GLP-1 regimen guidance to a patient using continuous analyte monitoring system 104. In certain embodiments, therapy management engine 114 is configured to provide real-time and or non-real-time GLP-1 regimen guidance to the patient and/or others, including but not limited, to healthcare providers, family members of the patient, caregivers of the patient, researchers, artificial intelligence (AI) engines, and/or other individuals, systems, and/or groups supporting care or learning from the data. In some embodiments, therapy management engine 114 is configured to provide therapy management guidance in the form of alerts, alarms, or notifications to the patient via display device 107. Alerts, alarms, and notifications can be provided in form of tactile, audible, or visual notifications, alarms, or alerts. Notifications, alarms and alerts can be provided to the patient on the display device 107, while an application is running, or as background notifications even when the application is running in the background. Alarms, alerts, and notifications inform the patient of various therapy management guidance provided by therapy management engine 114, including guidance to modify the patient's GLP-1 regimen, to consume a specific diet, to exercise or consume meals at a specific time, etc. For example, therapy management engine 114 can provide an alert, alarm, and/or notification to the patient to increase or decrease their GLP-1 regimen to optimize the positive effects of the GLP-1 medication and/or minimize negative side effects.

For example, therapy management engine 114 can be used to collect information associated with a patient in patient profile 118 stored in patient database 110, to perform analytics thereon for providing GLP-1 regimen optimization guidance to optimize the positive effects of the GLP-1 medication and/or minimize negative side effects, and in some cases, providing recommendations to the patient for medical intervention, medications, and/or lifestyle changes (e.g., diet, exercise and/or meal times). Patient profile 118 can be accessible to therapy management engine 114 over one or more networks (not shown) for performing such analytics.

In certain embodiments, therapy management engine 114 can utilize one or more trained machine learning models capable of providing GLP-1 regimen optimization based on information that therapy management engine 114 has obtained from patient profile 118. In the illustrated embodiment of FIG. 1, therapy management engine 114 can utilize trained machine learning model(s) provided by a training server system 140. Although depicted as a separate server for conceptual clarity, in certain embodiments, training server system 140 and therapy management engine 114 can operate as a single server or system. That is, the model can be trained and used by a single server and/or system, or can be trained by one or more servers and/or systems and deployed for use on one or more other servers and/or systems. In certain embodiments, the model can be trained on one or many virtual machines (VMs) running, at least partially, on one or many physical services in relational and or non-relational database formats.

Training server system 140 is configured to train the machine learning model(s) using training data, which can include data (e.g., from patient profiles) associated one or more patients (e.g., users or non-users of continuous analyte monitoring system 104 and/or application 106) on various GLP-1 regimens, who reach various health goals, as well as patients who experienced various gastrointestinal symptoms while on various GLP-1 regimens. The training data can be stored in historical records database 112 and can be accessible to training server system 140 over one or more networks (not shown) for training the machine learning model(s).

The training data refers to a dataset that has been featurized and labeled. For example, the dataset can include a plurality of data records, each including information corresponding to a different patient profile stored in patient database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a patient, which is reflected in a corresponding data record, can be a feature used in training the machine learning model. Such features include demographic information (e.g., age, gender, ethnicity, etc.), analyte information (e.g., glucose metrics, lactate metrics, ketones metrics, glycerol metrics, FFA metrics, etc.), non-analyte sensor information (e.g., body sound sensor data, fitness trackers, etc.), medical history and/or disease information (e.g., gastrointestinal diseases, thyroid cancer and/or thyroid disease, gall bladder disease and/or gall bladder dysfunction, liver disease and/or liver dysfunction, pancreatic cancer and/or pancreatitis, gastrointestinal symptoms (nausea, diarrhea, vomiting, etc.), gastroparesis, etc.), medication information, and/or any other information relevant to providing GLP-1 regimen optimization while avoiding gastrointestinal symptoms, or to providing recommendations to patients.

In addition, the data record is labeled with information the corresponding model is being trained to predict. In one example, if a model is being trained to predict an optimal GLP-1 regimen (e.g., for achieving a certain weight loss goal while avoiding gastrointestinal symptoms), the data records in the training dataset are labeled with GLP-1 regimen information. In another example, if a model is being trained to predict whether the patient's GLP-1 regimen is causing gastrointestinal symptoms, then the data records in the training dataset are labeled with gastrointestinal symptoms. In another example, if a model is being trained to output a prediction related to likelihood of achieving various weight loss goals at a current GLP-1 regimen, then the data records in the training dataset are labeled with one or more of various weight loss goals and information relating to achievement thereof. Note that, in one example, such models can be multi-input single-output (MISO) models, configured to make only one prediction (e.g., whether the patient's GLP-1 regimen is causing gastrointestinal symptoms, in which case additional MISO models can be trained to each predict the likelihood of achieving various health goals, risk of developing gastrointestinal symptoms, risk of developing other health complications related to GLP-1 regimen, or the like). In another example, such a model can be a multi-input multi-output (MIMO) model, configured to provide multiple predictions (e.g., presence or likelihood of developing gastrointestinal symptoms at a specified GLP-1 regimen, likelihood of achieving various health goals at a specified GLP-1 regimen, various health complication predictions, etc.).

The model(s) are then trained by training server and/or system 140 using the featurized and labeled training data. In particular, the features of each data record can be used as input into the machine learning model(s), and the generated output can be compared to label(s) associated with the corresponding data record. The model(s) can compute a loss based on the difference between the generated output and the provided label(s). This loss is then used to modify the internal parameters or weights of the model. By iteratively processing each data record corresponding to each historical patient, the model(s) can be iteratively refined to generate accurate predictions of an optimal GLP-1 regimen, a presence or likelihood of developing gastrointestinal symptoms at a specified GLP-1 regimen, likelihood of achieving various weight loss goals at a specified GLP-1 regimen, various health complication predictions, etc.

As illustrated in FIG. 1, training server system 140 deploys these trained model(s) to therapy management engine 114 for use during runtime. For example, therapy management engine 114 can obtain patient profile 118 associated with a patient and stored in patient database 110, use information in patient profile 118 as input into the trained model(s), and output a prediction indicative of the patient's optimal GLP-1 regimen, predicted change in health metrics (e.g., weight loss, metabolic score, liver health score, kidney health score), presence of likelihood of developing negative side effects (e.g., gastrointestinal symptoms, etc.), presence of health complications related to the GLP-1 regimen, and/or guidance related to minimizing negative side effects and/or achieving health goals (e.g., shown as output 144 in FIG. 1). Output 144 can be provided to the patient (e.g., through application 106), to a caretaker of the patient (e.g., a parent, a relative, a guardian, a teacher, a physical therapist, a fitness trainer, a nurse, etc.), to a physician or healthcare provider of the patient, or any other individual that has an interest in the wellbeing of the patient for purposes of improving the health of the patient, such as, in some cases by meeting positive health effect goals or minimizing negative side effects related to the GLP-1 regimen. Output 144 generated by therapy management engine 114 is stored in patient database 110 and is utilized to train or re-train the trained model(s) and/or a model-based system.

Accordingly, for example, optimal GLP-1 regimens, presence of gastrointestinal symptoms, and recommendations to optimize GLP-1 regimen and minimize symptoms, originally stored as outputs 144 in patient profile 118 in patient database 110 and then passed to historical records database 112, can provide an indication of the optimization of the GLP-1 regimen, progression or improvement of the patient's gastrointestinal symptoms over time, as well as provide an indication as to the effectiveness of the recommendations provided to the patient to optimize the GLP-1 regimen and minimize symptoms.

In certain embodiments, a patient's own historical data can be used by training server system 140 to train a personalized model for the patient that provides therapy management guidance and insight around the patient's current GLP-1 regimen and weight loss, current gastrointestinal symptoms, average analyte levels, etc. For example, in certain embodiments, a model trained based on population data can be used to provide GLP-1 regimen optimization guidance to the patient. However, after collecting personalized information (e.g., analyte sensor information, non-analyte sensor information, disease state, etc.) associated with the patient, the personalized information can be used for further personalizing the model. For example, information obtained over time from the patient is used to more accurately determine an optimal GLP-1 regimen based on the patient's weight loss goals and historical gastrointestinal symptoms, determine development of health complications related to GLP-1 regimen, and/or provide personalized recommendations for medical intervention, medications, and/or lifestyle changes.

Further, a patient's historical data can be used to generate a baseline to indicate progression or regression in the patient's rate of gastric emptying based, for example, on the patient's analyte metrics (e.g., baseline, rate of change, minimum and/or maximum levels), etc. As an illustrative example, a patient's analyte data over one week from two weeks ago can be used to generate a baseline that can be compared with the patient's current analyte data to identify whether the patient's weight loss response to the GLP-1 regimen and/or one or more gastrointestinal symptoms have improved. In certain embodiments, the model is further able to predict or project out the patient's weight loss and/or one or more gastrointestinal symptoms based on the patient's recent pattern of data (e.g., analyte data, non-analyte data, meal trends, exercise trends, etc.).

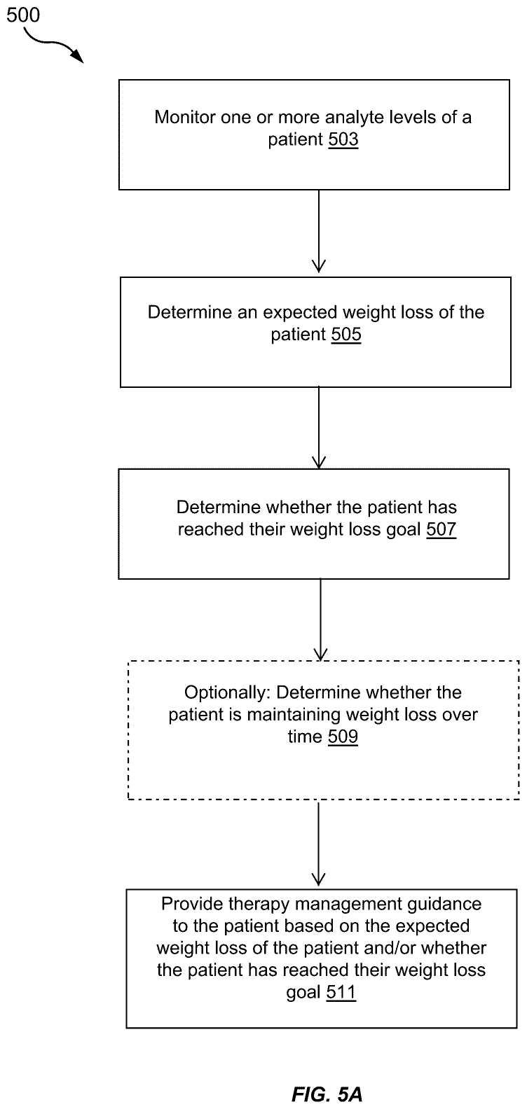

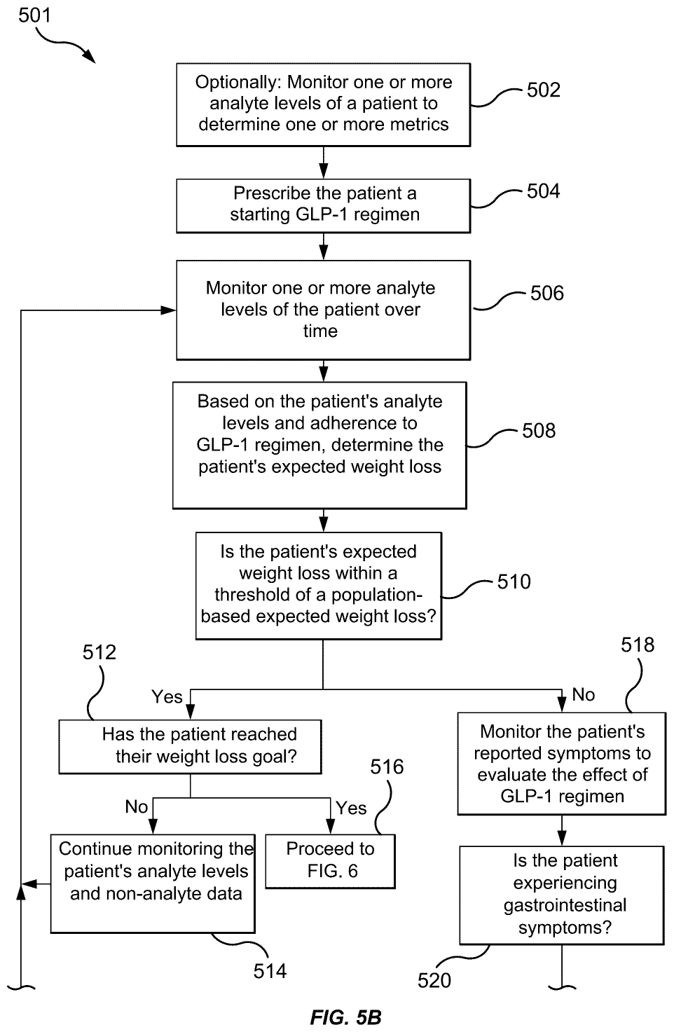

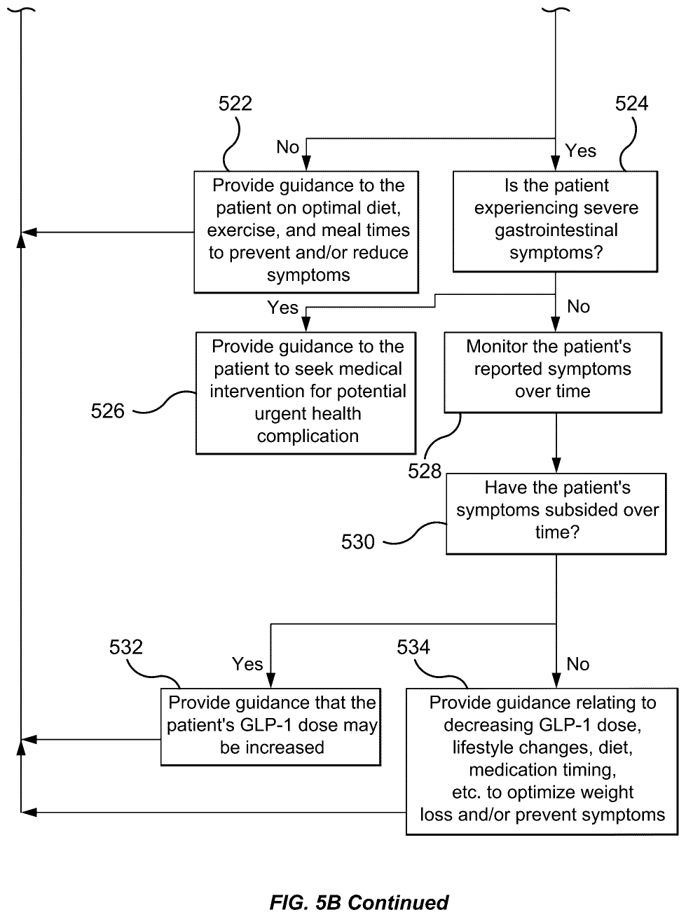

In certain embodiments, historical patient population data based on patients prescribed various GLP-1 regimens who have achieved various weight loss goals and/or experienced various gastrointestinal symptoms can be used to generate a baseline to indicate progression or regression in the patient's weight loss and/or gastrointestinal symptoms. For example, one or more of a patient's analyte metrics can be compared with historical patient population data of patients on a similar GLP-1 regimen who experienced certain gastrointestinal symptoms. If the patient's analyte metrics are consistent with historical patient population data of patients who experienced gastrointestinal symptoms, therapy management engine 114 makes certain therapy management guidance as described herein relative to FIGS. 4A-4B. Similarly, one or more of a patient's analyte metrics can be compared with historical patient population data of patients on the similar GLP-1 regimen who experienced a desired weight loss. If the patient's analyte metrics are consistent with historical patient population data of patients who experienced a desired weight loss, therapy management engine 114 can provide certain therapy management guidance to the patient as described herein relative to FIG. 5A-5B.

In certain other embodiments, known clinical evidence and/or observable data through clinical investigations of procedures can be used as a baseline to indicate progression or regression in the patient's weight loss and/or gastrointestinal symptoms.

In certain embodiments, an AI/ML model is trained to provide a recommendation for medical intervention, medication changes, medication timing, lifestyle changes, diet recommendations, and other types of therapy management guidance to help the patient achieve their weight loss goals and/or minimize or resolve one or more gastrointestinal symptoms based on the patient's historical data, including how different doses of GLP-1 medication, different types of food and/or activities impacted the patient's weight loss and/or gastrointestinal symptoms in the past. In certain embodiments, an AI/ML model is trained to predict the underlying cause of certain improvements or deteriorations in the patient's weight loss and/or gastrointestinal symptoms. For example, application 106 can display a patient interface with a graph that shows the patient's analyte levels with trend lines and indicate, e.g., retrospectively, how the body's analyte levels affected the patient's weight loss and/or gastrointestinal symptoms at certain points in time.

In certain other embodiments, rules-based models can be used. For example, a rules-based model can be used to map a patient's inputs, analyte data, non-analyte data, and/or historical data to certain weight loss goals and/or one or more gastrointestinal symptoms, recommendations for medical intervention, medication changes, lifestyle changes, etc., using, for example, a rules library. In certain embodiments, a rules-based model can map certain inputs to weight loss predictions, one or more gastrointestinal symptom predictions, and/or recommendations for patients with similar inputs in the past. Some example rules are discussed herein in relation to FIGS. 4A-6.

FIG. 2 is a diagram 200 conceptually illustrating an example continuous analyte monitoring system 104 including example continuous analyte sensor(s) with sensor electronics, in accordance with certain aspects of the present disclosure. For example, continuous analyte monitoring system 104 can be configured to continuously monitor one or more analytes of a patient, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 can be in wireless communication (e.g., directly or indirectly) with one or more of display devices 210, 220, 230, and 240. In certain embodiments, sensor electronics module 204 can also be in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 can comprise one or more sensors for detecting and/or measuring analyte(s). The continuous analyte sensor 202 can be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 202 can be configured to continuously measure analyte levels of a patient using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 202 provides a data stream indicative of the concentration of one or more analytes of the patient. The data stream can include raw data signals, which are then converted into a calibrated and/or filtered data stream used to provide estimated analyte value(s) to the patient.

In certain embodiments, the continuous analyte sensor 202 can be a multi-analyte sensor, configured to continuously measure multiple analytes in a patient's body. For example, in certain embodiments, the continuous multi-analyte sensor 202 can be a single sensor configured to measure lactate, glucose, fructose, FFAs, cholesterol, glycerol, and/or amino acids in the patient's body.

In certain embodiments, one or more single-analyte and/or multi-analyte sensors can be used in combination. Information from each of the multi-analyte sensor(s) and/or single analyte sensor(s) can be combined to provide therapy management guidance using methods described herein. In further embodiments, other non-contact and or periodic or semi-continuous, but temporally limited, measurements for physiological information can be integrated into the system such as by including weight scale information or non-contact heart rate monitoring from a sensor pad under the patient while in a chair or bed, through an infra-red camera detecting temperature and/or blood flow patterns of the patient, and/or through a visual camera with machine vision for height, weight, or other parameter estimation without physical contact.

In certain embodiments, the continuous analyte sensor(s) 202 comprises a percutaneous wire that has a proximal portion coupled to the sensor electronics module 204 and a distal portion with several electrodes, such as a measurement electrode and a reference electrode. The measurement (or working) electrode can be coated, covered, treated, embedded, etc., with one or more chemical molecules that react with a particular analyte, and the reference electrode can provide a reference electrical voltage. The measurement electrode can generate the analog electrical signal, which is conveyed along a conductor that extends from the measurement electrode to the proximal portion of the percutaneous wire that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis.

In certain embodiments, the continuous analyte sensor(s) 202 comprises a planar substrate that has a proximal portion coupled to the sensor electronics module 204 and a distal portion with several electrodes such as a measurement electrode and a reference electrode. The measurement (or working) electrode can be coated, covered, treated, embedded, etc., with one or more chemical molecules that react with a particular analyte, and the reference electrode can provide a reference electrical voltage. The measurement electrode can generate the analog electrical signal, which is conveyed along a conductor that extends from the measurement electrode to the proximal portion of the continuous analyte sensor(s) 202 that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis.

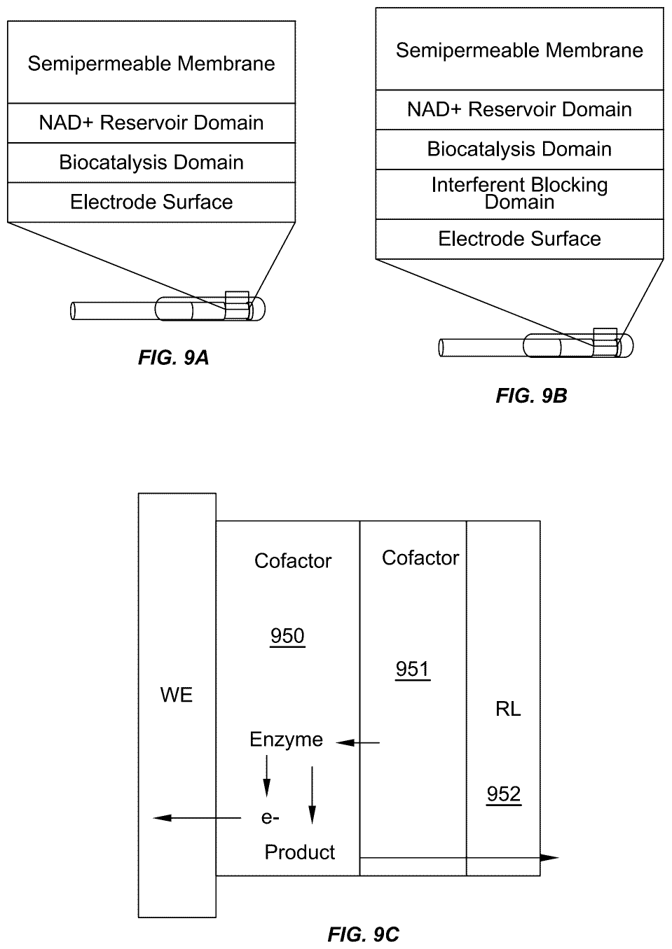

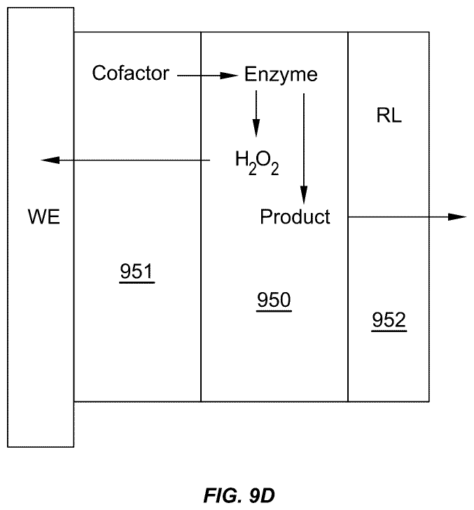

In certain embodiments, in addition to the planar and coaxial sensors described herein, other configurations of the planar and coaxial continuous analyte sensor(s) 202 can also be used, such as a multi-analyte sensor that includes multiple measurement electrodes, each generating an analog electrical signal that represents the concentration levels of a particular analyte. Exemplary planar and coaxial sensors are further described in reference to FIGS. 9A-13E.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 202 can include a single-analyte sensor configured to measure lactate concentration levels, and another single-analyte sensor configured to measure glucose concentration levels of the patient. As another illustrative example, continuous analyte sensor(s) 202 can include a single-analyte sensor configured to measure lactate concentration levels, and one or more multi-analyte sensors configured to measure glucose concentration levels, ketone concentration levels, creatinine concentration levels, etc. As yet another illustrative example, continuous analyte sensor(s) 202 can include a multi-analyte sensor configured to measure lactate concentration levels, glucose concentration levels, fructose concentration levels, FFA concentration levels, etc. Accordingly, continuous analyte sensor(s) 202 is configured to generate at least one analog electrical signal that is proportional to the concentration level of a particular analyte, and sensor electronics module 204 is configured to convert the analog electrical signal into analyte sensor count values, calibrate the analyte sensor count values based on the sensitivity profile of the continuous analyte sensor(s) 202 to generate measured analyte concentration levels, and transmit measured analyte concentration level data, including the measured analyte concentration levels, to a display device, such as display devices 210, 220, and/or 230, via a wireless connection. For example, sensor electronics module 204 can be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which can be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 202 can incorporate a thermocouple within, or alongside, the percutaneous wire to provide an analog temperature signal to the sensor electronics module 204, which can be used to correct the analog electrical signal or the measured analyte data for temperature. In other embodiments, the thermocouple can be incorporated into the sensor electronics module 204 above the adhesive pad, or, alternatively, the thermocouple can contact the epidermis of the patient through openings in the adhesive pad.

In certain embodiments, the sensor electronics module 204 includes, inter alia, processor 233, storage element or memory 234, wireless transmitter/receiver (transceiver) 236, one or more antennas coupled to wireless transceiver 236, analog electrical signal processing circuitry, analog to-digital (A/D) signal processing circuitry, digital signal processing circuitry, a power source for continuous analyte sensor(s) 202 (such as a potentiostat), etc.

Processor 233 can be a general-purpose or application-specific microprocessor, an application-specific integrated circuit (ASIC), a field programmable gate array (FPGA), etc., that executes instructions to perform control, computation, input/output, etc. functions for the sensor electronics module 204. Processor 233 can include a single integrated circuit, such as a micro processing device, or multiple integrated circuit devices and/or circuit boards working in cooperation to accomplish the appropriate functionality. In certain embodiments, processor 233, memory 234, wireless transceiver 236, the A/D signal processing circuitry, and the digital signal processing circuitry can be combined into a system-on-chip (SoC).

Generally, processor 233 can be configured to sample the analog electrical signal using the A/D signal processing circuitry at regular intervals (such as the sampling period) to generate analyte sensor count values based on the analog electrical signals produced by the continuous analyte sensor(s) 202, calibrate the analyte sensor count values based on the sensitivity profile of the continuous analyte sensor(s) 202 to generate measured analyte concentration levels, generate measured analyte data from the measured analyte concentration levels, and generate sensor data packages that include, inter alia, the measured analyte concentration level data. Processor 233 can store the measured analyte concentration level data in memory 234, and generate the sensor data packages at regular intervals (such as the transmission period) for transmission by wireless transceiver 236 to a display device, such as display devices 210, 220, 230, and/or 240. Processor 233 can also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

In various embodiments, memory 234 can include volatile and nonvolatile medium. For example, memory 234 can include combinations of random access memory (RAM), dynamic RAM (DRAM), static RAM (SRAM), read only memory (ROM), flash memory, cache memory, and/or any other type of non-transitory computer-readable medium. Memory 234 can store one or more analyte sensor system applications, modules, instruction sets, etc. for execution by processor 233, such as instructions to generate measured analyte data from the analyte sensor count values, etc.

Memory 234 can also store certain sensor operating parameters 235, such as a calibration slope (or calibration sensitivity), a calibration baseline, etc. In particular, the calibration sensitivity, calibration baseline, and other information related to the sensitivity profile for the sensor electronics module 204 can be programmed into the sensor electronics module 204 during the manufacturing process, and then used to convert the analyte sensor electrical signals into measured analyte concentration levels. For example, as discussed above, the calibration slope can be used to predict an initial in vivo sensitivity (M₀) and a final in vivo sensitivity (M_{f}), which are stored in memory 234 and used to convert the analyte sensor electrical signals into measured analyte concentration levels. In certain embodiments, calibration sensitivity (M_{CC}) 246 and/or calibration baseline 247 can be stored in memory 234.

In certain embodiments, sensor electronics module 204 includes electronic circuitry associated with measuring and processing the continuous analyte sensor data, including prospective algorithms associated with processing and calibration of the sensor data. Sensor electronics module 204 can be physically connected to continuous analyte sensor(s) 202 and can be integral with (non-releasably attached to) to continuous analyte sensor(s) 202. Alternatively, sensor electronics module 204 can be physically connected to continuous analyte sensor(s) 202 and releasably attachable to continuous analyte sensor(s) 202. Sensor electronics module 204 can include hardware, firmware, and/or software that enable measurement of levels of analyte(s) via continuous analyte sensor(s) 202. For example, sensor electronics module 204 can include a potentiostat, an amperostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which can be transmitted by sensor electronics module 204. Each of display devices 210, 220, 230, or 240 can include a display such as a touchscreen display 212, 222, 232, and/or 242 for displaying sensor data to a patient and/or for receiving inputs from the patient. For example, a graphical user interface (GUI) can be presented to the patient for such purposes. In certain embodiments, the display devices can include other types of user interfaces such as a voice user interface instead of, or in addition to, a touchscreen display for communicating sensor data to the patient of the display device and/or for receiving patient inputs. Display devices 210, 220, 230, and 240 can be examples of display device 107 illustrated in FIG. 1 used to display sensor data to a patient of the system of FIG. 1 and/or to receive input from the patient.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices can include a custom display device specially designed for displaying certain types of displayable sensor data associated with analyte data received from sensor electronics module. In certain embodiments, the plurality of display devices can be configured for providing alerts/alarms based on the displayable sensor data. Display device 210 is an example of such a custom device. In certain embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 can be in communication with a medical device 208. Medical device 208 can be a medicament dispensing device for administering GLP-1 medication. In one example, the dispensing device is a pump or pen used to dispense the medication. Based on the analyte and/or non-analyte sensor data received from continuous analyte monitoring system 104, therapy management system 100 can communicate directly (e.g., through electronic or wireless communication) with the medicament dispensing device to dose the recommended amount of GLP-1 medication as described in FIGS. 4A-6. For example, the medicament dispensing device can maintain, up titrate, or down titrate the patient's GLP-1 medication dose automatically in response to dosage information received from therapy management engine 114. In addition to controlling GLP-1 medication dose, the medicament dispensing device can also provide the GLP-1 medication dose at an ideal time based on guidance from therapy management engine 114 as described herein. The ideal time can be based on the patient's analyte and/or non-analyte sensor data received from continuous analyte monitoring system 104 and/or historical patient population data based on patients on similar GLP-1 regimens who achieved a desired weight loss and/or reduced gastrointestinal symptoms.

In certain embodiments, the medical device 208 in communication with the sensor electronics module 204 can be a passive medical device. For example, medical device 208 can be an insulin pump for administering insulin to a patient. It can be desirable for such an insulin pump to receive and track glucose levels transmitted from continuous analyte monitoring system 104, where continuous analyte sensor 202 is configured to measure at least glucose.

Further, as mentioned, sensor electronics module 204 can also be in communication with other non-analyte sensors 206. Non-analyte sensors 206 can include, but are not limited to, a body sound sensor, an acoustic gastrography sensor, an insulin pump sensor, an accelerometer sensor, a global positioning system (GPS) sensor, a temperature sensor, a respiration rate sensor, etc. Non-analyte sensors 206 can also include monitors such as heart rate monitors, blood pressure monitors, pulse oximeters, caloric intake monitors, indirect calorimetry devices, electrocardiogram (EKG) and muscle contraction devices, and medicament delivery devices. One or more of these non-analyte sensors 206 can provide data to therapy management engine 114 described further below. In some aspects, a patient can manually provide some of the data for processing by training server system 140 and/or therapy management engine 114 of FIG. 1. Note that although shown separately, any one or more of the non-analyte sensors 206 can be incorporated into the continuous analyte sensors 202. For example, the EKG and muscle contraction devices can be incorporated into the continuous analyte sensors 202.

In certain embodiments, non-analyte sensors 206 can further include sensors for analyzing breath (e.g., breath analyzers), measuring skin temperature, measuring core temperature, measuring sweat rate, and/or measuring sweat composition.

In certain embodiments, the non-analyte sensors 206 can be combined in any other configuration, such as, for example, combined with one or more continuous analyte sensors 202. As an illustrative example, a non-analyte sensor, e.g., a body sound sensor, can be combined with a continuous glucose and/or lactate sensor 202 to form a glucose/lactate/body sound sensor used to transmit sensor data to the sensor electronics module 204 using common communication circuitry.

In certain embodiments, a wireless access point (WAP) can be used to couple one or more of continuous analyte monitoring system 104, the plurality of display devices, medical device(s) 208, and/or non-analyte sensor(s) 206 to one another. For example, the WAP can provide Wi-Fi, Bluetooth and/or cellular connectivity among these devices. Near Field Communication (NFC) and or Bluetooth can also be used among devices depicted in diagram 200 of FIG. 2.

FIG. 3 illustrates example inputs and example metrics that are calculated based on the inputs for use by the therapy management system of FIG. 1, according to some embodiments disclosed herein. In particular, FIG. 3 provides a more detailed illustration of example inputs and example metrics introduced in FIG. 1.

FIG. 3 illustrates example inputs 130 on the left, application 106 and DAM 116 in the middle, and metrics 132 on the right. In certain embodiments, each one of metrics 132 can correspond to one or more values, e.g., discrete numerical values, ranges, or qualitative values (high/medium/low, stable/unstable, etc.). Application 106 obtains inputs 130 through one or more channels (e.g., manual patient input, sensors, other applications executing on display device 107, an EMR system, etc.). As mentioned previously, in certain embodiments, inputs 130 can be processed by DAM 116 to output a plurality of metrics, such as metrics 132. Inputs 130 and metrics 132 can be used by training server system 140 and therapy management engine 114 to both train and deploy one or more machine learning models for providing GLP-1 optimization guidance, and other functionalities described herein.

Starting with inputs 130, patient statistics, such as one or more of age, gender, height, weight, BMI, body composition (e.g., % body fat), stature, build, or other information can also be provided as an input. In certain embodiments, patient statistics are provided through a user interface, by interfacing with an electronic source such as an electronic medical record, and/or from measurement devices. In certain embodiments, the measurement devices include one or more of a wireless, e.g., Bluetooth-enabled, weight scale and/or camera, which can, for example, communicate with the display device 107 to provide patient data.

In certain embodiments, treatment/medication information is also provided as an input. Medication information can include information about the type, dosage, and/or timing of when one or more medications are to be taken by the patient. Treatment information can include information regarding different lifestyle habits recommended by the patient's physician. For example, the patient's physician can recommend a patient follow specific diet recommendations (e.g., types of calories consumed), exercise at a specific time during the day for a specific duration, eat a meal at certain days and/or times, or cut calories by 500 to 1,000 calories daily to improve weight loss and/or analyte levels (e.g., lactate and/or glucose, for example) to improve GLP-1 effectiveness. In certain embodiments, treatment/medication information can be provided through manual patient input.

In certain embodiments, analyte sensor data is also provided as input, for example, through continuous analyte monitoring system 104. In certain embodiments, analyte sensor data can include glucose, lactate, fructose, FFA, cholesterol, glycerol, and/or amino acid levels measured by at least a single analyte sensor (or multi-analyte sensor) in continuous analyte monitoring system 104.

In certain embodiments, input can also be received from one or more non-analyte sensors, such as non-analyte sensors 206 described with respect to FIG. 2. Input from such non-analyte sensors 206 can include information related to a heart rate, a respiration rate, blood pressure, or a body temperature (e.g. to detect illness, physical activity, etc.) of a patient and/or measurements of variations, averages, derivatives, or any other multi-measurement analytical calculations between at least two points of non-analyte and/or analyte data. In certain embodiments, electromagnetic sensors can also detect low-power radio frequency (RF) fields emitted from objects or tools touching or near the object, which can provide information about patient activity or location.

In certain embodiments, food consumption information is also provided as input. Food consumption information can include information about one or more of meals, snacks, and/or beverages, such as one or more of the size, content (carbohydrate, fat, protein, etc.), sequence of consumption, and time of consumption. In certain embodiments, food consumption can be provided by a patient through manual entry, by providing a photograph through an application that is configured to recognize food types and quantities, and/or by scanning a bar code or menu. In various examples, meal size can be manually entered as one or more of calories, quantity (“three cookies”), menu items (“Royale with Cheese”), and/or food exchanges (1 fruit, 1 dairy). In some examples, meal information can be received via a convenient user interface provided by application 106.

In certain embodiments, food consumption information (the type of food (e.g., liquid or solid, snack or meal, etc.) and/or the composition of the food (e.g., carbohydrate, fat, protein, etc.)) can be determined automatically based on information provided by one or more sensors. Some example sensors can include body sound sensors (e.g., abdominal sounds can be used to detect the types of meal, e.g., liquid/solid food, snack/meal, etc.), radio-frequency sensors, cameras, hyperspectral cameras, and/or analyte (e.g., glucose, lactate, etc.) sensors to determine the type and/or composition of the food.

In certain embodiments, medical history and/or disease diagnoses (e.g., obesity, gastrointestinal diseases, thyroid cancer and/or thyroid disease, gall bladder disease and/or gall bladder dysfunction, liver disease and/or liver dysfunction, pancreatic cancer and/or pancreatitis, gastrointestinal symptoms (nausea, diarrhea, vomiting, etc.), gastroparesis, etc.) can be provided as an input. For example, the patient can have an existing diagnosis of obesity and/or one or more health complications and this diagnosis can be provided through manual patient input. In certain embodiments, disease diagnoses can also be provided by interfacing with an electronic source such as an electronic medical record. In certain embodiments, medical history and/or disease diagnoses information can also indicate the duration of time the patient has been experiencing the disease, the diagnosis issue, the frequency of symptom occurrence, and/or how well controlled the corresponding symptom has been. The medical history and/or disease diagnoses information can further be used to help classify the patient using population-based models. For example, there can be multiple different sub-population models that the patient's individual data could be compared to and classified accordingly. As an example, a patient with a history of obesity without diabetes would likely have a different response to medication as compared to a patient with obesity and diabetes for many years (e.g., autonomic nervous system dysfunction). Further, the heart rate variability metric can be utilized to understand autonomic nervous system dysfunction and can be a good surrogate metric for risk of gastroparesis. Specifically, reduced heart rate variability can indicate improved autonomic nervous system function and increased gastroparesis.

In certain embodiments, exercise information is also provided as an input. Exercise information can be any information surrounding activities requiring physical exertion by the patient. For example, exercise information can range from information related to low intensity (e.g., walking a few steps) and high intensity (e.g., five mile run) physical exertion. In certain embodiments, exercise information can also be provided through manual patient input suggesting the patient will begin a specific exercise type and/or with certain exercise parameters. In certain embodiments, exercise information can be provided or determined based on information provided, for example, by non-analyte sensors 206 (e.g., a temperature sensor, a heart rate monitor, a wearable blood pressure monitor, an accelerometer sensor on a wearable device such as a watch, fitness tracker, and/or patch, etc.). In certain embodiments, exercise information can be provided or determined based on information provided, for example, by continuous analyte monitoring system 104 (e.g., it can be deduced that the patient engaged in exercise based on their lactate and/or glucose data).

In certain embodiments, time can also be provided as an input, such as time of day or time from a real-time clock. For example, in certain embodiments, input analyte data can be timestamped to indicate a date and time when the analyte measurement was taken for the patient.

Patient input of any of the above-mentioned inputs 130 can be provided through continuous analyte monitoring system 104, non-analyte sensors 206, and/or a user interface, such a user interface of display device 107 of FIG. 1. As described above, in certain embodiments, DAM 116 determines or computes the patient's metrics 132 based on inputs 130. An example list of metrics 132 is shown in FIG. 3.

In certain embodiments, glucose metrics can be determined from sensor data (e.g., glucose measurements obtained from a continuous glucose sensor of continuous analyte monitoring system 104). For example, glucose metrics refer to time-stamped glucose measurements or values that are continuously generated and stored over time. In some examples, glucose metrics can also be determined, for example, based upon historical data in particular situations, e.g., given a combination of food consumption, insulin, and/or exercise.

In certain embodiments, a minimum and maximum glucose level can be determined from sensor data. For example, daily minimum and maximum glucose values for each day over a specified amount of time (e.g., a week or a month) can be determined. In certain embodiments, the minimum and maximum glucose levels can be determined based on an average minimum and maximum over a specified amount of time (e.g., a week or a month). In certain embodiments, DAM 116 can continuously or periodically calculate a normal glucose range and time-stamp and store the corresponding information in the patient's profile 118.

In other embodiments, a normal minimum and maximum glucose level can be determined from population data (e.g., from data records or historical patients taking a specific dose of GLP-1, historical patients who achieved various weight loss goals, and/or historical patients who experienced various gastrointestinal symptoms). In such embodiments, each patient can have personalized, customized, acceptable glucose minimum and/or maximum glucose values, which can be determined based on time periods when the patient is in a fasting state, during a meal, and/or following a meal, for example.

In certain embodiments, a glucose baseline can be determined from sensor data (e.g., glucose measurements obtained from a continuous glucose sensor of continuous analyte monitoring system 104). A glucose baseline represents a patient's normal glucose levels during periods where fluctuations in glucose production is typically not expected. A patient's baseline glucose level is generally expected to remain constant over time, unless challenged through an action such as consuming food or exercise by the patient, for example. Further, each patient can have a different glucose baseline. In certain embodiments, a patient's glucose baseline can be determined by calculating an average of glucose levels over a specified amount of time where fluctuations are not expected. In certain embodiments, a patient's glucose baseline can be representative of the patient's glucose levels in a fasting state. In certain embodiments, a patient's glucose baseline can be based on glucose metrics such as time-in-range, a maximum glucose level, a minimum glucose level, glucose rates of change, or other glucose trends in response to similar meals and/or situations by the patient.

For example, the baseline glucose level for a patient can be determined over a period of time when the patient is sleeping, sitting in a chair, or other periods of time where the patient is sedentary and not consuming food or medication which would reduce or increase glucose levels. In certain embodiments, DAM 116 can continuously, semi-continuously, or periodically calculate a glucose baseline and time-stamp and store the corresponding information in the patient's profile 118. In certain embodiments, DAM 116 can calculate the glucose baseline using glucose levels measured over a period of time where the patient is sedentary, the patient is not consuming glucose-heavy foods, and where no external conditions exist that would affect the glucose baseline. In certain other embodiments, DAM 116 can use glucose levels measured over a period of time where the patient is, at least for a subset of the period of time, engaging in exercise, and/or consuming glucose and/or an external condition exists that would affect the glucose baseline level. In this case, in some examples, DAM 116 can first identify which measured glucose values are to be used for calculating the baseline glucose level by identifying glucose values that can have been affected by an external event, such the consumption of food, exercise, medication, or other perturbation that would disrupt the capture of a glucose baseline measurement. DAM 116 can then exclude such measurements when calculating the glucose baseline level of the patient. In some other examples, DAM 116 can calculate the glucose baseline level by first determining a percentage of the number of glucose values measured during a specific time period that represent the lowest glucose values measured. DAM 116 can then take an average of this percentage to determine the glucose baseline level.

In certain embodiments, a glucose rate of change can be determined from glucose levels (e.g., glucose measurements obtained from a continuous glucose sensor of continuous analyte monitoring system 104). A glucose rate of change refers to a rate that indicates how one or more time-stamped glucose measurements or values change in relation to one or more other time-stamped glucose measurements or values. Glucose rates of change can be determined over one or more seconds, minutes, hours, days, etc. Further, glucose rate of change can be positive, negative, or an absolute value. In certain embodiments, a glucose rate of change above or below a threshold can be determined. A rapid rise in glucose outside of a threshold, not related to exercise or a meal, can be indicative of organ dysfunction (e.g., liver dysfunction).

In certain embodiments, lactate metrics can be determined from sensor data (e.g., lactate measurements obtained from a continuous lactate sensor of continuous analyte monitoring system 104). For example, lactate metrics refer to time-stamped lactate measurements or values that are continuously generated and stored over time. In some examples, lactate metrics can also be determined, for example, based upon historical data in particular situations, e.g., given a combination of food consumption and/or exercise. Further, lactate metrics determined from sensor data can indicate the relationship between exercise intensity and lactate rise. A robust rise could signal conditions like mitochondrial dysfunction.

In certain embodiments, a minimum and maximum lactate level can be determined from sensor data. For example, a daily minimum and maximum lactate values for each day over a specified amount of time (e.g., a week or a month) can be determined. In certain embodiments, the minimum and maximum lactate levels can be determined based on an average minimum and maximum over a specified amount of time (e.g., a week or a month). In certain embodiments, DAM 116 can continuously or periodically calculate a normal lactate range and time-stamp and store the corresponding information in the patient's profile 118.

In other embodiments, a normal minimum and maximum lactate level can be determined from population data (e.g., from data records or historical patients taking a specific dose of GLP-1, historical patients who achieved various weight loss goals, and/or historical patients who experienced various gastrointestinal symptoms). In such embodiments, each patient can have personalized, customized, acceptable minimum and/or maximum lactate values, which can be determined based on various time periods when the patient is in a fasting state, during a meal, and/or following a meal, for example.

In certain embodiments, a lactate baseline can be determined from sensor data (e.g., lactate measurements obtained from a continuous lactate sensor of continuous analyte monitoring system 104). A lactate baseline represents a patient's normal lactate levels during periods where fluctuations in lactate production is typically not expected. A patient's baseline lactate level is generally expected to remain constant over time, unless challenged through an action such as consuming food that is high in lactate, for example. Additionally, a patient's baseline lactate level can also change based on the patient's health. Further, each patient can have a different lactate baseline. In certain embodiments, a patient's lactate baseline can be determined by calculating an average of lactate levels over a specified amount of time where fluctuations are not expected.

For example, the baseline lactate level for a patient can be determined over a period of time when the patient is sleeping, sitting in a chair, or other periods of time where the patient is sedentary and not consuming food or medication, or exercising, which would reduce or increase lactate levels. In certain embodiments, DAM 116 can continuously, semi-continuously, or periodically calculate a lactate baseline and time-stamp and store the corresponding information in the patient's profile 118. In certain embodiments, DAM 116 can calculate the lactate baseline using lactate levels measured over a period of time where the patient is sedentary (e.g., not exercising) and where no external conditions exist that would affect the lactate baseline. In certain embodiments, DAM 116 can calculate the lactate baseline level by first determining a percentage of the number of lactate values measured during a specific time period that represent the lowest lactate values measured. DAM 116 can then take an average of this percentage to determine the lactate baseline level.

In certain embodiments, a lactate rate of change can be determined from lactate levels (e.g., lactate measurements obtained from a continuous lactate sensor of continuous analyte monitoring system 104). A lactate rate of change refers to a rate that indicates how one or more time-stamped lactate measurements or values change in relation to one or more other time-stamped lactate measurements or values. Lactate rates of change can be determined over one or more seconds, minutes, hours, days, etc. Further, lactate rate of change can be positive, negative, or an absolute value. In certain embodiments, a lactate rate of change above or below a threshold can be determined. A rapid rise in lactate over a threshold, not related to exercise or a meal, can be indicative of a health complication, such as infection.

In certain embodiments, a standard deviation of analyte levels (not shown) can be determined from the analyte data. In some examples, a standard deviation of one or more analyte levels can be determined based on the variability of one or more analyte levels as compared to an average analyte level over one or more time periods. In certain embodiments, a time-in-range metric (not shown) can be determined from the analyte data. For example, with an established upper limit and lower limit, the time period during which the analyte data is between the upper and lower limits can be determined. The time-in-range can be determined for individual instances of the analyte data being in range, or can be determined over a predetermined length of time (e.g., one day) for which each of the individual in range periods are summed.

In certain embodiments, analyte trends can be determined based on analyte levels over certain periods of time. In certain embodiments, analyte trends (e.g., glucose, potassium, calcium, ammonia, or lactate trends) can be determined based on analyte baselines over certain periods of time. In certain embodiments, analyte trends can be determined based on absolute analyte level minimums over certain periods of time. In certain embodiments, analyte trends can be determined based on absolute maximum analyte levels over certain periods of time. In certain embodiments, analyte trends can be determined based on analyte level rates of change over certain periods of time. In certain embodiments, analyte trends can be determined in relation to physical activity, diet, illness, sleep deprivation, etc. In certain embodiments, analyte trends can be determined based on analyte baseline rates of change over certain periods of time.

In certain embodiments, health and sickness metrics can be determined, for example, based on one or more of patient input (e.g., pregnancy information or known sickness and/or infection information), from physiologic sensors (e.g., temperature), activity sensors, or a combination thereof. In certain embodiments, based on the values of the health and sickness metrics, for example, a patient's state can be defined as being one or more of healthy, ill, rested, or exhausted.

In certain embodiments, the meal state metric can indicate the state the patient is in with respect to food consumption. For example, the meal state can indicate whether the patient is in one of a fasting state, pre-meal state, eating state, post-meal response state, or stable state. In certain embodiments, the meal state can also indicate nourishment on board, e.g., meals, snacks, or beverages consumed, and can be determined, for example from food consumption information, time of meal information, and/or digestive rate information, which can be correlated to food type, quantity, and/or sequence (e.g., which food/beverage was eaten first).

In certain embodiments, meal habits metrics are based on the content and the timing of a patient's meals. For example, if a meal habit metric is on a scale of 0 to 1, the better/healthier the meal consumed by the patient, the higher the meal habit metric of the patient will be to 1, in an example. Better/healthier meals can be defined as those that do not drive analyte (e.g., glucose) levels of a patient out of a normal range for the patient (e.g., 70-180 mg/dL glucose or the patient's desired range). Also, the more the patient's food consumption adheres to a certain time schedule, the closer their meal habit metric will be to 1, in the example. In certain embodiments, the meal habit metrics can reflect the contents of a patient's meals where, e.g., three numbers can indicate the percentages of carbohydrates, proteins and fats. The content of the patient's meals can be utilized to determine the patient's rate of gastric emptying in response to various types or content of meals.

In certain embodiments, medication habit metrics are based on the patient's prescribed medications and a determination of whether the prescribed medications can have an effect on the patient's analyte levels. For example, by analyzing a patient's medication habits, DAM 116 can determine whether the patient's medications can impact the patient's analyte measurements at a particular time. Based on the patient's medication habits, DAM 116 can determine whether the patient's analyte levels are a result of medication consumption or suboptimal GLP-1 regimen, for example. Medication habit metrics can be time-stamped so that they can be correlated with the patient's analyte levels at the same time.

In certain embodiments, medication adherence is measured by one or more metrics that are indicative of how committed the patient is towards their medication regimen. In certain embodiments, medication adherence metrics are calculated based on one or more of the timing of when the patient takes medication (e.g., whether the patient is on time or on schedule), the type of medication (e.g., is the patient taking the right type of medication), and the dosage of the medication (e.g., is the patient taking the right dosage).

In certain embodiments, body temperature metrics can be calculated by DAM 116 based on inputs 130, and more specifically, non-analyte sensor data from a temperature sensor. In certain embodiments, heart rate metrics can be calculated by DAM 116 based on inputs 130, and more specifically, non-analyte sensor data from a heart rate sensor. In certain embodiments, respiratory rate metrics can be calculated by DAM 116 based on inputs 130, and more specifically, non-analyte sensor data from a respiratory rate sensor.

### Example Methods for Optimizing a Patient's GLP-1 Regimen for Symptom Management Using Continuously Monitored Analyte Data

FIGS. 4A-4B illustrate a flow diagrams of an example methods 400 and 401 for optimizing a GLP-1 medication regimen for a patient to minimize and manage gastrointestinal symptoms based on the patient's glucose and/or lactate levels and providing therapy management guidance to the patient accordingly. For example, therapy management engine 114 can utilize the patient's glucose and/or lactate data, which can be continuously obtained by continuous analyte monitoring system 104, to determine whether the patient's GLP-1 regimen is optimized for achieving the intended effects of the medication (e.g., weight loss), while minimizing the negative side effects (e.g., gastrointestinal symptoms). GLP-1 medications work to decrease the movement of food from the stomach to the small intestine and/or decrease glucose spikes following a meal, all of which are reflected in the patient's analyte data (e.g., glucose and/or lactate data). Therefore, therapy management engine 114 can monitor the patient's glucose and/or lactate data as well as the patient's symptoms to guide the patient to an optimal GLP-1 regimen that is able to achieve the desired decrease in the rate of gastric emptying while minimizing gastrointestinal symptoms and/or health complications that can arise from the GLP-1 regimen. Methods 400 and 401 are described below with reference to FIGS. 1 and 2 and their components.

As discussed above, therapy management engine 114 can use one of a variety of models to determine whether a GLP-1 regimen is optimized for minimizing negative side effects. As described above, the inputs to these models can include glucose and/or lactate data (e.g., received by continuous analyte monitoring system 104), non-analyte data, and/or other patient information (e.g., retrieved from the patient's profile or received via patient inputs) to measure the rate of gastric emptying.

In particular, with respect to minimizing gastrointestinal symptoms, the rate of gastric emptying can be measured during a time period following consumption of a meal (e.g., between 1 minute to 4 hours following a meal) based on an increase in glucose levels and/or other analytes (e.g., lactate, FFAs, or amino acids). For example, subsequent to a meal, an increase in glucose levels can be observed between 15 minutes and 30 minutes following a meal, followed by a decrease in glucose levels as glucose is absorbed and utilized. To measure the rate of gastric emptying, the time of the onset of an increase in glucose levels can be compared to when the meal (e.g., glucose) is consumed and eventually absorbed systemically in the patient's body. Typically, a quick rate of gastric emptying leads to an early peak in blood glucose levels, followed by a decline as glucose is absorbed. However, a slower rate of gastric emptying results in a delayed and possibly lower peak in blood glucose levels, as glucose enters the bloodstream more gradually.

The type of meal, including the form of the meal (e.g., liquid, semisolid, or solid) and the composition of the meal (e.g., glucose alone, glucose with protein, glucose with protein and fat), can influence the time for onset of the increase in glucose levels and, thus, the rate of gastric emptying. For example, a liquid meal with glucose and protein can cause an increase in glucose levels 15 minutes after consumption, followed by a glucose level peak at 30 minutes, and a glucose level return to baseline at 90 minutes. The magnitude of the glucose level peak can be greater when only glucose is consumed. However, when glucose is consumed with protein, for example, the glucose level peak can be decreased by 10-50% or more. In addition, fat co-ingestion with glucose and/or fiber co-ingestion with glucose can cause even greater reductions in glucose level peaks over time, as fat and/or fiber ingestion with glucose has been shown to decrease the rate of gastric emptying, even in the absence of GLP-1 medications. In certain embodiments, if the type of meal is unknown, therapy management engine 114 averages the rate of gastric emptying for longer periods, e.g., one or two weeks, to determine or represent the typical rate of gastric emptying. Alternatively, therapy management engine 114 builds a dynamic histogram of the rate of gastric emptying and monitor the changes of the mean/median of the histogram to determine or represent the chronic changes of the rate of gastric emptying.

In certain embodiments, the rate of gastric emptying can be measured during a time period following consumption of a meal (e.g., between 1 minute to 4 hours following a meal) based on an increase in lactate levels. For example, subsequent to a meal, glucose is converted into lactate and fuels the body before glucose starts to increase after a meal. To measure the rate of gastric emptying, the time of the onset of an increase in lactate levels can be compared to when the meal (e.g., glucose) is consumed. Typically, a fast rate of gastric emptying leads to an early peak in blood lactate levels, followed by a decline in lactate. However, a slower rate of gastric emptying results in a delayed and possibly lower peak in blood lactate levels.

Based on the patient's metrics 132 and/or inputs 130, including meal information, therapy management engine 114 provides specific therapy management guidance to the patient to minimize or manage gastrointestinal symptoms. Additionally, therapy management engine 114 provides positive feedback when the patient's GLP-1 regimen and/or actions (meal times, exercise times, meal content, etc.) are improving the patient's gastrointestinal symptoms. Examples of therapy management guidance can include feedback that the patient's symptoms are getting better over time, recommendations around specific foods to consume based upon past improvement in gastrointestinal symptoms, recommendations around foods that historically caused gastrointestinal symptoms, suggested foods, or combinations of foods to avoid.

FIG. 4A illustrates an example method 400 for optimizing a GLP-1 medication regimen for a patient to minimize and manage gastrointestinal symptoms. Method 400 begins at block 403. At block 403, therapy management engine 114 monitors one or more analyte levels of a patient to monitor the rate of gastric emptying of the patient. In certain embodiments, block 403 is similar to and described in further detail in relation to block 406 of method 401.

At block 405, therapy management engine 114 determines whether the rate of gastric emptying of the patient is decreasing over time. In certain embodiments, block 405 is similar to and described in further detail in relation to block 408 of method 401 in FIG. 4B.

Following block 405, at block 407, therapy management engine 114 determines whether the rate of gastric emptying of the patient meets a first threshold, or whether a reduction in the rate of gastric emptying over a defined period of time meets a second threshold. In certain embodiments, block 407 is similar to and described in further detail in relation to block 410 of method 401 in FIG. 4B.

Following block 407, at block 409, therapy management engine 114 provides therapy management guidance to the patient based on the rate of gastric emptying of the patient. In certain embodiments, block 409 includes therapy management guidance as described in reference to FIG. 4B. For example, the therapy management guidance provided to the patient at block 409 can include the therapy management guidance of block 412. In certain embodiments, the therapy management guidance provided to the patient at block 409 can include the therapy management guidance of block 418. In certain embodiments, the therapy management guidance provided to the patient at block 409 can include the therapy management guidance of block 422. In certain embodiments, the therapy management guidance provided to the patient at block 409 can include the therapy management guidance of block 428. In certain embodiments, the therapy management guidance provided to the patient at block 409 can include the therapy management guidance of block 432. In certain embodiments, the therapy management guidance provided to the patient at block 409 can include the therapy management guidance of block 434.

In another exemplary method, method 401 of FIG. 4B, method 401 can begin at optional block 402 by monitoring one or more analyte levels (e.g., glucose levels, lactate levels, etc.) of a patient to determine one or more analyte metrics. In certain embodiments, monitoring the patient's glucose and/or lactate levels includes determining one or more glucose and/or lactate metrics, such as a glucose rate of change, a glucose minimum or maximum, a lactate rate of change, a lactate minimum or maximum, etc. based on the measured glucose and/or lactate levels. To establish a patient's metrics, therapy management engine 114 monitors the patient's glucose and/or lactate levels over two or more weeks during a pre-medication time period or a post-medication time period.

In certain other embodiments, a patient's metrics are determined based on the patient's glucose and/or lactate levels in response to one or more control meals having certain nutritional compositions consumed at a specific time of day (e.g., morning or evening). For example, the patient can be instructed to consume meals that only include glucose as a liquid or glucose with protein. Alternatively, the patient can be instructed to consume compositions with specifically pre-measured amounts of glucose, protein, fat, fiber, and/or other nutrients in a specific configuration of either a solid, liquid, semisolid, mixture, colloid, or other configuration. Additionally, the patient can be instructed to consume a meal with a specific composition at a specific time of day based on the patient's historical rate of gastric emptying in response to past meals with a known composition.

At block 404, therapy management engine 114 determines whether the patient is prescribed a starting GLP-1 regimen and/or therapy management engine 114 determines and recommends a specific starting GLP-1 regimen for the patient.

In certain embodiments, therapy management engine 114 proceeds to block 404 following optional block 402. In certain other embodiments, if the patient is prescribed a starting GLP-1 regimen, method 401 can begin at block 404. For example, in cases where the patient is not prescribed a starting regimen, therapy management engine 114 determines the patient's starting rate of gastric emptying and recommends a specific starting regimen of GLP-1 medication for the patient, based on the patient's analyte metrics determined at optional block 402. For example, the therapy management engine 114 can use a rules-based model or a machine learning model to use the analyte metrics from optional block 402 to output a recommended starting regimen of GLP-1 medication. In certain embodiments, the therapy management engine 114 can complete the determination of the rate of gastric emptying at block 406 or make a similar determination of the rate gastric emptying as described in block 406 prior to outputting a recommended starting regimen of GLP-1 medication at block 404.

At block 406, therapy management engine 114 monitors one or more analyte levels (e.g., glucose and/or lactate) of the patient to monitor the patient's rate of gastric emptying over time. Based on the patient's analyte levels as determined at block 406, therapy management engine 114 monitors changes in the patient's rate of gastric emptying over time based on the patient's analyte metrics and/or trends over time. In certain embodiments, therapy management engine 114 monitors changes in the patient's rate of gastric emptying over time based on a comparison of the rate of gastric emptying and/or analyte levels of the patient with the rate of gastric emptying and/or analyte levels of a historical patient population on a similar GLP-1 regimen. Alternatively or additionally, the patient's rate of gastric emptying can be monitored by monitoring various analyte metrics of a patient following a specific meal on multiple occasions over time.

At block 408, therapy management engine 114 determines if the rate of gastric emptying of the patient is decreasing over time. In certain embodiments, in order to determine whether the patient's rate of gastric emptying is decreasing over time, therapy management engine 114 first monitors analyte metrics, such as glucose rate of change over time, and/or a lactate rate of change over time to determine the rate of gastric emptying over time.

In particular, as described above, in lieu of direct measurement of the rate of gastric emptying, therapy management engine 114 can derive the rate of gastric emptying over a defined time period from analyte measurements, such as glucose and/or lactate, over the defined time period. This is because the speed at which analyte levels change following consumption of a meal indicates the speed at which gastric material is emptied from the stomach, and thus, the rate of gastric emptying of the patient. There can be at least two approaches for deriving the rate of gastric emptying over a defined time period based on the rate of change of an analyte. These techniques are described below with respect to glucose, however, as described lactate levels may be similarly used. Other analytes may also be measurable and used to infer the rate of gastric emptying in a similar manner.

With respect to the use of glucose measurements to determine a rate of gastric emptying, the first approach involves using the rate of change of glucose, as determined by the therapy management engine 114, as the rate of gastric emptying. For example, if the rate of change of glucose has a value of X mg/dL per minute (mg/dL/min) or X mmol/L per minute (mmol/L/min), then the therapy management engine 114 can use X mg/dL per minute (mg/dL/min) or X mmol/L per minute (mmol/L/min) as the rate of gastric emptying. For example, on day 1, a patient may experience an increase in analyte levels to a specified maximum level 2 hours following a meal, a glucose rate of change of X mmol/L/min may be calculated by therapy management engine 114 while on day 2 the patient may experience an increase in analyte levels to the specified maximum level 3 hours following a similar meal, in which case the therapy management engine 114 may calculate a glucose rate of change of Y mmol/L/min. In such an example, the rate of gastric emptying will be X mmol/L/min on day 1 and Y mmol/L/min on day 2. And, if Y is lower than X, then therapy management engine 114 determines that the rate of gastric emptying is decreasing.

The second approach involves therapy management engine 114 determining the rate of change of glucose and using a mapping to map the rate of change of glucose to a gastric emptying rate. For example, based on empirical studies and research involving historical patient population data, a mapping can be provided that directly correlates various rates of change in glucose to various rates of gastric emptying. In such an example, if on day 1, therapy management engine 114 determines a rate of change of glucose with a value of X mg/dL/min, then the therapy management engine 114 can use the mapping described above to map X mg/dL per minute mg/dL/min to a Y rate of gastric emptying, expressed in amount of volume or food that is still remaining in the stomach over a defined period of time. Then another rate of gastric emptying can be determined on day 2 in a similar manner and the two rates of gastric emptying can be compared and a determination as to whether the rate is decreasing can be made.

In both methods of determining the rate of gastric emptying, the determination of whether the patient's gastric emptying is decreasing over time can be performed over a defined period of time. The defined period of time can be three days, five days, ten days, two weeks, or over a longer or shorter period of time as appropriate. In such an example, therapy management engine 114 can monitor the gastric emptying rate of the patient periodically (e.g., once per hour, once per day) for the defined period of time. If the rate of gastric emptying of the patient over the defined period of time demonstrates a downward trend, therapy management engine 114 determines the rate of gastric emptying of the patient is decreasing. For example, as described above, when the defined period of time is two days, then the rate of gastric emptying on day 2 can be compared with the rate of gastric emptying rate on day 1, and if the former is lower than the latter, then the patient's gastric emptying rate is decreasing over the defined period of time.

However, when the defined period of time is longer and there are a plurality of gastric emptying rates identified over the defined period of time, a downward trend in the gastric emptying rates can be identified with more complex models, such as a linear regression model. If a slope of a linear regression line fit through the rates of gastric emptying is negative, therapy management engine 114 determines that the rate of gastric emptying of the patient is decreasing over time.

If the patient's rate of gastric emptying is not decreasing over time, therapy management engine 114 can continue to block 412. At block 412, therapy management engine 114 continues monitoring the patient's analyte levels. In certain embodiments, therapy management engine 114 provides therapy management guidance to the patient that the patient is not experiencing a decreased rate of gastric emptying. The therapy management guidance can be provided to the patient via a notification, an alert, or an alarm on display device 107. In such a case, therapy management engine 114 can recommend, via the notification, the alert, or the alarm, an increase in dosage of GLP-1.

In some embodiments, in addition to assessing the rate of gastric emptying, therapy management engine 114 can assess other metrics and/or factors. For example, the therapy management engine 114 can assess the amount of weight the patient is losing or if the patient is experiencing other negative side effects. Therapy management engine 114 utilizes these additional metrics, combined with the calculated rate of gastric emptying, to generate an assessment of GLP-1 regimen efficiency. Then, therapy management engine 114 automatically alters the GLP-1 regimen for the patient based on the calculated rate of gastric emptying, the patient's weight loss, the patient's gastrointestinal symptoms, etc. and provides a new GLP-1 regimen to the patient in the form of therapy management guidance. For example, the therapy management engine 114 may automatically alter a dose of the GLP-1 medication, a timing of the GLP-1 medication administration, a frequency of GLP-1 medication administration, and/or a type of GLP-1 medication.

Alternatively, if the rate of gastric emptying of the patient is decreasing over time, therapy management engine 114 proceeds to block 410. At block 410, therapy management engine 114 determines (1) if the patient's rate of gastric emptying meets a first threshold based on the prescribed GLP-1 regimen of the patient and/or (2) if a reduction in the patient's rate of gastric emptying over a defined period of time meets a second threshold.

In the determination of whether the patient's rate of gastric emptying meets the first threshold, the first threshold for the rate of gastric emptying of the patient can be a predefined threshold, X. The first threshold can correspond to the expected rate of gastric emptying (e.g., a particular absolute value) of the patient based on historical data of a rate of gastric emptying of a patient population on a similar GLP-1 regimen. In this example, therapy management engine 114 monitors the rate of gastric emptying of the patient to determine if the rate of gastric emptying of the patient meets or falls below the first threshold, indicating that the patient is likely to experience negative symptoms related to the GLP-1 regimen.

In the determination of whether the reduction in the patient's rate of gastric emptying over a defined period of time meets the second threshold, therapy management engine 114 is configured to evaluate how rapidly the rate of gastric emptying is decelerating. That is because a significant reduction in the rate of gastric emptying is not desirable. As such, the second threshold corresponds to a threshold at or over which any deceleration in the rate of gastric emptying should be identified, such that therapy management guidance can be provided to the patient as described in relation to block 418. For example, therapy management engine 114 can monitor the rate of gastric emptying of the patient over the defined period of time, such as five days. If the reduction in the rate of gastric emptying over the 5 day period exceeds Y units per Z time interval at any point during the five days, therapy management engine 114 determines the reduction in the patient's rate of gastric emptying meets the second threshold.

If either the rate of gastric emptying meets the first threshold or the reduction in the patient's rate of gastric emptying over the defined period of time meets the second threshold, therapy management engine 114 proceeds to block 418. At block 418, therapy management engine 114 provides therapy management guidance to the patient or the patient's caretaker that the patient's GLP-1 regimen can be altered (e.g., the patient's GLP-1 dose can be decreased) to avoid negative side effects and/or health complications. The therapy management guidance can be provided to the patient via a notification, an alert, or an alarm on display device 107.

In addition to or in lieu of adjusting the GLP-1 regimen, for example, if the dosage and/or frequency of the GLP-1 regimen is already at the lowest level possible, or if the intended positive effects are likely to be compromised by lower levels, therapy management engine 114 assesses the regimen adjustment and instead provide alternative solutions. For example, the therapy management engine 114 might determine that instead of the regimen adjustment, the patient can prevent negative side effects and/or reduce the risk of developing negative side effects by implementing one or more alternative solutions, including optimal diet, exercise types, exercise times, and meal times.

In one example, even when GLP-1 regimen can be adjusted, therapy management engine 114 can first monitor if one or more alternative solutions will achieve the desired positive effects of the GLP-1 medication while avoiding negative side effects. In yet another embodiment, a level of GLP-1 regimen adjustment can be combined with alternative solutions to achieve positive effects of the GLP-1 medication to optimize a GLP-1 regimen.

Alternatively, if the patient's rate of gastric emptying does not meet the first threshold and the reduction in the rate of gastric emptying of the patient over the defined period of time does not meet the second threshold, therapy management engine 114 proceeds to block 416. At block 416, therapy management engine 114 monitors the patient's reported digestive symptoms (e.g., nausea, constipation, vomiting, diarrhea, etc.) to determine whether the patient is experiencing negative digestive symptoms related to the patient's GLP-1 regimen or otherwise. While the below steps starting from block 416 can help further optimize the regimen of GLP-1 medications, in some examples or in certain situations, these steps can be optional and/or not performed. In such embodiments, after the assessment that the rate of gastric emptying is not below the expected threshold, the method 401 returns to block 402 to continue monitoring the analyte levels of the patient.

At block 420, therapy management engine 114 determines whether the patient is experiencing digestive symptoms. If the patient is not experiencing digestive symptoms, therapy management engine 114 proceeds to block 422. At block 422, therapy management engine 114 provides therapy management guidance to the patient regarding optimal diet, exercise, and meal times to prevent symptoms while continuing to monitor the patient's analyte metrics and/or rate of gastric emptying over time. The therapy management guidance can be provided to the patient via a notification, an alert, or an alarm on display device 107.

In certain embodiments, if the patient does not experience digestive symptoms at the prescribed regimen of GLP-1 over time and the patient is not experiencing weight loss and/or changes in analyte metrics following a meal, therapy management engine 114 instructs the patient to titrate up the GLP-1 dose of the GLP-1 regimen at a certain time period (e.g., increase GLP-1 dose slightly every 4 weeks) or increase the frequency of the GLP-1 regimen.

Alternatively, if therapy management engine 114 determines that the patient is experiencing digestive symptoms, therapy management engine 114 proceeds to block 424. At block 424, therapy management engine 114 determines whether the patient is experiencing severe digestive symptoms. In certain embodiments, the determination of whether the patient is experiencing severe digestive symptoms is based on whether the digestive symptoms of the patient are above or below a threshold of expected digestive symptoms based on the prescribed GLP-1 dose of the patient (e.g., when compared to historical data of digestive symptoms of a patient population taking a similar GLP-1 dose, or based on historical patient data). If the patient's digestive symptoms are severe, therapy management engine 114 proceeds to block 428. At block 428, therapy management engine 114 provides therapy management guidance to the patient to seek medical intervention immediately for a potential health complication (e.g., an intestinal blockage). The therapy management guidance can be provided to the patient via a notification, an alert, or an alarm on display device 107.

Alternatively, if the patient's digestive symptoms are not severe, therapy management engine 114 proceeds to block 426. At block 426, therapy management engine 114 monitors the patient's symptoms over time (e.g., over two weeks) to determine if the patient's symptoms resolve on their own. In certain embodiments, based on the patient's analyte metrics, the therapy management system provides a prediction to the patient that the patient may experience symptoms for a time period (e.g., one week) but that the patient's symptoms will resolve over time.

At block 430, therapy management engine 114 determines whether the patient's digestive symptoms resolve over time (e.g., within two weeks). For example, therapy management engine 114 determines whether the patient's digestive symptoms resolve over time by monitoring each meal and determining whether there is an improvement in the rate of gastric emptying. Conversely, therapy management engine 114 could predict that symptoms will worsen based on monitoring the rate of gastric emptying for each meal and comparing to the previous. If the patient's symptoms resolve over two weeks, therapy management engine 114 proceeds to block 432. At block 432, therapy management engine 114 provides therapy management guidance to the patient or the patient's caretaker that the patient's GLP-1 regimen can be maintained and therapy management engine 114 can continue monitoring the patient's analyte metrics and the rate of gastric emptying over time. The therapy management guidance can be provided to the patient via a notification, an alert, or an alarm on display device 107. Further, because a GLP-1 dosage of the GLP-1 regimen is typically titrated up at a known schedule, therapy management engine 114 could suggest when a new dosage could be tolerated and how long the side effects will persist once the new higher dosage is taken.

Alternatively, if the patient's digestive symptoms do not resolve over time, therapy management engine 114 proceeds to block 434. At block 434, therapy management engine 114 provides therapy management guidance to the patient on decreasing GLP-1 dose or frequency of the GLP-1 regimen, optimal diet, exercise times, meal times, etc. to increase the rate of gastric emptying (e.g., decrease stomach retention) and/or provide guidance to the patient related to digestive symptoms to reduce or resolve digestive symptoms. The therapy management guidance can be provided to the patient via a notification, an alert, or an alarm on display device 107. Following the therapy management guidance at block 434, the method returns to block 406 to continue monitoring the analyte levels of the patient.

In certain embodiments, meal time recommendations provided by therapy management engine 114 can be related to the timing of the meal time, the meal type, and/or the size and nutritional contents of the meal relative to the GLP-1 regimen of the patient. For example, if the patient is taking a GLP-1 medication via weekly injections, therapy management engine 114 instructs the patient to eat different types of food based on how recently the patient injected their GLP-1 dose (e.g., the patient just injected their GLP-1 dose that day as opposed to the day before the patient's weekly injection (e.g., six days following the injection)).

In certain embodiments, an optimal diet recommendation provided by therapy management engine 114 can be based on how different foods or food preparations impact the patient's rate of gastric emptying, therefore causing gastrointestinal symptoms. For example, fried foods, high fat foods, alcohol, and other specific nutritional components of food can decrease the rate of gastric emptying and increase stomach retention. Therefore, patients on GLP-1 medications struggling with decreased rates of gastric emptying can be instructed to avoid these types and/or preparations of food. Additionally, therapy management engine 114 can provide one or more other diet recommendations including eating more frequent, smaller meals, reducing fat intake, reducing fiber intake, reducing alcohol consumption, and/or supplementing solid meals with liquid meal consumption.

While glucose and lactate metrics are discussed in reference to FIGS. 4A-4B to determine when the patient's rate of gastric emptying is below the threshold, additional analytes described herein can be utilized to determine when a patient is suffering from a more severe health complication. In certain embodiments, therapy management engine 114 monitors FFAs, glycerol, monoglycerides, and cholesterol levels following the consumption of a meal with a known level of fats. Based on the meal having a known level of fats, therapy management engine 114 approximates bile excretion and subsequent absorption (e.g., timing or amount of absorption) of FFAs, glycerol, monoglycerides, and/or cholesterol to alert the patient or the patient's caretaker of signs of biliary dysfunction. For example, patients with biliary and/or gall bladder dysfunction can experience a change in timing and amount of absorption of FFAs, glycerol, monoglycerides, and cholesterol when compared to expected absorption values.

Additionally, amino acid levels (e.g., from protein consumption) and lactate levels (e.g., from lactate or fructose consumption) can increase as they are absorbed following a meal and allow for a similar determination of the rate of gastric emptying. Unlike glucose levels, amino acid levels and lactate levels are not impacted as significantly by GLP-1 medication and, therefore, these analytes enable an accurate determination of the rate of gastric emptying. Therefore, one or more of these analytes can be used as described in FIGS. 4A-4B, such that various analyte metrics (e.g., the time of onset of increase in the analyte level, peak analyte level, and time to return to at and/or near pre-prandial analyte levels) can be utilized to determine and monitor the rate of gastric emptying.

Further, when monitoring for more severe health complications, therapy management engine 114 can monitor one or more of calcitonin, thyroid stimulating hormone (TSH), triiodothyronine (T3), thyroxine (T4), thyroglobulin (Tg), and/or thyroid peroxidase (TPO) levels in combination with glucose data and the rate of gastric emptying to determine a risk or presence of thyroid cancer. For example, if the parafollicular cells (C-cells) of a patient's thyroid are producing too much calcitonin, the patient's calcitonin levels will increase, which can be indicative of medullary thyroid cancer or C-cell hyperplasia. To prevent the development of thyroid cancer and/or other thyroid complications, therapy management engine 114 monitors the patient's calcitonin levels to ensure calcitonin levels do not increase as that patient continues to take GLP-1 medications. If, however, therapy management engine 114 determines the patient is experiencing an increase in calcitonin levels over time, therapy management engine 114 recommends the patient seek medical intervention for potential thyroid dysfunction. In certain embodiments, the patient can be prompted to wear and/or provided with a calcitonin sensor during certain time periods when the patient's glucose and/or other analyte metrics deviate from the expected baseline and/or other metrics. For example, a patient can be instructed to wear a calcitonin sensor for a few weeks per year to identify and treat potential thyroid dysfunction and/or thyroid cancers.

While the method 401 described herein provides therapy management guidance to patients for managing negative gastrointestinal symptoms of GLP-1 medications, the systems and methods provided herein can also provide therapy management guidance to patients taking various other medications that decrease the rate of gastric emptying, cause gastrointestinal symptoms, and/or cause gastroparesis. For example, the following is a list of example medications that are known to slow the rate of gastric emptying in at least some circumstances: Anticholinergic medications (Diphenhydramine, Tricyclic Antidepressants), Calcium Channel Blockers, Opiates (Morphine, Codeine), Tricyclic Antidepressants (Nortriptyline, Amitriptyline), Dopamine Agonists. In addition, the following are a list of medications that are known to speed up gastric motility: Prokinetic agents (Domperidone, Erythromycin), Cisapride, Bethanechol, Prucalopride (selective 5-hydroxytryptamine 4 receptor agonist), and caffeine. In addition, the concentration of some substances (e.g., alcohol) can be used to determine if the patient is likely to experience decreased rates of gastric emptying or accelerated rates of gastric emptying. Specifically, alcohol in high concentrations (e.g., greater than 15 percent) decreases the rate of gastric emptying, while drinks with low alcohol concentrations (e.g., less than 15 percent, most wine or beer) increase the rate of gastric emptying. The above list of example medications and substances is exemplary and not a comprehensive list of all medications that can have an effect on the rate of gastric emptying and gastric motility. In addition, some medical devices and therapies, such as, but not limited to, nerve stimulation, nasal or other types of gastric tubes, and CPAP devices can impact the rate of gastric emptying.

Further, systems and methods described herein can also provide therapy management guidance to patients with diabetes and/or obese patients experiencing or at risk of experiencing gastrointestinal symptoms to monitor, minimize, and/or treat gastrointestinal symptoms and/or gastroparesis.

### Example Methods for Optimizing a Patient's GLP-1 Regimen for Weight Loss and Maintaining Weight Loss Using Continuously Monitored Analyte Data

FIG. 5A illustrates an example method 500 for providing therapy management guidance for optimizing GLP-1 regimen for weight loss and to maintain weight loss based on a patient's glucose and/or lactate levels. Therapy management engine 114 utilizes the patient's glucose and/or lactate data, monitored over time by continuous analyte monitoring system 104, to determine whether the patient's GLP-1 regimen is optimized for the patient's weight loss. Method 500 is described below with reference to FIGS. 1 and 2 and their components.

Method 500 of FIG. 5A begins at block 503. At block 503, therapy management engine 114 monitors one or more analyte levels of a patient, the one or more analyte levels including at least glucose levels and lactate levels. In certain embodiments, block 503 is similar to and described in further detail in relation to block 506 of FIG. 5B.

At block 505, based on the analyte levels of the patient, therapy management engine 114 determines an expected weight loss of the patient. In certain embodiments, block 505 is similar to and described in further detail in relation to block 508 of method 501 of FIG. 5B.

Following block 505, at block 507, therapy management engine 114 determines whether the patient has reached their weight loss goal. In certain embodiments, block 507 is similar to and described in further detail in relation to block 512 of method 501 of FIG. 5B.

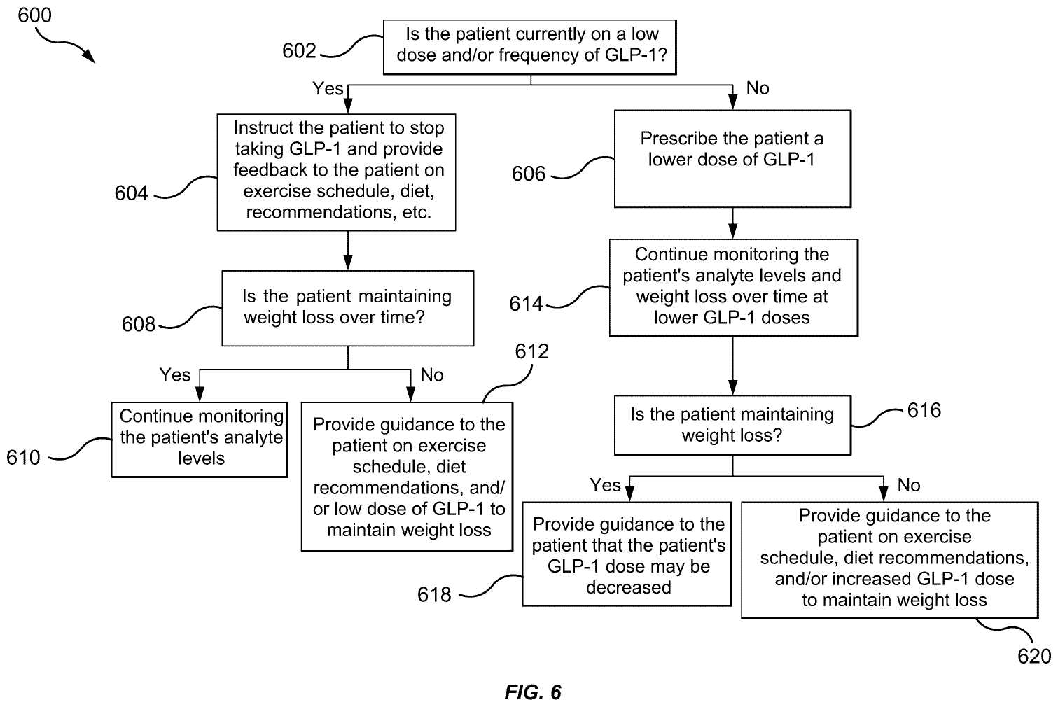

Following block 507, at optional block 509, therapy management engine 114 determines whether the patient is maintaining weight loss over time. In certain embodiments, block 509 is similar to and described in further detail in relation to block 608 of method 600 of FIG. 6. In certain embodiments, block 509 is similar to and described in further detail in relation to block 616 described in reference to FIG. 6.

Following block 509, at block 511, therapy management engine 114 provides therapy management guidance to the patient based on the expected weight loss of the patient and/or whether the patient has reached their weight loss goal. In certain embodiments, block 511 includes therapy management guidance as described in reference to FIGS. 5B and 6. For example, the therapy management guidance provided to the patient at block 511 can include the therapy management guidance of block 522. In certain embodiments, the therapy management guidance provided to the patient at block 511 can include the therapy management guidance of block 526. In certain embodiments, the therapy management guidance provided to the patient at block 511 can include the therapy management guidance of block 532. In certain embodiments, the therapy management guidance provided to the patient at block 511 can include the therapy management guidance of block 534. In certain embodiments, the therapy management guidance provided to the patient at block 511 can include the therapy management guidance of block 526. In certain embodiments, the therapy management guidance provided to the patient at block 511 can include the therapy management guidance of block 612. In certain embodiments, the therapy management guidance provided to the patient at block 511 can include the therapy management guidance of block 618. In certain embodiments, the therapy management guidance provided to the patient at block 511 can include the therapy management guidance of block 620.

FIG. 5B illustrates an example method 501 for providing therapy management guidance for optimizing GLP-1 regimen for weight loss based on a patient's glucose and/or lactate levels. Therapy management engine 114 utilizes the patient's glucose and/or lactate data, monitored over time by continuous analyte monitoring system 104, to determine whether the patient's GLP-1 regimen is optimized for the patient's weight loss. Method 501 is described below with reference to FIGS. 1 and 2 and their components.

Method 501 of FIG. 5B can begin optional at block 502 by monitoring one or more analyte levels (e.g., glucose and/or lactate levels) of a patient to determine one or more glucose and/or lactate metrics. In certain embodiments, monitoring the patient's glucose and/or lactate metrics includes determining a glucose rate of change, a glucose minimum or maximum, timing of a glucose minimum or maximum, a lactate rate of change, a lactate minimum or maximum, timing of a lactate minimum or maximum, etc. based on the measured glucose and/or lactate levels. To establish a patient's metrics, therapy management engine 114 monitors the patient's glucose and/or lactate levels over two or more weeks during a pre-medication time period or a post-medication time period.

At block 504, therapy management engine 114 determines whether the patient is prescribed a starting dose and frequency of GLP-1 medication and/or recommends a specific starting GLP-1 regimen for the patient. In cases where the patient is not prescribed a starting dose, the therapy management engine 114 recommends a specific starting dose of GLP-1 medication for the patient, based on the patient's glucose and/or lactate metrics monitored at optional block 502. For example, the therapy management engine 114 uses a rules-based model or a machine learning model to use the glucose and/or lactate metrics from block 502 to output a recommended starting dose of GLP-1 medication. In some examples, the determination at block 504 is based, at least in part, on the monitoring at block 502. However, in certain embodiments, other data inputs can lead to the determination of a starting dose of GLP-1 medication at block 504. Where other inputs and/or historical analyte data is used to determine GLP-1 medication dosage, the monitoring at block 502 is effectively replaced by block 506.

At block 506, therapy management engine 114 monitors one or more analyte level levels (e.g., glucose and/or lactate levels) of the patient over time. At block 508, therapy management engine 114 determines, at least based on the patient's glucose and/or lactate levels over time and/or adherence to the GLP-1 regimen, the patient's expected weight loss. For example, the patient's expected weight loss can be determined based on various glucose and/or lactate levels and/or metrics following a meal and/or adherence to the prescribed GLP-1 regimen. In certain embodiments, the glucose and/or lactate levels and/or metrics include glucose time in range, timing and magnitude of glucose peaks, rate of change of glucose levels (increasing or decreasing), and/or duration of glucose elevation following a meal. In certain embodiments, a rules-based model can be used to determine the expected weight loss of the patient based on these metrics. In one example of a rules-based model, these metrics can be compared to the patient's corresponding baseline metrics, based on which comparison an expected weight loss can be determined using one or more rules of the rules-based models.

As described above, the rules-based model can further take into account the patient's compliance with a GLP-1 regimen. For example, the patient can be instructed to consume a meal to monitor the amount of time it takes for the patient's glucose levels to spike (e.g., post-prandial spike) and return to baseline. A patient taking GLP-1 medications regularly (e.g., as prescribed) would experience a lower post-prandial glucose peak and a decreased duration of glucose elevation following a meal. Therefore, the lower post-prandial glucose peak and time to return to baseline indicate that the patient is compliant with their GLP-1 regimen and can expect to see similar weight loss as other compliant patients on a similar GLP-1 regimen.

In certain embodiments, the therapy management engine 114 requests that the patient perform a meal test to determine the patient's baseline gastroparesis activity. The meal test can include a controlled meal with known macronutrient quantities of solid, liquid, or mixed meal (e.g., solid and liquid). A controlled meal can include an OGTT (oral glucose tolerance test) or perhaps a drink (e.g., “Boba drink”) where there are solids and liquids of similar nutritional content that could be used in measuring the time of ingestion and the rate of gastric emptying as a way to get baseline gastroparesis activity. Further, therapy management engine 114 could also use this kind of meal test with and without GLP-1 and compare the rate of gastric emptying in response, as measured by glucose and/or lactate data, against a baseline response to see how the GLP-1 affects the emptying time of the materials. The therapy management engine 114 can then provide recommendation as to the dosage amounts and frequency of the GLP-1 medication based on the comparison above.

In some examples, the meal test can be specifically prescribed to the patient and then the patient's glucose and/or lactate levels can be monitored. In other examples, the patient can proceed to eat regularly, and the therapy management engine 114 can perform the meal test by automatically selecting specific patterns that correspond to specific detected meal types without needing to prescribe a specific meal regimen to the patient. For example, in lieu of an OGTT, the therapy management engine 114 can look for similar foods regularly eaten by the patient that cause similar changes (perhaps of different magnitudes) and can use those foods to perform the meal test described above.

In certain embodiments, in addition to the patient's analyte data and/or compliance with the GLP-1 regimen, a patient's biographic information, treatment or medication information (e.g., steroids, cholesterol lowering medications, NSAIDS, etc.), medical history, and/or disease diagnoses (e.g., liver disease, cancer, kidney disease, COPD, obesity, diabetes, etc.) are taken into account by the rules-based model when determining the patient's expected weight loss. For example, patients who report taking steroids can experience periods of hyperglycemia that are more pronounced than an average patient and, therefore, the rules-based model can be configured to account for the patient's use of steroids in determining an expected weight loss based on the patient's glucose rate of change, for example. In another example, healthy older individuals can experience delayed glucose level spikes (e.g., from 30 minutes in healthy younger individuals to 45 minutes in older individuals). Additionally, healthy younger individuals can return to baseline glucose levels more quickly, e.g., 90 minutes, when compared to healthy older individuals who can experience a return to baseline glucose levels at 120 minutes.

Disease diagnoses can also affect glucose regulation as patients with later stages of chronic kidney disease can have more pronounced glucose swings, especially patients on dialysis. Similarly, patients with liver disease can experience a higher baseline glucose level and a slower return to baseline glucose levels as compared to similar patients without liver disease. An obese or diabetic patient can also experience decreased rates of gastric emptying (e.g., delayed glucose level maximums). As such, the rules-based model can be configured with rules around one or more of the additional factors described above.

At block 510, therapy management engine 114 determines whether the patient's expected weight loss (e.g., losing X pounds in Y days or weeks and/or losing Z percentage of the patient's body weight in Y days or weeks) is within a predefined threshold of a population-based expected weight loss derived from historical patient population data. More specifically, the population-based expected weight loss refers to the expected weight loss of a historical patient population on a similar GLP-1 regimen experienced.

If the patient's expected weight loss is within the predefined threshold of the population-based expected weight loss based on the patient's GLP-1 regimen, therapy management engine 114 proceeds to block 512. At block 512, therapy management engine 114 determines whether the patient has reached their weight loss goal. In some examples, instead of or in addition to population based data, a personalized weight loss goal can be generated for the patient using the information provided above. The personalized expected weight loss goal can be used to assess the expected weight loss is consistent with expected weight loss goal (i.e., the personalized weight loss expectation).

If the patient has not reached their weight loss goal, therapy management engine 114 proceeds to block 514. At block 514, therapy management engine 114 continues monitoring the patient's glucose and/or lactate levels and non-analyte data (e.g., weight information) to determine if the patient reaches their weight loss goal over time. Specifically, the patient's lactate levels and metrics (e.g., baseline lactate level, lactate rate of change, etc.) over time can demonstrate an improvement in the patient's muscle mitochondrial health and liver health. For example, if the patient experiences lower baseline lactate levels and/or increased lactate rate of change following an increase in lactate levels (e.g., increased lactate clearance rate), then the patient's muscle mitochondrial health and liver health is improving. Improvement in muscle mitochondrial health and liver health indicates that the patient is progressing towards a weight loss goal and the patient may be able to begin decreasing their GLP-1 medication dosage without a risk of weight gain. Alternatively, if the patient has reached their weight loss goal, therapy management engine 114 proceeds to block 516. At block 516, therapy management engine 114 proceeds to FIG. 6 described below.

However, if the patient's weight loss is not within a predefined threshold of the population-based expected weight loss, therapy management engine 114 proceeds to block 518. At block 518, therapy management engine 114 monitors the patient's reported gastrointestinal symptoms over time to evaluate the undesired effects of the GLP-1 medication dose on the patient. For example, the patient can regularly be asked to report any symptoms experienced subsequent to taking the GLP-1 medication. Block 518 and subsequent blocks described herein are not required to be performed if the dosage can be increased in a way that would not risk negative side effects, or if the benefits of increased effectiveness of the therapy outweigh the negative side effects. In some examples, block 520-534 can be performed separately on a continuous basis and not following the assessment of negative side effects of the GLP-1 regimen.

Based on the monitoring at block 518, at block 520, therapy management engine 114 determines whether the patient is experiencing gastrointestinal symptoms. If the patient is not experiencing gastrointestinal symptoms, therapy management engine 114 proceeds to block 522. At block 522, therapy management engine 114 provides therapy management guidance to the patient on optimal diet, exercise, and/or meal times to minimize any future gastrointestinal symptoms. The therapy management guidance can be provided to the patient via a notification, an alert, or an alarm on display device 107. Further, therapy management engine 114 provides therapy management guidance to the patient that encourages compliance with the patient's current GLP-1 regimen. Therapy management engine 114 then returns to block 508 to continue monitoring the patient's analyte levels to determine expected weight loss.

Alternatively, if the patient is experiencing gastrointestinal symptoms, therapy management engine 114 proceeds to block 524. At block 524, therapy management engine 114 determines whether the patient is experiencing severe gastrointestinal symptoms. For example, the patient can be asked to report the severity of the symptoms. If the patient is experiencing severe gastrointestinal symptoms, therapy management engine 114 proceeds to block 526. At block 526, therapy management engine 114 provides therapy management guidance to the patient to seek medical intervention for a potential urgent health complication, such as an intestinal blockage. The therapy management guidance can be provided to the patient via a notification, an alert, or an alarm on display device 107.

Alternatively, if the patient is not experiencing severe gastrointestinal symptoms, therapy management engine 114 proceeds to block 528. At block 528, therapy management engine 114 monitors the patient's gastrointestinal symptoms over a particular time period (e.g., two weeks). For example, therapy management engine 114 can regularly ask the patient to report their symptoms over the time period. At block 530, therapy management engine 114 determines whether the patient's gastrointestinal symptoms subside over the time period. If the patient's gastrointestinal symptoms subside over the time period, therapy management engine 114 proceeds to block 532. At block 532, therapy management engine 114 provides therapy management guidance to the patient or the patient's caretaker that the patient's GLP-1 regimen can be altered (e.g., the patient's GLP-1 dose can be increased) to optimize the patient's weight loss. The therapy management guidance can be provided to the patient via a notification, an alert, or an alarm on display device 107. Then, therapy management engine 114 returns to block 506 to continue monitoring the patient's analyte levels.

Alternatively, if the patient's gastrointestinal symptoms do not subside over the time period, therapy management engine 114 proceeds to block 534. At block 534, therapy management engine 114 provides therapy management guidance to the patient relating to decreasing the prescribed GLP-1 dosage of the GLP-1 regimen, lifestyle changes, diet, medication timing, etc. to optimize weight loss and/or resolve symptoms. The therapy management guidance can be provided to the patient via a notification, an alert, or an alarm on display device 107. Therapy management engine 114 then similarly returns to block 506 to continue monitoring the patient's analyte levels.

In certain embodiments, the goal of the GLP-1 medication is not just weight loss but more generally increased metabolic health, kidney health, or liver health. In such examples, instead of just assessing weight loss, the efficacy of the GLP-1 regimen is determined based on expected positive effects of the GLP-1 medication. In some examples, the expected positive effects can be weight loss generally, or therapy management engine 114 can assist the patient in specifically losing a specific amount of fat but avoiding loss of muscle while taking a GLP-1 medication. Furthermore, in lieu of weight loss alone, therapy management engine 114 can monitor whether the patient has achieved a goal related to the patient's fat to muscle ratio. Still further, therapy management engine 114 can monitor whether the patient has achieved a goal related to an increased fitness or improved glucose and/or lactate clearance rate. For example, therapy management engine 114 recommends a specific diet and/or exercise (e.g., consume more protein and/or complete Zone 2 exercise) in order to assist the patient in retaining muscle while losing weight. Further, therapy management engine 114 monitors lactate and/or creatinine levels to determine how often the patient exercises, the type of exercises the patient engages in, etc. For example, short term increases in lactate levels indicate the patient is exercising, which can be indicative of retaining muscle mass. Over time, if the patient produces less lactate while exercising, the patient is retaining and/or improving muscle mass and muscle fitness. Alternatively, if the patient experiences increasing lactate levels over time during exercise, therapy management engine 114 determines the patient is losing muscle.

Further, creatinine levels can be used to monitor a user's kidney function and/or kidney disease over time. For example, if the user has a high creatinine level and experiences a reduction in creatinine levels to a normal range over time, therapy management engine 114 determines that the user's kidney function and/or kidney disease is improving. However, even if the user's kidney function is improving, the user's creatinine levels may not reach a healthy range for months and/or years.

In addition to being used to determine kidney function, creatinine levels are directly correlated with muscle mass. For example, a reduction in creatinine levels indicate the patient is losing muscle over time. Additionally, therapy management engine 114 monitors the patient's VO2 max (e.g., the amount of oxygen the patient's body uses while exercising) and/or the patient's grip strength via a grip strength test to further determine when the patient is losing muscle. Therapy management engine 114 assists the patient in losing weight through fat loss while maintaining weight loss over time, as described in more detail in FIG. 6.

FIG. 6 illustrates a flow diagram of an example method 600 for providing therapy management guidance to a patient for maintaining the positive effects of the GLP-1 medication over time while titrating down and/or completely stopping GLP-1 medication. For example, the example method 600 is described in relation to providing therapy management guidance for maintaining weight loss while titrating down and/or completely stopping GLP-1 medication. The method 600 can be performed in response to a determination (e.g., based on patient input or other means) that the patient is currently on a GLP-1 regimen including a low dose and/or frequency of GLP-1, the patient is currently altering or stopping their GLP-1 regimen, or the patient has reached their weight loss goal based on patient input or as described in reference to block 516 of FIG. 5. Although method 600 is described in relation to providing therapy management guidance to a patient for maintaining weight loss, the flow diagram similarly encompasses the provision of therapy management guidance to a patient for maintaining other positive effects of the GLP-1 medication, including metabolic fitness, fat to muscle ratio, fitness score improvement, liver health, kidney health, glucose clearance improvement, improvement in insulin resistance, etc.

Method 600 begins at block 602 by therapy management engine 114 determining whether the patient is currently on a low dose and/or frequency of GLP-1 medication.

If the patient is currently on a low dose and/or frequency of GLP-1 medication, therapy management engine 114 proceeds to block 604. At block 604, therapy management engine 114 instructs the patient to stop taking GLP-1 and provides therapy management guidance to the patient in relation to exercise, exercise schedule, diet, etc. to assist the patient in maintaining weight loss. At block 608, therapy management engine 114 determines, based on analyte data and/or patient input, whether the patient is maintaining weight loss over time (e.g., two weeks). If the patient is maintaining weight loss over time, therapy management engine 114 proceeds to block 610. At block 610, therapy management engine 114 continues monitoring a patient's analyte levels as described in block 608 to evaluate the patient's weight loss over time. If the patient is not maintaining weight loss over time, therapy management engine 114 proceeds to block 612. At block 612, therapy management engine 114 provides therapy management guidance to the patient relating to exercise, exercise schedule, diet recommendations and/or resuming a low dose of GLP-1 in order to maintain weight loss. The therapy management guidance can be provided to the patient via a notification, an alert, or an alarm on display device 107.

Alternatively, if the patient is not determined to be on a low dose and/or frequency of GLP-1 at block 602 and has reached their weight loss goal and/or otherwise provided input to therapy management engine 114 that they would like to stop or decrease their GLP-1 regimen, therapy management engine 114 proceeds to block 606. At block 606, therapy management engine 114 alters the patient's GLP-1 regimen. For example, therapy management engine 114 prescribes the patient a lower dose of GLP-1 (e.g., decrease the dose amount and/or dose frequency) and/or prescribes the patient an alternative GLP-1 medication. Once the patient is on the altered GLP-1 regimen, therapy management engine 114 proceeds to block 614. At block 614, therapy management engine 114 continues monitoring the patient's analyte levels and weight loss over time.

At block 616, therapy management engine 114 determines whether the patient is maintaining weight loss over time based on the patient's analyte levels and/or reported weight loss. As described herein, alternatively or additionally to providing weight loss guidance, therapy management engine 114 can determine whether the patient is maintaining one or more other positive effects of the GLP-1 medication. In certain embodiments, this determination is based on one or more analyte levels of the patient, including lactate clearance rates, lactate baseline levels, 02 levels, glucose clearance levels, potassium levels, creatinine levels, glucose level spikes, glucose variability, etc.

If the patient is maintaining weight loss, therapy management engine 114 proceeds to block 618. At block 618, therapy management engine 114 provides therapy management guidance to the patient or the patient's caretaker that the patient's GLP-1 regimen can be decreased or discontinued. The therapy management guidance can be provided to the patient via a notification, an alert, or an alarm on display device 107. Therapy management engine 114 continues monitoring the patient's analyte metrics and/or weight loss over time.

Alternatively, if the patient is not maintaining weight loss over time, therapy management engine 114 proceeds to block 620. At block 620, therapy management engine 114 provides therapy management guidance to the patient on exercise schedule, diet recommendations, and/or increased GLP-1 dose to maintain weight loss over time. The therapy management guidance can be provided to the patient via a notification, an alert, or an alarm on display device 107. Following the therapy management guidance at block 620, the method returns to block 602 to continue monitoring the GLP-1 regimen of the patient to assist the patient in altering the GLP-1 regimen while maintaining weight loss and/or other positive effects of the GLP-1 medication.

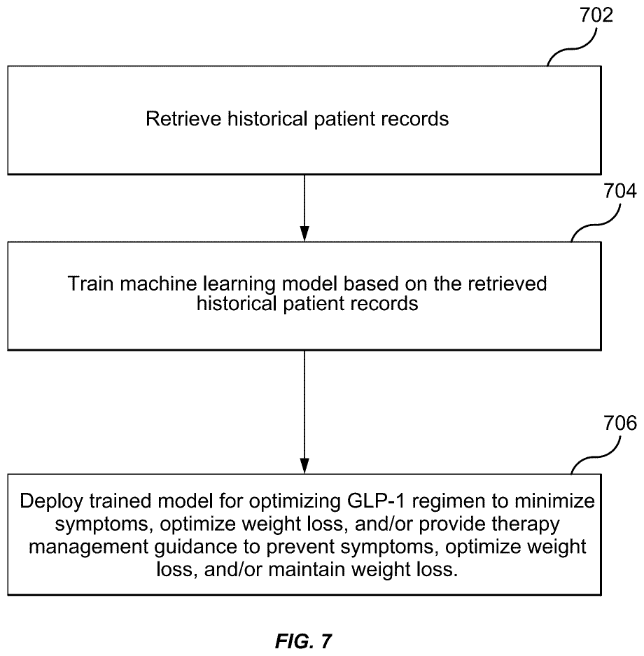

FIG. 7 is a flow diagram depicting a method 700 for training machine learning models for optimizing a patient's GLP-1 regimen to minimize gastrointestinal symptoms and/or optimize weight loss. Alternatively or additionally, one or more of the machine learning models can be trained to provide therapy management guidance on medication parameters, specific diet recommendations, meal times, and/or exercise regimens to minimize symptoms, optimize weight loss, and/or maintain weight loss.

Method 700 begins, at block 702, by training server system, such as training server system 140 illustrated in FIG. 1, retrieving data from historical records database, such as historical records database 112 illustrated in FIG. 1. As mentioned herein, historical records database 112 can provide a repository of up-to-date information and historical information for patients of a continuous analyte monitoring system and connected mobile health application, such as patients of continuous analyte monitoring system 104 and application 106 illustrated in FIG. 1, as well as data for one or more patients who are not, or were not previously, patients of continuous analyte monitoring system 104 and/or application 106. In certain embodiments, historical records database 112 can include one or more data sets of historical patients who followed various GLP-1 regimens, reached or did not reach various weight loss goals, experienced or did not experience various gastrointestinal symptoms, and/or the like.

Retrieval of data from historical records database 112 by training server system 140, at block 702, can include the retrieval of all, or any subset of, information maintained by historical records database 112. For example, where historical records database 112 stores information for 100,000 patients (e.g., non-users and users of continuous analyte monitoring system 104 and application 106), data retrieved by training server system 140 to train one or more machine learning models can include information for all 100,000 patients or only a subset of the data for those patients, e.g., data associated with only 50,000 patients or only data from the last ten years.

As an illustrative example, integrating with on-premises or cloud-based medical record databases through Fast Healthcare Interoperability Resources (FHIR), web application programming interfaces (APIs), Health Level 7 (HL7), and/or other computer interface language can enable aggregation of healthcare historical records for baseline assessment in addition to the aggregation of de-identifiable patient data from a cloud-based repository. Similarly, when integrating into the medical record databases, the integration can be accomplished by directly interfacing with the electronic medical record system or through one or more intermediary systems (e.g., an interface engine, etc.).

As an illustrative example, at block 702, training server system 140 can retrieve information for 100,000 patients with various states (e.g., healthy patient, patients taking various doses of GLP-1 medication, patients who have reached various weight loss goals, and/or a patients experiencing various symptoms) stored in historical records database 112 to train a model to optimize a GLP-1 regimen and provide recommendations to the patient. Each of the 100,000 patients can have a corresponding data record (e.g., based on their corresponding patient profile), stored in historical records database 112. Each patient profile 118 can include information, such as information discussed with respect to FIG. 3.

The training server system 140 then uses information in each of the records to train an artificial intelligence or ML model (for simplicity referred to as “ML model” herein). Examples of types of information included in a patient's profile were provided above. The information in each of these records can be featurized (e.g., manually or by training server system 140), resulting in features that can be used as input features for training the ML model. For example, a patient record can include or be used to generate features related to the patient's demographic information (e.g., an age of a patient, a gender of the patient, etc.), analyte information, such as lactate and glucose metrics, non-analyte information, and/or any other data points in the patient record (e.g., inputs 130, metrics 132, etc.). Features used to train the machine learning model(s) can vary in different embodiments.

In certain embodiments, each historical patient record retrieved from historical records database 112 is further associated with a label indicating the corresponding patient's GLP-1 regimen, the patient's gastrointestinal symptoms, the patient's weight loss information, etc. What the record is labeled with would depend on what the model is being trained to predict.

At block 704, method 700 continues by training server system 140 training one or more machine learning models based on the features and labels associated with the historical patient records. In some embodiments, the training server does so by providing the features as input into a model. This model can be a new model initialized with random weights and parameters, or can be partially or fully pre-trained (e.g., based on prior training rounds). Based on the input features, the model-in-training generates some output. In certain embodiments, the output can include a prediction of a patient's weight loss based on the GLP-1 regimen, a diagnosis of one or more gastrointestinal symptoms, and/or recommendations for medication parameters, specific diet recommendations, meal times, and/or exercise regimens to minimize symptoms, optimize weight loss, and/or maintain weight loss, or similar outputs. Note that the output could be in the form of a determination, a recommendation, and/or other types of output.

In certain embodiments, training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict a patient's weight loss based on the GLP-1 regimen, a diagnosis of one or more gastrointestinal symptoms, and/or recommendations to minimize symptoms, optimize weight loss, and/or maintain weight loss more accurately.

One of a variety of machine learning algorithms can be used for training the model(s) described above. For example, one of a supervised learning algorithm, a neural network algorithm, a deep neural network algorithm, a deep learning algorithm, etc. can be used.

At block 706, training server system 140 deploys the trained model(s) to make predictions associated with optimizing GLP-1 regimen for a patient to minimize gastrointestinal symptoms, optimize weight loss of a patient, and/or the like. In certain embodiments, the trained model can be configured to provide therapy management recommendations for medication parameters, specific diet recommendations, meal times, and/or exercise regimens to minimize symptoms, optimize weight loss, and/or maintain weight loss during runtime. In some embodiments, deploying the model includes transmitting some indication of the trained model(s) (e.g., a weights vector) that can be used to instantiate the model(s) on another device. For example, training server system 140 can transmit the weights of the trained model(s) to therapy management engine 114, which could execute on display device 107, etc. The model(s) can then be used to predict, in real-time, an optimal dosage, an expected weight loss of a patient, gastrointestinal symptoms, and/or any of the other predictions discussed herein using application 106, and/or make other types of recommendations discussed above. In certain embodiments, the training server system 140 can continue to train the model(s) in an “online” manner by using input features and labels associated with new patient records.

Further, similar methods for the training illustrated in FIG. 7, historical patient records can also be used to train models using patient-specific records to create more personalized models. For example, a model trained using historical patient records that is deployed for a particular patient, can be further re-trained after deployment. For example, the model can be re-trained after the model is deployed for a specific patient to create a more personalized model for the patient. The more personalized model can be able to more accurately make predictions on the optimal GLP-1 regimen based on weight loss of the patient and the presence of gastrointestinal symptoms, and provide recommendations for medication parameters, specific diet recommendations, meal times, and/or exercise regimens to minimize symptoms, optimize weight loss, and/or maintain weight loss based on the patient's own data (as opposed to only historical patient record data), including the patient's own inputs 130 and metrics 132.

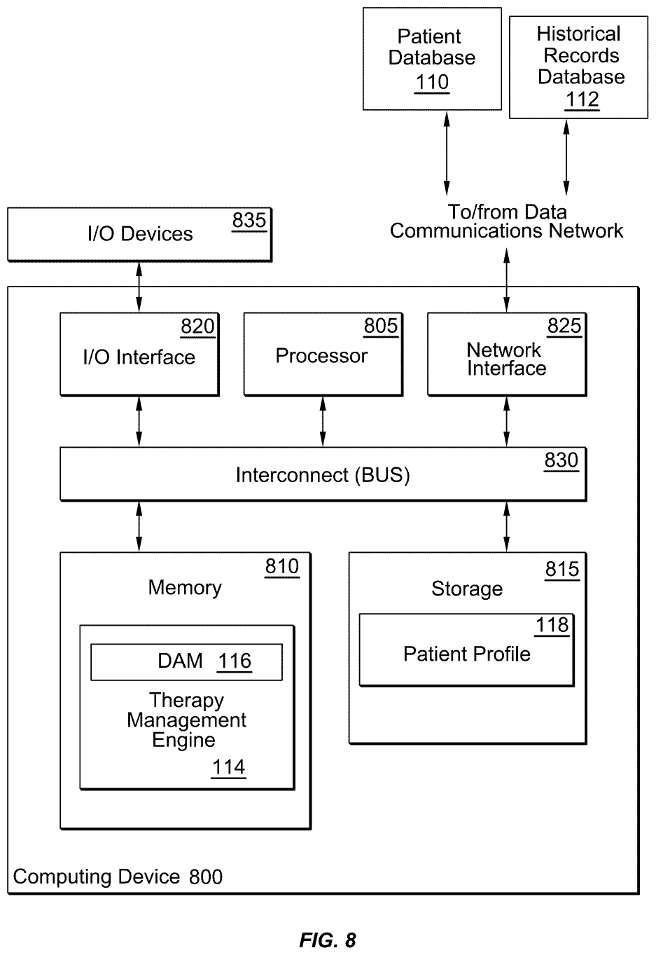

FIG. 8 is a block diagram depicting a computing device 800 configured to execute a therapy management engine (e.g., therapy management engine 114), according to certain embodiments disclosed herein. Although depicted as a single physical device, in embodiments, computing device 800 can be implemented using virtual device(s), and/or across a number of devices, such as in a cloud environment. As illustrated, computing device 800 includes a processor 805, memory 810, storage 815, a network interface 825, and one or more I/O interfaces 820. In the illustrated embodiment, processor 805 retrieves and executes programming instructions stored in memory 810, as well as stores and retrieves application data residing in storage 815. Processor 805 is generally representative of a single CPU and/or GPU, multiple CPUs and/or GPUs, a single CPU and/or GPU having multiple processing cores, and the like. Memory 810 is generally included to be representative of a random-access memory. Storage 815 can be any combination of disk drives, flash-based storage devices, and the like, and can include fixed and/or removable storage devices, such as fixed disk drives, removable memory cards, caches, optical storage, network attached storage (NAS), or storage area networks (SAN).

In some embodiments, input and output (I/O) devices 835 (such as keyboards, monitors, etc.) can be connected via the I/O interface(s) 820. Further, via network interface 825, computing device 800 can be communicatively coupled with one or more other devices and components, such as patient database 110. In certain embodiments, computing device 800 is communicatively coupled with other devices via a network, which can include the Internet, local network(s), and the like. The network can include wired connections, wireless connections, or a combination of wired and wireless connections. As illustrated, processor 805, memory 810, storage 815, network interface(s) 825, and I/O interface(s) 820 are communicatively coupled by one or more interconnects 830. In certain embodiments, computing device 800 is representative of display device 107 associated with the patient. In certain embodiments, as discussed above, the display device 107 can include the patient's laptop, computer, smartphone, and the like. In another embodiment, computing device 800 is a server executing in a cloud environment.

In the illustrated embodiment, storage 815 includes patient profile 118. Memory 810 includes therapy management engine 114, which itself includes DAM 116.

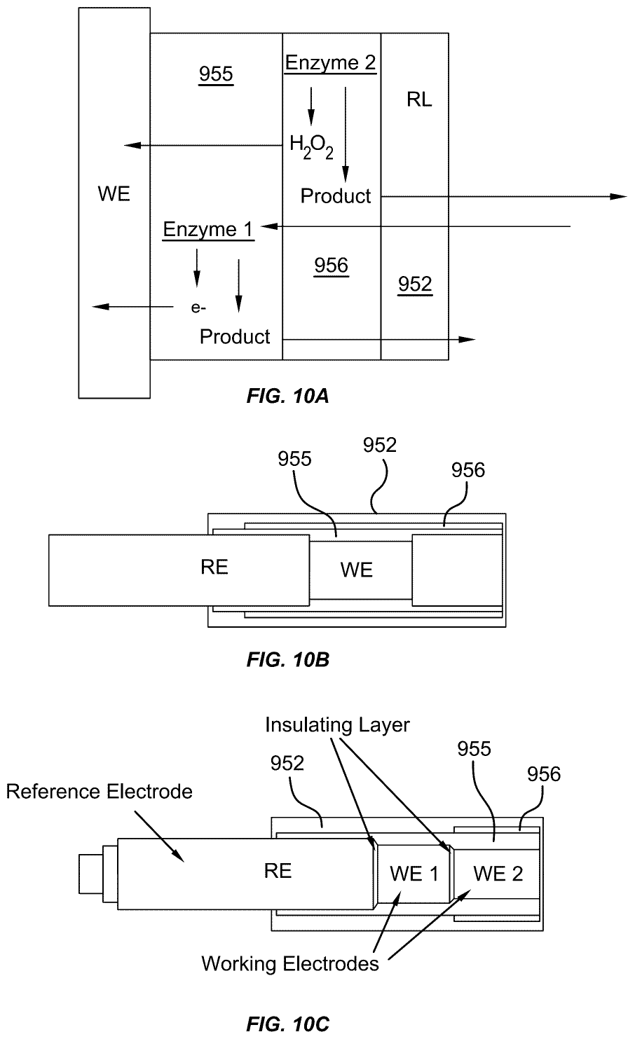

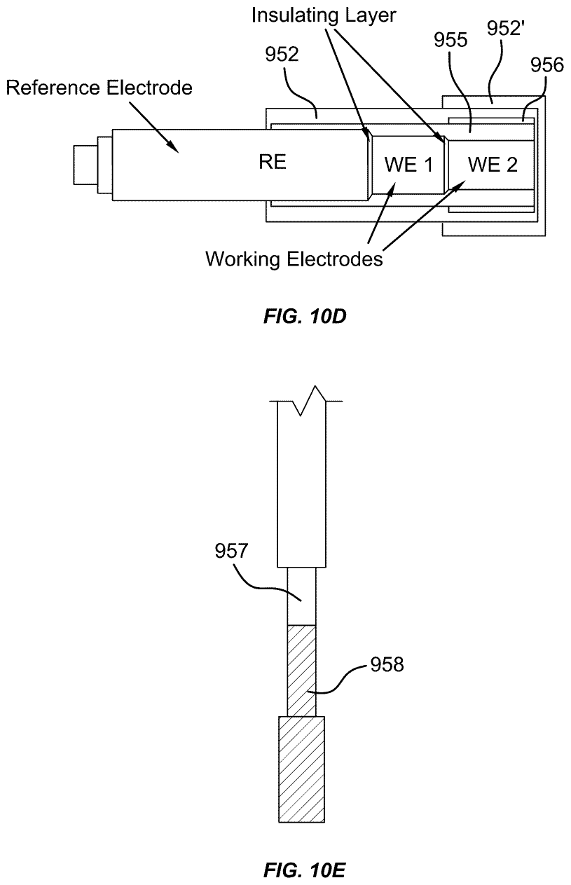

As described above, continuous analyte monitoring system 104, described in relation to FIG. 1, can be a multi-analyte sensor system including a multi-analyte sensor. FIGS. 9-10 describe example multi-analyte sensors used to measure multiple analytes.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, an ionophore, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, lactate, ketone, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection can be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include adjustment or measurement of current and/or voltage, inductance, capacitance, impedance, transconductance, and charge. Optical properties include absorbance, fluorescence/phosphorescence, fluorescence/phosphorescence decay rate, wavelength shift, dual wave phase modulation, bio/chemiluminescence, reflectance, light scattering, Raman shift, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more single or double stranded DNA or RNA moieties. Transcutaneous continuous multi-analyte sensors can be used in vivo over various lengths of time. The continuous multi-analyte sensor systems discussed herein can be transcutaneous devices, in that a portion of the device can be inserted through the host's skin and into the underlying soft tissue while a portion of the device remains on the surface of the host's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the host's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels or lactate levels. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, electrodeposition, plasma polymerization, electrospraying), or thick film techniques (spray coating, dip coating, spin coating, pad printing, screen printing, discrete dispense, inkjet deposition, slot-die coating, brush coating, film coating, drop-let coating, and the like). Additional steps can be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (m), or less, to about 1, 2, 3, 4, 5, 6, 7, 8, 9, 10, 11, 12, 13, 14, 15, or 16 m is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether can be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering can be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as electrodeposition, plasma polymerization, electrospraying, spray coating, dip coating, casting, electrospinning, vapor deposition, spin coating, pad printing, screen printing, discrete dispense, inkjet deposition, solt-die coating, brush coating, film coating, droplet coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure or otherwise cross-link by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties, including the hydrophilic and hydrophobic domains dispersed throughout the film. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spray coating, self-assembling monolayers (SAMs), painting, dip coating, vapor depositing, electrodepositing, plasma polymerizing, electrospraying, pad printing, spin coating, discrete dispensing, inkjet depositing, slot-die coating, molding, 3-D printing, lithographic techniques (e.g., photolithograph), micro- and nano-pipetting printing techniques, screen printing, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor can promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein can include a biointerface layer. The biointerface layer, like the drug releasing layer, can include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein can include a drug releasing membrane at least partially functioning as or in combination with a biointerface membrane. The drug releasing membrane can include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Embodiments of the present disclosure advantageously provide continuous multi-analyte sensors with various membrane configurations suitable for facilitating signal transduction corresponding to analyte concentrations, either simultaneously, intermittently, and/or sequentially. In one example, such sensors can be configured using a signal transducer, comprising one or more transducing elements. Such continuous multi-analyte sensors can employ various transducing means and methods, such as amperometric, voltametric, chronoamperometric, coulometric, chronocoulometric, potentiometric, conductance, and impedimetric methods, among other techniques.

In one example, the transducing element comprises one or more membranes that can comprise one or more layers and or domains, each of the one or more layers or domains can independently comprise one or more signal transducers, e.g., enzymes, ionophores, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain can comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte/oxidase enzyme pairs: for example, glucose/glucose oxidase, lactate/lactate oxidase, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, L-amino acid oxidase, catechol/catechol oxidase, beta-hydroxybutyrate/beta-hydroxybutyrate oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, enzyme cascades, dimerized and/or fusion enzymes.

### NAD Based Multi-Analyte Sensor Platform

Nicotinamide adenine dinucleotide (NAD(P)⁺/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)⁺) and reduced form (NAD(P)H) (H=hydrogen). The reaction of NAD+ and NADH is reversible, thus, the coenzyme can continuously cycle between the NAD(P)⁺/and NAD(P)H forms essentially without being consumed. NAD(P)⁺ and NAD(P)H are electroactive and can undergo reduction and oxidation reactions, respectively, at a suitable bias potential.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, the redox-active cofactors NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+reduction into measurable current with amperometry. In one example, described below, continuous, sensing of multi-analytes, either reversibly bound or at least one of which are oxidized or reduced by NAD+ dependent enzymes, for example, glucose (glucose dehydrogenase), lactate (lactate dehydrogenase), glycerol (glycerol dehydrogenase), cortisol (11β-hydroxysteroid dehydrogenase), alcohol (alcohol dehydrogenase), aldehydes (aldehyde dehydrogenase), and ketones (beta-hydroxybutyrate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 9A. With reference to FIG. 9B, one or more optional layers can be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 9A-9B, one or more of the membranes provides a NAD+ reservoir domain providing a reservoir for NAD+. In one example, one or more interferent blocking membranes is used, and potentiostat is utilized to measure H2O2 production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, methylene blue, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

Another example of a continuous ketone analyte detection configuration employing electrode-associated mediator-coupled diaphorase/NAD+/dehydrogenase is depicted below:

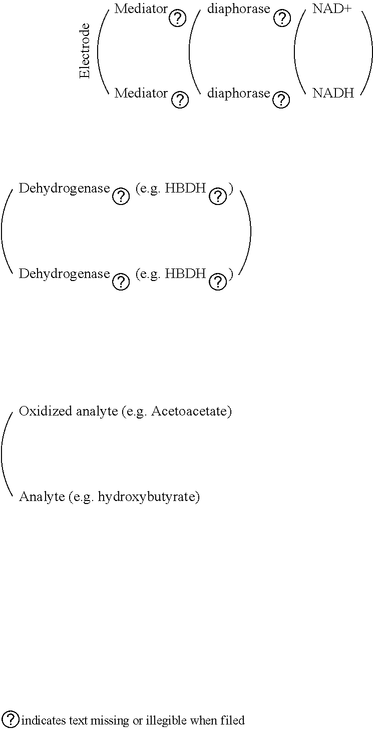

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ can be covalently coupled to an aspect of the enzyme domain having a higher molecular weight than the NAD+ which can improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a single analyte sensor configuration suitable for combination with another analyte sensor configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE(400MW). The substrates discussed herein that can include working electrodes can be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous analyte sensor as depicted in FIGS. 9A-9B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) can also contain a polymer or protein binder, such as zwitterionic polyurethane, and/or albumin. Alternatively, in addition to NAD(P)H, the membrane can contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer(or protein), and can be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous analyte sensor configurations can be adapted to other analytes or used in combination with other sensor configurations. For example, analyte(s)-dehydrogenase enzyme combinations can be used in any of the membranes of the sensing region include; glucose (glucose dehydrogenase); lactate (lactate dehydrogenase); glycerol (glycerol dehydrogenase); cortisol (11β-hydroxysteroid dehydrogenase); alcohol (alcohol dehydrogenase); and aldehydes (aldehyde dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. FIG. 9C depicts this exemplary configuration, of an enzyme domain 950 comprising an enzyme (Enzyme) with an amount of cofactor (Cofactor) that is positioned proximal to at least a portion of a working electrode (“WE”) surface, where the WE comprises an electrochemically reactive surface. In one example, a second membrane 951 comprising an amount of cofactor is positioned adjacent the first enzyme domain. The amount of cofactor in the second membrane can provide an excess for the enzyme, e.g., to extend sensor life. One or more resistance domains 952 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 9D depicts an alternative enzyme domain configuration comprising a first membrane 951 with an amount of cofactor that is positioned more proximal to at least a portion of a WE surface. Enzyme domain 950 comprising an amount of enzyme is positioned adjacent the first membrane.

In the membrane configurations depicted in FIGS. 9C-9D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

### Glucose Sensor Configurations

In certain embodiments, continuous glucose sensor device configurations are provided. In one example, glucose oxidase (GOx) can be included in one or more enzyme domains and positioned adjacent the working electrode surface. The catalysis of the glucose using GOx, in the presence of dissolved oxygen, produces hydrogen peroxide which can be detected and/or measured qualitatively or quantitatively, using amperometric, voltametric, chronoamperometric, coulometric, chronocoulometric, potentiometric, conductance, and impedimetric methods.

In certain embodiments, the sensing region for the enzyme substrate-oxidase enzyme configurations has one or more enzyme domains which include one or more electrodes. In certain embodiments, the sensing region has one or more enzyme domains, with or without one or more electrodes, and one or more interference blocking membranes (e.g., permselective membranes, and/or charge exclusion membranes). The one or more interference blocking membranes can attenuate one or more interferents from diffusing through the membrane to the working electrode. In certain embodiments, the sensing region has one or more enzyme domains, with or without the one or more electrodes, and one or more resistance domains, with or without the one or more interference blocking membranes. In certain embodiments, the sensing region has one or more enzyme domains, with or without the one or more electrodes, one or more resistance domains, with or without the one or more interference blocking membranes, and one or more biointerface membranes and/or drug releasing membranes. The one or more biointerface membranes and/or drug releasing membranes attenuate the diffusion of one or more analytes or enzyme substrates and attenuate the immune response of the host after insertion.

In one example, the one or more interference blocking membranes are deposited on a surface anterior to the working electrode and/or the electrode surface. In one example, the one or more interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing through the sensing region. Such membranes can be used to attenuate glucose itself as well as attenuate other electrochemically actives species or other analytes that can otherwise interfere by producing a signal if they diffuse to the working electrode. Said membranes can be engineered to allow glucose permeation to occur relatively unabated. Said permselectivity properties can be based on molecular weight.

In one example, the working electrode used comprised platinum and the potential applied is about 0.6 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of glucose. When appropriately designed to obey stoichiometric behavior, the presence of a specific concentration of glucose should cause a commensurate reduction in local oxygen in a direct (linear) relation with the concentration of glucose. Accordingly, a multi-analyte sensor for both glucose and oxygen can therefore be provided.

In another example, the above mentioned glucose sensor configuration can include one or more mediators. In one example, the one or more mediators are present in, on, or about one or more electrodes or electrode surfaces and/or are deposited or otherwise associated with the surface of the WE or reference electrode (“RE”). In one example, the one or more mediators eliminate or reduce direct oxidation of interfering species that can reach the WE or RE. In one example, the one or more mediators provide a lowering of the operating potential of the WE/RE, for example, from about 0.6V to about 0.3V or less on a platinum electrode, which can reduce or eliminate oxidation of endogenous interfering species. Examples of one or mediators are provided above, such as methylene blue, polyvinylimidizole-Os(bpy)₂Cl, etc. Other electrodes, such as counter electrodes, can be employed.

In one example, other enzymes or additional components can be added to the polymer mixture(s) that constitute any part of the sensing region to increase the stability of the aforementioned sensor and/or reduce or eliminate the byproducts of the glucose/glucose oxidase reaction. In another example, the additional components can also be proteins (e.g., bovine serum albumin (BSA)) or cross-linkers (e.g., glutaraldehyde, N,N′-Carbonyldiimidazole (CDI)). Increasing stability includes storage or shelf life and/or operational stability (such as retention of enzyme activity during use). For example, byproducts of enzyme reactions can be undesirable for increased shelf life and/or operational stability, and can thus be desirable to reduce or remove. In one example, xanthine oxidase can be used to remove byproducts of one or more enzyme reactions.

In another example, a dehydrogenase enzyme is used with an oxidase for the detection of glucose alone or in combination with oxygen. Thus, in one example, glucose dehydrogenase is used to oxidize glucose to Glucono-δ-lactone in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled glucose dehydrogenase (GDH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ can be coupled to a polymer, such as dextran, and the polymer immobilized in the enzyme domain along with GDH. This provides for retention of the co-factor and availability thereof for the active site of GDH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface can be provided. A chemical moiety capable of assisting with electron transfer from the enzyme or cofactor to the electrode surface can be used and includes one or more mediators as described below.

In one example, any one of the aforementioned continuous glucose sensor configurations are combined with any one of the aforementioned continuous analyte monitoring configurations to provide a continuous multi-analyte sensor device as further described below. In one example, a continuous glucose monitoring configuration combined with any one or more of the aforementioned continuous analyte sensor configurations to provide a continuous multi-analyte sensor device as further described below.

### Lactate Sensor Configurations

In another example, a continuous lactate sensor device configuration is provided. Thus, in one example, lactate oxidase (LOx) can be included in one or more enzyme domains and positioned adjacent the working electrode surface. In the presence of oxygen, the catalysis of the lactate using LOx, produces hydrogen peroxide which can be detected and/or measured qualitatively or quantitatively, using, among other techniques, amperometry, voltametric, chronoamperometric, coulometric, chronocoulometric, potentiometric, conductance, and impedimetric methods.

In certain embodiments, the sensing region for the enzyme substrate-oxidase enzyme configurations has one or more enzyme domains which include one or more electrodes. In certain embodiments, the sensing region has one or more enzyme domains, with or without one or more electrodes, and one or more interference blocking membranes (e.g., permselective membranes, and/or charge exclusion membranes). The one or more interference blocking membranes can attenuate one or more interferents from diffusing through the membrane to the working electrode. In certain embodiments, the sensing region has one or more enzyme domains, with or without the one or more electrodes, and one or more resistance domains, with or without the one or more interference blocking membranes. In certain embodiments, the sensing region has one or more enzyme domains, with or without the one or more electrodes, one or more resistance domains, with or without the one or more interference blocking membranes, and one or more biointerface membranes and/or drug releasing membranes. The one or more biointerface membranes and/or drug releasing membranes attenuate one or more analytes or enzyme substrates and attenuate the immune response of the host after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate lactate itself as well as attenuate other electrochemically actives species or other analytes that can otherwise interfere by producing a signal if they diffuse to the working electrode.

In one example, the working electrode used comprised platinum and the potential applied is about 0.6 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of lactate. When appropriately designed to obey stoichiometric behavior, the presence of a specific concentration of lactate should cause a commensurate reduction in local oxygen in a direct (linear) relation with the concentration of lactate. Accordingly, a multi-analyte sensor for both lactate and oxygen can therefore be provided.

In another example, the above-mentioned lactate sensor configuration can include one or more mediators. In one example, the one or more mediators are present in, on, or about one or more electrodes or electrode surfaces and/or are deposited or otherwise associated with the surface of the WE or RE. In one example, the one or more mediators eliminate or reduce direct oxidation of interfering species that can reach the WE or RE. In one example, the one or more mediators provide a lowering of the operating potential of the WE/RE, for example, from about 0.6V to about 0.3V or less on a platinum, gold, or carbon electrode, which can reduce or eliminates oxidation of endogenous interfering species. Examples of suitable mediators are provided below. Other electrodes, e.g., counter electrodes, can be employed.

In one example, other enzymes or additional components can be added to the polymer mixture(s) that constitute any part of the sensing region to increase the stability of the aforementioned sensor and/or reduce or eliminate the byproducts of the lactate/lactate oxidase reaction. In certain embodiments, the enzymes or additional components that can be added to increase stability can be cross-linked polymers and/or enzyme stabilizers. Increasing stability includes storage or shelf life and/or operational stability (e.g., retention of enzyme activity during use). For example, byproducts of enzyme reactions can be undesirable for increased shelf life and/or operational stability, and can thus be desirable to reduce or remove.

In another example, a dehydrogenase enzyme is used with an oxidase for the detection of lactate alone or in combination with oxygen. Thus, in one example, lactate dehydrogenase is used to oxidize lactate to pyruvate in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor. Said NAD(P)H, in certain configurations, can be oxidized directly with the application of a suitable bias potential.

In the aforementioned dual enzyme configuration, a signal can be sensed either by: (1) an electrically coupled lactate dehydrogenase, for example, using an electro-active hydrogel polymer comprising one or more mediators; (2) oxygen electrochemical sensing to measure the oxygen consumption of the NADH oxidase; or (3) hydrogen peroxide electrochemical sensing to measure the hydrogen peroxide generation by means of NADH oxidase in the presence of NADH and oxygen. In an alternative example, the co-factor NAD(P)H or NAD(P)+ can be coupled to a polymer, such as dextran, the polymer immobilized in the enzyme domain along with PDH. This provides for retention of the co-factor and availability thereof for the active site of PDH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface can be provided. A chemical moiety capable of assisting with electron transfer from the enzyme or cofactor to the electrode surface can be used and includes one or more mediators as described below.

In one example, any one of the aforementioned continuous lactate sensor configurations are combined with any one of the aforementioned continuous analyte monitoring configurations to provide a continuous multi-analyte sensor device as further described below. In one example a continuous lactate monitoring configuration combined with the aforementioned continuous glucose sensor configuration to provide a continuous multi-analyte sensor device as further described below.

### One-Working-Electrode Configurations for Dual Analyte Detection

In one example, at least a dual enzyme domain configuration in which each layer contains one or more specific enzymes and optionally one or more cofactors is provided. In a broad sense, one example of a continuous multi-analyte sensor configuration is depicted in FIG. 10A where a first membrane 955 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 956 (EZL2) with at least one second enzyme (Enzyme 2) is positioned adjacent 955 ELZ1, and is generally more distal from WE than EZL1. One or more resistance domains (RL) 952 can be provided adjacent EZL2 956, and/or between EZL1 955 and EZL2 956. The different enzymes catalyze the transformation of the same analyte, but at least one enzyme in EZL2 956 provides hydrogen peroxide and the other at least one enzyme in EZL1 955 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 10A, a first analyte diffuses through RL 952 and into EZL2 956 resulting in generation of hydrogen peroxide via interaction with Enzyme 2. Hydrogen peroxide diffuses at least through EZL1 955 to WE and transduces a signal that corresponds directly or indirectly to the first analyte concentration. A second analyte, which is different from the first analyte, diffuses through RL 952 and EZL2 956 and interacts with Enzyme 1, which results in electron transfer to WE and transduces a signal that corresponds directly or indirectly to the second analyte concentration.

As shown in FIG. 10B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 956 providing hydrogen peroxide and the at least other enzyme in EZL1 955 not providing hydrogen peroxide, e.g., providing electron transfer to the WE surface corresponding directly or indirectly to a concentration of the analyte.

In one example, an inner layer of the at least two enzyme domains EZL1, EZL2 955, 956 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 955 is more proximal to the WE surface and can have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 955 is directly adjacent the WE.

The second layer of at least dual enzyme domain (the outer layer EZL2 956) of FIG. 10B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 956 and through the inner layer EZL1 955 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry, pulsed amperometric detection, linear sweep voltammetry, differential pulse voltammetry, square wave voltammetry, etc. In some examples, impedimetric sensing can be used. In one example, a phase shift (e.g., a time lag) can result from detecting two signals from two different working electrodes, each signal being generated by a different EZL (EZL1, EZL2, 955, 956) associated with each electrode. The two (or more) signals can be broken down into components to detect the individual signal and signal artifacts generated by each of EZL1 955 and EZL2 956 in response to the detection of two analytes. In some examples, each EZL detects a different analyte. In other examples, both EZLs detect the same analyte.

In another alternative exemplary configuration, as shown in FIGS. 10C-10D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate.

Thus, FIGS. 10C-10D depict exemplary configurations of a continuous multi-analyte sensor construct in which EZL1 955, EZL2 956 and RL 952 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 10C-10D, WE1 represents a first working electrode surface configured to operate at P1, WE2 represents a second working electrode surface configured to operate at P2, WE1 is electrically insulated from WE2, and RE represents a reference electrode electrically isolated from both WE1, WE2. One resistance domain is provided in the configuration of FIG. 10C that covers the RE and WE1, WE2. An additional resistance domain is provided in the configuration of FIG. 10D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (hydrogen peroxide producing producing) and glucose dehydrogenase (electrically coupled) configuration. Measurement of glucose at two potentials and from two different electrodes provides more data points and accuracy. Such approaches can not be needed for glucose sensing, but can be applied across the biomarker sensing spectrum of other analytes, alone or in combination with glucoses sensing, such as lactate sensing and glucose/lactate sensing.

In an alternative configuration of that depicted in FIGS. 10C-10D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 10D, such an arrangement of RL's is depicted, where an additional RL 952′ is adjacent WES2 but substantially absent from WES1.

In one example of measuring two different analytes, the above configuration comprising enzyme domain EZL1 955 comprising one or more enzyme(s) and one or more mediators for at least one enzyme of EZL1 to provide for direct electron transfer to the WES1 and determining a concentration of at least a first analyte. In addition, enzyme domain EZL2 956 can comprise at least one enzyme that provides peroxide (e.g., hydrogen peroxide) or consumes oxygen during catalysis with its substrate. The peroxide or the oxygen produced in EZL2 956 migrates to WES2 and provides a detectable signal that corresponds directly or indirectly to a second analyte. For example, ELZ1 955 can be lactate oxidase, ELZ2 956 can be glucose oxidase, and WES2 can be carbon, wired to glucose oxidase to measure glucose, while WES1 can be platinum, that measures peroxide produced from lactate oxidase/lactate in EZL1 955. The combinations of electrode material and enzyme(s) as disclosed herein are examples and non-limiting.

In one example, the potentials of P1 and P2 can be separated by an amount of potential so that both signals (from direct electron transfer from EZL1 955 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., pulsed amperometric detection. In one example, an alternative configuration is provided but hydrogen peroxide production in EZL2 is replaced by another suitable electrolysis compound that maintains the P2 #P1 relationship, such as oxygen, and at least one enzyme-substrate combination that provide the other electrolysis compound.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 10E, an example of such composite electrode surfaces is shown, in which an extended platinum covered wire 957 is half coated with carbon 958, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that can be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include lactate sensing (lactate dehydrogenase electrically coupled enzyme in EZL1 955) and glucose sensing (glucose oxidase in EZL2 956). Other membranes can be used in the aforementioned configuration, such as electrode, resistance, bio-interfacing, and drug releasing membranes. In other examples, one or both of the working electrodes (WE1, WE2) can be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein can instead or additionally include graphene, graphene oxide, or other materials suitable for forming the working electrodes, such as commercially available carbon ink.

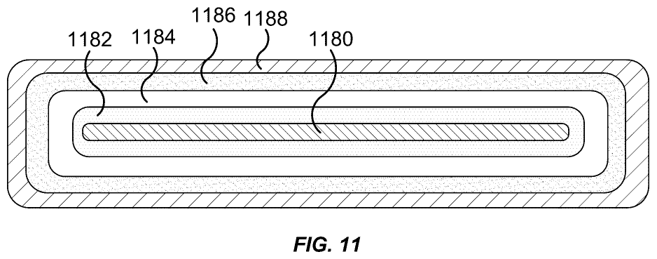

FIG. 11 is an illustration of an example planar analyte sensor with sensing membranes, according to certain embodiments of the present disclosure. The planar analyte sensor can include electrode 1180 with a sensing membrane with multiple layers or domains. For example, the planar version can include an interference domain 1182, an enzyme domain 1184, and resistance domain 1186, in addition to other variations of domains, such as drug releasing membrane 1188. As shown, the planar analyte sensor includes sensing membrane surrounding the electrically conductive material or electrode 1180, however, the electrically conductive material or electrode 1180 can be on one side thereof in other examples.

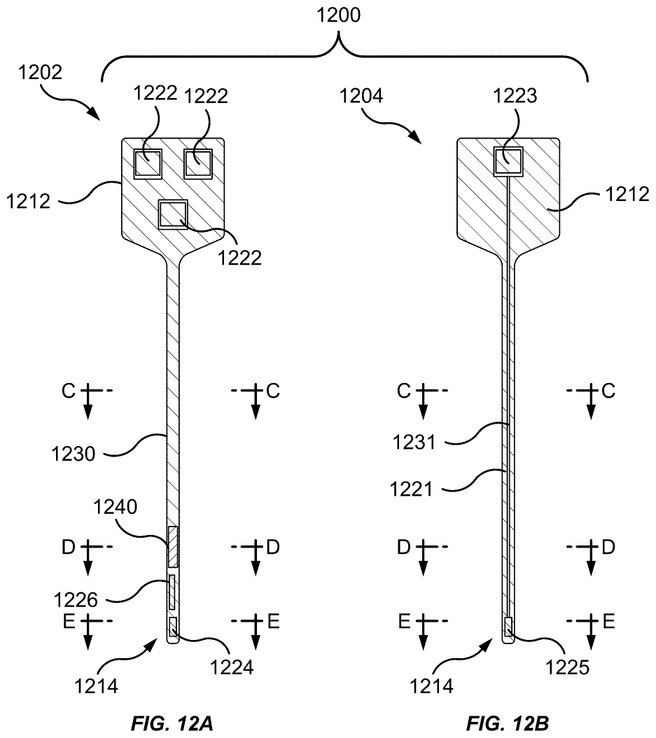

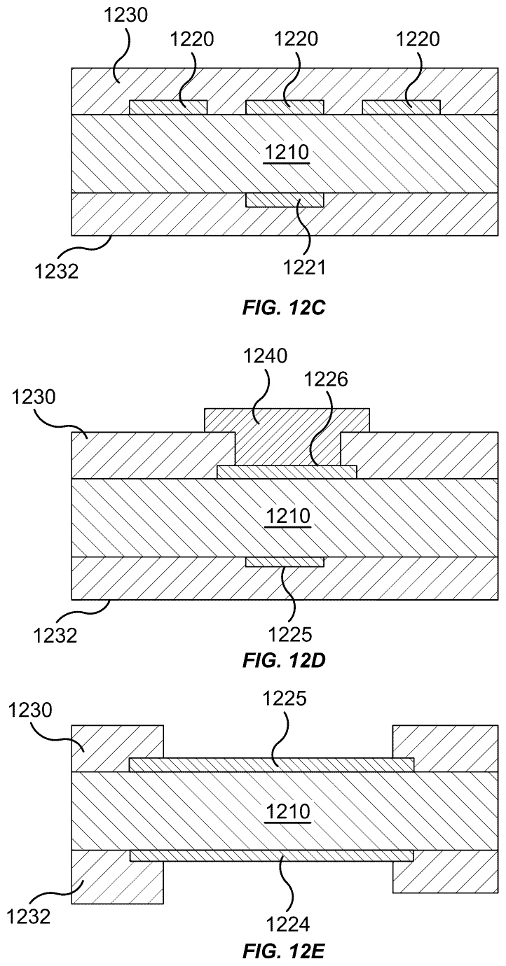

FIGS. 12A to 12B depict an exemplary planar sensor assembly 1200, showing top-down drawings of a first side 1202 and a second side 1204 opposite the first side, in addition to a first end 1212 and a second end 1214. FIGS. 12C to 12E depict schematic cross-section drawings of the full sensor assembly 1200. The sensor assembly 1200 can include substrate 1210, conductive traces 1220, 1221, connector pads 1222, 1223, WEs 1224, 1225, counter electrode 1226, insulating layers 1230, 1232, and reference electrode 1240. In sensor assembly 1200, a double-sided planar configuration is used. In the sensor assembly 1200, a multiple-electrode sensor is shown, with two WEs 1224, 1225, a counter electrode (CE) 1226 and a reference electrode (RE) 1240. In sensor assembly 1200, the electrodes are co-planar. The sensor assembly 1200 is an unconnected variation. In examples, WEs 1224, 1225 are coated with the sensing membrane with multiple layers or domains as disclosed herein.

In sensor assembly 1200, structures can be formed on both sides 1202, 1204, of the substrate 1210. For example, the connector pads 1222, 1223, can be formed, respectively, on opposing sides 1202, 1204. This can allow for connection to the sensing electronics from both sides of the sensor assembly 1200. Similarly, the conductive traces 1220, 1221, can be formed on both sides 1202, 1204, of the sensor assembly 1200. On each individual side 1202, 1204 the conductive traces 1220, 1221, can be co-planar with each other.

The insulating layers 1230, 1232, such as a solder mask or other insulating material, can be deposited over the conductive layers including the conductive traces 1220, 1221. Openings can be formed in the insulating layers 1230, 1232, to form the WEs 1224, 1225, and the CE 1226. An opening can be left for the RE 1240. A RE material, such as silver/silver chloride, can be deposited on the designated sensing surface for the RE 1240. The insulating material can include epoxy, polyimide, polyurethane, polyethylene, or other materials or combinations of materials.

As illustrated in FIGS. 12A and 12B, the double-sided sensor assembly 1200 can include a first WE 1224, a second WE 1225, a CE 1226, and a RE 1240. In some cases, such a double-sided sensor can contain more or less electrodes. For example, a double-sided sensor can include a single WE and a RE or two WEs and a single RE.

FIGS. 12C to 12E depict cross-sections of the sensor assembly 1200. Shown in FIG. 12C is a cross section along line C-C, where the substrate 1210 is situated between the two insulating layers 1230, 1232. The substrate 1210 can be, for example, about 50 microns thick. Conductive traces 1220, 1221, can be seen. On the first side 1202, three conductive traces 1220 extend along the length of the sensor assembly 1200, each connecting to a connector pad 1222. The conductive traces 1221 on the second side 1204 can connect to the connector pad 1223.

In FIG. 12D, the cross-section is taken along line D-D. The RE 1240 can be seen at this point. In FIG. 12E, the cross-section is taken along line E-E, both WEs 1224, 1225, can be seen on opposing sides 1202, 1204, of the sensor assembly 1200.

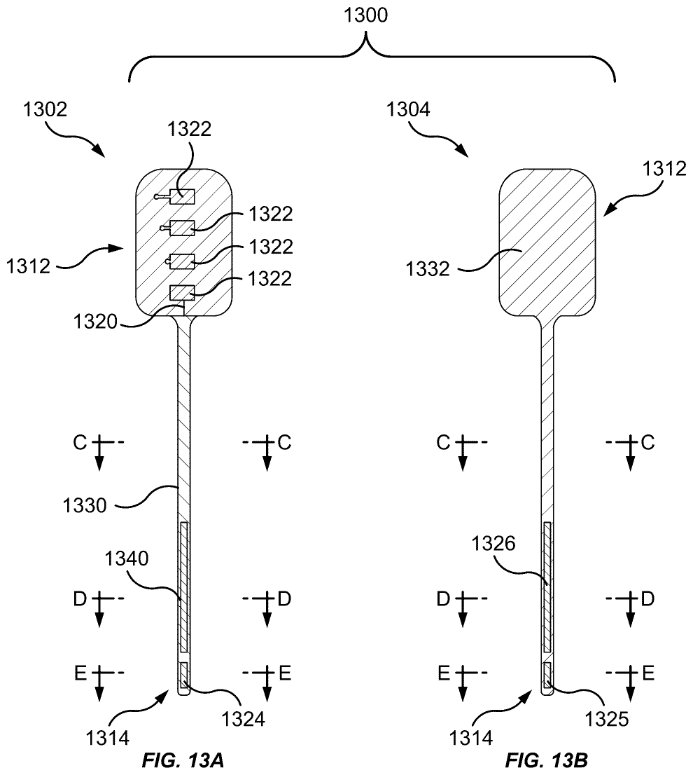

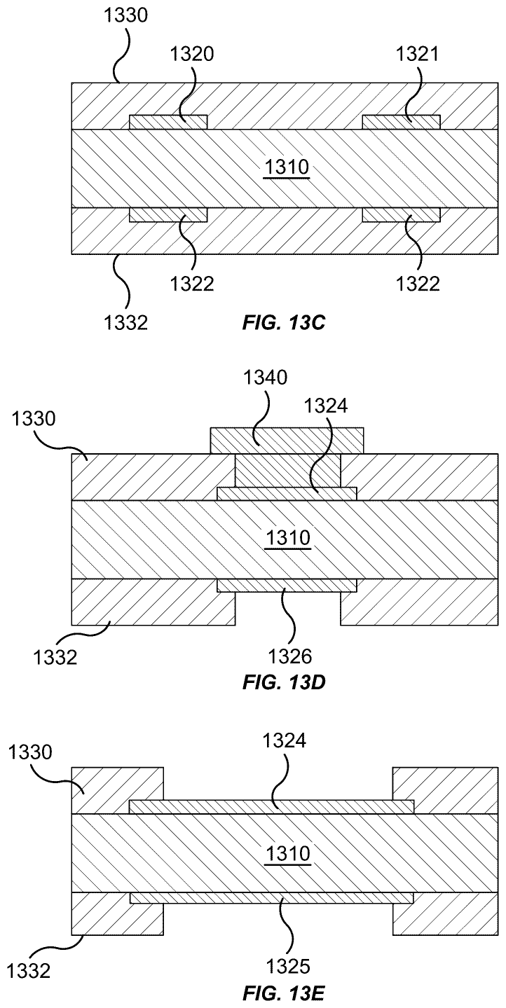

FIGS. 13A-13B illustrate a double-sided co-planar connected analyte sensor assembly 1300, in accordance with an example. The sensor assembly 1300 can include similar components to those of assembly 1200 discussed above, except where otherwise noted.

FIGS. 13A to 13B depict schematic top-down drawings of opposing sides of the assembly 1300. FIGS. 13C to 13E depict schematic cross-section drawings along cross-sections taken along C-C, D-D, and E-E, respectively, of the full sensor assembly 1300. In some cases, the sensor assembly 1300 can include a chamfer end, a rounded end, a flat end, or other appropriate shape. In some cases, the sensor assembly 1300 can include a chamfer end, a rounded end, a flat end, or other appropriate shape.

The sensor assembly 1300 can have a first side 1302 and a second side 1304 opposite the first side, in addition to a first end 1312 and a second end 1314. The sensor assembly 1300 can include substrate 1310, conductive traces 1320, 1321, connector pads 1322, WEs 1324, 1325, CE 1326, insulating layers 1330, 1332, and RE 1340. In sensor assembly 1300, a double-sided planar configuration is used. In the sensor assembly 1300, a multiple-electrode sensor is shown, with two WEs 1324, 1325, a CE 1326 and RE 1340. In sensor assembly 1300, the electrodes are co-planar. The assembly 1300 is a co-planar, connected variation.

In sensor assembly 1300, the substrate 1310 is situated between two sides 1302, 1304, which can each host several co-planar components. For example, co-planar conductive traces 1320 can be on the first side 1302, and second conductive traces 1321 can be on the second side 1304. Each side 1302, 1304, can be covered by an insulating layer 1330, 1332. The insulating layers 1330, 1332, can define electrodes 1324, 1325, 1326, and an area for the RE 1340.

The assembly 1300 can also include a via, which can provide for an electrical connection between both sides 1302, 1304 of the sensor assembly 1300. A via can also be used within the substrate to connect buried conductive traces occupying differing layers of the assembly. Including vias can allow for connection to the sensing electronics through the connector pads 1322 on a single side 1302 of the sensor, as well as routing traces to new locations, allowing flexible geometries to be used. The vias can be formed from various conductive materials discussed herein, including gold, carbon, graphitic carbon, Pt, Pd, Ni, Cu, or combinations including Pt and C, Au and C. In some examples, the conductive material forming the vias between sides 1302, 1304 of the assembly or other assemblies as discussed herein may or may not further include conductive nanoparticles.

Shown in FIGS. 13A to 13E, the assembly 1300 can include four connector pads 1322 can be on a first side 1302, electrically coupled to the electrodes 1325, 1340, on the second side 1304 by vias and traces. In some cases, a WE, RE, and CE can be placed on the opposite side of the sensor assembly 1300 to the connector pads 1322. In some cases, as shown in assembly 1300, a first WE 1324 and CE 1326 can be located on the first side 1302 of the sensor, while a second WE 1325 and a RE 1340 can be located on the other side 1304 of the sensor. Vias can be used to establish electrical contact between traces and pads on both sides 1302, 1304 of the sensor assembly 1300, since the connector pads 1322 for connecting to the sensing electronics, in some examples, are located only on one side. In examples, WEs 1324, 1325 are coated with the sensing membrane with multiple layers or domains as disclosed herein.

### Electrodes

Examples of electrodes suitable for use in the devices and methods disclosed herein include, for example, platinum and its binary and tertiary alloys, palladium and its binary and tertiary alloys, gold and its binary and tertiary alloys, silver and its binary and tertiary alloys, iridium or indium and its binary and tertiary alloys, rhodium, ruthenium, nitinol, indium tin oxide, bismuth molybdate (Bi2MoO6), tin sulfide metal oxide (SnS2), boron doped diamond, platinum coated boron doped diamond, conductive graphite and inks therefrom, gold, platinum, pallidum or iridium coated silicon wafers, doped polyaniline, doped poly(3,4-ethylenedioxythio-phene) polystyrene sulfonate (PEDOT:PSS), doped polypyrrole (Ppy), amorphous carbon, carbon nanotubes, graphene metallic nanoparticles, and/or ternary metal oxide composites. The electrode may be roughened, via electrochemical or other physical or chemical etching means. Roughening the electrode augments the electroactive surface area available for a reaction of interest to occur, thereby augmenting detected signal level.

### Sensor/Sensor System

Exemplary sensors are described previously herein. In some examples, the core and first layer can be of a single material (e.g., platinum). In some examples, the elongated conductive body is a composite of at least two materials, such as a composite of two conductive materials, or a composite of at least one conductive material and at least one non-conductive material. In some examples, the elongated conductive body comprises a plurality of layers. In certain examples, there are at least two concentric (e.g., annular) layers, such as a core formed of a first material and a first layer formed of a second material. However, additional layers can be included in some examples. In some examples, the layers are coaxial.

The elongated conductive body may be long and thin, yet flexible and strong. For example, in some examples, the smallest dimension of the elongated conductive body is less than about 0.1 inches, 0.75 inches, 0.5 inches, 0.25 inches, 0.01 inches, 0.004 inches, or 0.002 inches. While the elongated conductive body is shown as having a circular or substantially circular cross-section in some examples, in other examples the cross-section of the elongated conductive body is ovoid, rectangular, triangular, polyhedral, star-shaped, C-shaped, T-shaped, X-shaped, Y-Shaped, irregular, or the like. In examples, a conductive wire electrode is employed as a core. To such a clad electrode, two additional conducting layers may be added (e.g., with intervening insulating layers provided for electrical isolation). The conductive layers can be comprised of any suitable material. In certain examples, it can be desirable to employ a conductive layer comprising conductive particles (i.e., particles of a conductive material) in a polymer or other binder. In other examples, the conductive body can be configured in a linear or planar arrangement, e.g., on a generally flat surface or substrate.

In addition to providing structural support, resiliency and flexibility, in some examples, the core (or a component thereof) provides electrical conduction for an electrical signal from the working electrode to sensor electronics (not shown), which are described elsewhere herein. In some examples, the core comprises a conductive material, such as titanium, stainless steel, tantalum, nitinol, a conductive polymer, and/or the like. However, in other examples, the core is formed from a non-conductive material, such as a non-conductive polymer. In yet other examples, the core comprises a plurality of layers of materials. For example, in examples the core includes an inner core and an outer core. In a further example, the inner core is formed of a first conductive material and the outer core is formed of a second conductive material. For example, in some examples, the first conductive material is stainless steel, titanium, tantalum, platinum, a platinum-iridium alloy, a conductive polymer, an alloy, and/or the like, and the second conductive material is conductive material selected to provide electrical conduction between the core and the first layer, and/or to attach the first layer to the core (e.g., if the first layer is formed of a material that does not attach well to the core material). In another example, the core is formed of a non-conductive material (e.g., a non-conductive metal and/or a non-conductive polymer) and the first layer is a conductive material, such as titanium, stainless steel, tantalum, nitinol, a conductive polymer, and/or the like. The core and the first layer can be of a single (or same) material, e.g., platinum. One skilled in the art appreciates that additional configurations are possible.

In some examples, the first layer is formed of a conductive material. The working electrode is an exposed portion of the surface of the first layer. Accordingly, the first layer is formed of a material configured to provide a suitable electroactive surface for the working electrode, a material such as but not limited to platinum, platinum-iridium, gold, palladium, iridium, nitinol, graphite, a ternary metal oxide composite, carbon, a conductive polymer, an alloy and/or the like.

In some example, second layer surrounds a least a portion of the first layer, thereby defining the boundaries of the working electrode. In some examples, the second layer serves as an insulator and is formed of an insulating material, such as polyimide, polyurethane, parylene, or any other known insulating materials, for example, fluorinated polymers, polyethylene terephthalate, polyurethane, polyimide, liquid crystal polymer, other nonconducting polymers, or the like. Glass or ceramic materials can also be employed. Other materials suitable for use include surface energy modified coating systems such as are marketed under the trade names AMC18, AMC148, AMC141, and AMC321 by Advanced Materials Components Express of Bellafonte, Pa. In some alternative examples, however, the working electrode does not require a coating of insulator.

In some examples, the second layer is disposed on the first layer and configured such that the working electrode is exposed via window. In another example, an elongated conductive body, including the core, the first layer and the second layer, is provided, and the working electrode is exposed (i.e., formed) by removing a portion of the second layer, thereby forming the window through which the electroactive surface of the working electrode (e.g., the exposed surface of the first layer) is exposed. In some examples, the working electrode is exposed by (e.g., window is formed by) removing a portion of the second and (optionally) third layers. Removal of coating materials from one or more layers of elongated conductive body (e.g., to expose the electroactive surface of the working electrode) can be performed by hand, excimer lasing, chemical etching, laser ablation, grit-blasting, or the like.

In some examples, the sensor further comprises a third layer comprising a conductive material. In further examples, the third layer comprises a reference electrode, which is formed of a silver-containing material that is applied onto the second layer (e.g., an insulator). The silver-containing material can include any of a variety of materials and be in various forms, such as, Ag/AgCl-polymer pastes, paints, polymer-based conducting mixture, and/or inks that are commercially available, for example. The third layer can be processed using a pasting/dipping/coating step, for example, using a die-metered dip coating process. In one exemplary example, an Ag/AgCl polymer paste is applied to an elongated body by dip-coating the body (e.g., using a meniscus coating technique) and then drawing the body through a die to meter the coating to a precise thickness. In some examples, multiple coating steps are used to build up the coating to a predetermined thickness.

In some examples, the silver grain in the Ag/AgCl solution or paste can have an average particle size corresponding to a maximum particle dimension that is less than about 100 microns, or less than about 50 microns, or less than about 30 microns, or less than about 20 microns, or less than about 10 microns, or less than about 5 microns. The silver chloride grain in the Ag/AgCl solution or paste can have an average particle size corresponding to a maximum particle dimension that is less than about 100 microns, or less than about 80 microns, or less than about 60 microns, or less than about 50 microns, or less than about 20 microns, or less than about 10 microns. The silver grain and the silver chloride grain can be incorporated at a ratio of the silver chloride grain:silver grain of from about 0.01:1 to 2:1 by weight, or from about 0.1:1 to 1:1. The silver grains and the silver chloride grains are then mixed with a carrier (e.g., a polyurethane) to form a solution or paste. In certain examples, the Ag/AgCl component form from about 10% to about 65% by weight of the total Ag/AgCl solution or paste, or from about 20% to about 50%, or from about 23% to about 37%. In some examples, the Ag/AgCl solution or paste has a viscosity (under ambient conditions) that is from about 1 to about 500 centipoise, or from about 10 to about 300 centipoise, of from about 50 to about 150 centipoise.

In examples, the above-exemplified sensor has an overall diameter of not more than about 0.20 inches (about 0.51 mm), more preferably not more than about 0.18 inches (about 0.46 mm), and most preferably not more than about 0.16 inches (0.41 mm). In some examples, the working electrode has a diameter of from about 0.001 inches or less to about 0.10 inches or more, preferably from about 0.002 inches to about 0.008 inches, and more preferably from about 0.004 inches to about 0.005 inches. The length of the window can be from about 0.1 mm (about 0.004 inches) or less to about 2 mm (about 0.78 inches) or more, and preferably from about 0.5 mm (about 0.2 inches) to about 0.75 mm (0.03 inches). In such examples, the exposed surface area of the working electrode is preferably from about 0.000013 in2 (0.0000839 cm2) or less to about 0.0025 in2(0.016129 cm2) or more (assuming a diameter of from about 0.001 inches to about 0.10 inches and a length of from about 0.004 inches to about 0.78 inches). The exposed surface area of the working electrode is selected to produce an analyte signal with a current in the femtoampere range, picoampere range, the nanoampere range, the or the microampere range such as is described in more detail elsewhere herein. However, a current in the picoampere range or less can be dependent upon a variety of factors, for example the electronic circuitry design (e.g., sample rate, current draw, A/D converter bit resolution, etc.), the membrane system (e.g., permeability of the analyte through the membrane system), and the exposed surface area of the working electrode. Accordingly, the exposed electroactive working electrode surface area can be selected to have a value greater than or less than the above-described ranges taking into consideration alterations in the membrane system and/or electronic circuitry. In examples of a glucose or lactate sensor, it can be advantageous to minimize the surface area of the working electrode while maximizing the diffusivity of glucose or lactate in order to optimize the signal-to-noise ratio while maintaining sensor performance in both high and low glucose or lactate concentration ranges.

In some alternative examples, the exposed surface area of the working (and/or other) electrode can be increased by altering the cross-section of the electrode itself. For example, in some examples the cross-section of the working electrode can be defined by a cross, star, cloverleaf, ribbed, dimpled, ridged, irregular, or other non-circular configuration; thus, for any predetermined length of electrode, a specific increased surface area can be achieved (as compared to the area achieved by a circular cross-section). Increasing the surface area of the working electrode can be advantageous in providing an increased signal responsive to the concentration of an analyte, which in turn can be helpful in improving the signal-to-noise ratio, for example.

In some examples, the elongated conductive body further comprises one or more intermediate layers located between the core and the first layer. For example, in some examples, the intermediate layer is an insulator, a conductor, a polymer, and/or an adhesive.

In certain example, the core comprises a non-conductive polymer and the first layer comprises a conductive material. Such a sensor configuration can sometimes provide reduced material costs, in that it replaces a typically expensive material with an inexpensive material. For example, in some examples, the core is formed of a non-conductive polymer, such as, a nylon or polyester filament, string or cord, which can be coated and/or plated with a conductive material, such as platinum, platinum-iridium, gold, palladium, iridium, graphite, carbon, a conductive polymer, and allows or combinations thereof.

In some examples, the sensor also includes a membrane covering at least a portion of the working electrode. Membranes are discussed in detail in greater detail elsewhere herein.

Exemplary sensor configurations can be applied to any planar or non-planar surface, for example. In another example, the sensor system has additional electrodes arranged as one or more concentric substantially ring-shaped electrodes, or rows or arrays of electrodes on a planar or substantially planar substrate.

As discussed herein, in some examples, the membrane system includes a bioprotective domain, also referred to as a cell-impermeable domain or biointerface domain, comprising a surface-modified base polymer as described in more detail elsewhere herein. In some examples, a unitary diffusion resistance domain and bioprotective domain can be included in the membrane system (e.g., wherein the functionality of both domains is incorporated into one domain, i.e., the bioprotective domain). In some examples, the sensor is configured for implantation from about 1 to 30 days). However, it is understood that the membrane system can be modified for use in other devices, for example, by including only one or more of the domains, or additional domains.

In some examples, the membrane system can include an electrode domain. The electrode domain is provided to ensure that an electrochemical reaction occurs between the electroactive surfaces of the working electrode and the reference electrode, and thus the electrode domain can be situated more proximal to the electroactive surfaces than the interference and/or enzyme domain. The electrode domain can include a coating that maintains a layer of water at the electrochemically reactive surfaces of the sensor. In other words, the electrode domain can be present to provide an environment between the surfaces of the working electrode and the reference electrode, which facilitates an electrochemical reaction between the electrodes.

A wide variety of configurations and combinations for the various layers in the membrane system are encompassed by the examples. In various examples, any of the domains described herein can be omitted, altered, substituted for, and/or incorporated together without departing from the spirit of the preferred examples. It is to be understood that sensing membranes modified for other sensors, for example, can include fewer or additional layers. For example, in some examples, the membrane system can comprise one electrode layer, one enzyme layer, and two bioprotective layers, but in other examples, the membrane system can comprise one electrode layer, two enzyme layers, and one bioprotective layer. In some examples, the bioprotective layer can be configured to function as the diffusion resistance domain and control the flux of the analyte (e.g., glucose, lactate, etc.) to the underlying membrane layers.

In some examples, a sensing membrane comprising one or more domains of polymeric membranes can be formed from materials such as polytetrafluoroethylene, silicone, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers.

In examples, a sensing membrane is disposed over the electroactive surfaces of the continuous transcutaneous analyte sensor and includes one or more domains or layers of a membrane system. In general, the sensing membrane functions to control the flux of a biological fluid there through and/or to protect sensitive regions of the sensor from contamination by the biological fluid, for example. Some conventional electrochemical enzyme-based analyte sensors generally include a sensing membrane that controls the flux of the analyte being measured, protects the electrodes from contamination of the biological fluid, and/or provides an enzyme that catalyzes the reaction of the analyte with a co-factor, for example. See, e.g., U.S. Patent Publication No. 2005-0245799A1 and U.S. Pat. No. 7,497,827, which are incorporated herein by reference in their entirety.

The sensing membranes of the present disclosure can include any membrane configuration suitable for use with any analyte sensor (such as described in more detail above). In general, the sensing membranes of the present disclosure include one or more domains, all or some of which can be adhered to or deposited on the analyte sensor as is appreciated by one skilled in the art. In examples, the sensing membrane generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface, such as described in the above-referenced U.S. patents and patent publications.

### Membrane Systems

In some examples, one or more domains of the membranes are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly(ethylene oxide), poly(propylene oxide) and copolymers and blends thereof, polysulfones and block copolymers thereof including, for example, di-block, tri-block, alternating, random and graft copolymers. U.S. Patent Publication No. 2005-0245799A1, which is incorporated herein by reference in its entirety, describes biointerface and sensing membrane configurations and materials that can be applied to the presently disclosed sensor.

### Electrode Domain

In some examples, the membrane system comprises an optional electrode domain. The electrode domain is provided to ensure that an electrochemical reaction occurs between the electroactive surfaces of the working electrode and the reference electrode, and thus the electrode domain is preferably situated more proximal to the electroactive surfaces than the enzyme domain. Preferably, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In examples, the electrode domain includes a flexible, water-swellable, hydrogel film having a “dry film” thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. “Dry film” thickness refers to the thickness of a cured film cast from a coating formulation by standard coating techniques.

In certain examples, the electrode domain is formed of a curable mixture of a urethane polymer and a hydrophilic polymer. Particularly preferred coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC))) in the presence of polyvinylpyrrolidone and cured at a moderate temperature of about 50° C.

Preferably, the electrode domain is deposited by spray or dip-coating the electroactive surfaces of the sensor. More preferably, the electrode domain is formed by dip-coating the electroactive surfaces in an electrode solution and curing the domain for a time of from about 15 to about 30 minutes at a temperature of from about 40 to about 55° C. (and can be accomplished under vacuum (e.g., 20 to 30 mmHg)). In examples wherein dip-coating is used to deposit the electrode domain, a preferred insertion rate of from about 1 to about 3 inches per minute, with a preferred dwell time of from about 0.5 to about 2 minutes, and a preferred withdrawal rate of from about 0.25 to about 2 inches per minute provide a functional coating. However, values outside of those set forth above can be acceptable or even desirable in certain examples, for example, dependent upon viscosity and surface tension as is appreciated by one skilled in the art. In examples, the electroactive surfaces of the electrode system are dip-coated one time (one layer) and cured at 50° C. under vacuum for 20 minutes.

Although an independent electrode domain is described herein, in some examples, sufficient hydrophilicity can be provided in the interference domain and/or enzyme domain (the domain adjacent to the electroactive surfaces) so as to provide for the full transport of ions in the aqueous environment (e.g. without a distinct electrode domain).

### Interference Domain

In some examples, an optional interference domain is provided, which generally includes a polymer domain that restricts the flow of one or more interferants. In some examples, the interference domain functions as a molecular sieve that allows analytes and other substances that are to be measured by the electrodes to pass through, while preventing passage of other substances, including interferants such as ascorbate and urea (see U.S. Pat. No. 6,001,67 to Shults). Some known interferants are caffeic acid, dopamine, L-tyrosine, 3-o-methyldopa, L-alpha-methyldopa, homocysteine, carbidopa, cresols (e.g., m-cresol, an insulin preservative), parabens (drug preservatives), and the like.

Several polymer types that can be utilized as a base material for the interference domain include polyurethanes, polymers having pendant ionic groups, and polymers having controlled pore size, for example. In some examples, the interference domain includes a thin, hydrophobic membrane that is non-swellable and restricts diffusion of low molecular weight species. The interference domain is permeable to relatively low molecular weight substances but restricts the passage of higher molecular weight substances. Other systems and methods for reducing or eliminating interference species that can be applied to the membrane system of the present disclosure are described in U.S. Pat. No. 7,816,004, U.S. Patent Publication No. 2005-0176136A1, U.S. Pat. No. 7,81,195, and U.S. Pat. No. 7,715,893. In some alternative examples, a distinct interference domain is not included.

In examples, the interference domain is deposited onto the electrode domain (or directly onto the electroactive surfaces when a distinct electrode domain is not included) for a domain thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. Unfortunately, the thin thickness of the interference domains conventionally used can introduce variability in the membrane system processing. For example, if too much or too little interference domain is incorporated within a membrane system, the performance of the membrane can be adversely affected.

### Enzyme Domain

In some examples, the membrane system further includes an enzyme domain disposed more distally from the electroactive surfaces than the interference domain (or electrode domain when a distinct interference is not included). In some examples, the enzyme domain is directly deposited onto the electroactive surfaces (when neither an electrode or interference domain is included). In other representative examples, the enzyme domain is deposited on the surface of an interference domain. In examples, the enzyme domain provides an enzyme to catalyze the reaction of the analyte and its co-reactant, as described in more detail below. Preferably, the enzyme domain includes polyphenol oxidase.

For an enzyme-based electrochemical glucose or lactate sensor to perform well, the sensor's response is preferably limited by neither enzyme activity nor co-reactant concentration. Because enzymes, including polyphenol oxidase, are subject to deactivation as a function of time even in ambient conditions, this behavior is compensated for in forming the enzyme domain. Preferably, the enzyme domain is constructed of aqueous dispersions of colloidal polyurethane polymers including the enzyme. However, in alternative examples the enzyme domain is constructed from materials with oxygen-enhancing performance, or high oxygen solubility, for example, silicone, or fluorocarbon, in order to provide a supply of excess oxygen during transient ischemia. Preferably, the enzyme is immobilized within the domain. See U.S. Pat. No. 7,379,765.

In examples, the enzyme domain is deposited onto the interference domain for a domain thickness of from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. However, in some examples, the enzyme domain is deposited onto the electrode domain or directly onto the electroactive surfaces. Preferably, the enzyme domain is deposited by spray or dip coating. More preferably, the enzyme domain is formed by dip-coating the electrode domain into an enzyme domain solution and curing the domain for from about 15 to about 30 minutes at a temperature of from about 40 to about 55° C. (and can be accomplished under vacuum (e.g., 20 to 30 mmHg)). In examples wherein dip-coating is used to deposit the enzyme domain at room temperature, a preferred insertion rate of from about 1 inch per minute to about 3 inches per minute, with a preferred dwell time of from about 0.5 minutes to about 2 minutes, and a preferred withdrawal rate of from about 0.25 inch per minute to about 2 inches per minute provide a functional coating. However, values outside of those set forth above can be acceptable or even desirable in certain examples, for example, dependent upon viscosity and surface tension as is appreciated by one skilled in the art. In examples, the enzyme domain is formed by dip coating two times (namely, forming two layers) in a coating solution and curing at 50° C. under vacuum for 20 minutes. However, in some examples, the enzyme domain can be formed by dip-coating and/or spray-coating one or more layers at a predetermined concentration of the coating solution, insertion rate, dwell time, withdrawal rate, and/or desired thickness.

### Resistance Domain

In examples, the membrane system includes a resistance domain disposed more distal from the electroactive surfaces than the enzyme domain. Although the following description is directed to a resistance domain for a glucose and/or lactate sensor, the resistance domain can be modified for other analytes and co-reactants as well.

The resistance domain includes a semi-permeable membrane that controls the flux of lactate to the underlying enzyme domain, preferably rendering oxygen in a non-rate-limiting excess. As a result, the upper limit of linearity of lactate measurement is extended to a much higher value than that which is achieved without the resistance domain. In examples, the resistance domain exhibits an oxygen to lactate permeability ratio such that one-dimensional reactant diffusion is adequate to provide excess oxygen at all reasonable lactate and oxygen concentrations found in the subcutaneous matrix.

In alternative examples, a lower ratio of oxygen-to-lactate can be sufficient to provide excess oxygen by using a high oxygen solubility domain (for example, a silicone or fluorocarbon-based material or domain) to enhance the supply/transport of oxygen to the enzyme domain. If more oxygen is supplied to the enzyme, then more lactate can also be supplied to the enzyme without creating an oxygen rate-limiting excess. In alternative examples, the resistance domain is formed from a silicone composition, such as is described in U.S. Patent Publication No. US 2005/0090607 filed Oct. 28, 2003 and entitled, “SILICONE COMPOSITION FOR BIOCOMPATIBLE MEMBRANE.”

In some examples, the presently disclosed continuous lactate monitoring (CLM) sensor includes a resistance domain to control the diffusion of lactate and oxygen to the CLM sensor, fabricated easily and reproducibly from commercially available materials. A suitable resistance domain component is a polyurethane or polyurethaneurea (hereinafter, collectively referred to as “PU”) which can be a thermoplastic polyurethane or polyurethaneurea or blend thereof. Polyurethane is a polymer produced by the condensation reaction of a diisocyanate and a difunctional hydroxyl-containing material. A polyurethaneurea is a polymer produced by the condensation reaction of a diisocyanate and a difunctional amine-containing material. Exemplary diisocyanates include aliphatic diisocyanates containing from about 4 to about 8 methylene units. Diisocyanates containing cycloaliphatic moieties can also be useful in the preparation of the polymer and copolymer components of the membranes of the present disclosure.

In some examples, a PU polymer is provided with a hard segment and a soft segment, where the soft segment comprises two or more polycarbonate segments, polydimethylsiloxane segments, and polyalkyene oxide segments. In examples, a PU polymer is provided with a hard segment of about 35-45 weight percent, and a soft segment (remainder weight percent+up to 10 weight percent chain extender), where the soft segment comprises two or more polycarbonate segments, polydimethylsiloxane segments, and polyalkyene oxide segments. In examples, the soft segment comprises 35-45 weight percent polycarbonate segments and 15-20 weight percent polydimethylsiloxane segments, the remainder weight percent being hard segment and chain extender. In other examples, the soft segment comprises 35-45 weight percent polyakylene segments and 15-20 weight percent polydimethylsiloxane segments the remainder weight percent being hard segment and chain extender. In other examples, the soft segment comprises 35-45 weight percent total of both polyakylene segments and polycarbonate segments, and 15-20 weight percent polydimethylsiloxane segments the remainder weight percent being hard segment and chain extender. In examples, the polyalkylene segment comprises poly(tetramethylene oxide) (PTMO). In examples, PU polymer is provided with a hard segment and a soft segment, where the soft segment comprises two or more polycarbonate segments, polydimethylsiloxane segments, and polyalkyene oxide segments blended with a polyvinylpyrrolidone (PVP).

In some examples, a diffusion resistance layer (RL) of the presently disclosed CFM includes the aforementioned PU polymer and/or PU polymer-PVP blend that provides stable, predicable lactate and oxygen permeation and blocks at least some interfering agents. It will be appreciated that the hard/soft segment chemical composition, weight percentage of hard/soft segment, topology and block length distribution will impact the RL phase separation, hard segment/soft segment interaction, lactate permeability, solubility of RL formulation for coating/dispensing and drying/curing processes and thus, influence sensor performance and stability.

In other examples, materials that forms the basis of the matrix of the resistance domain can be any of those known in the art as appropriate for use as membranes in sensor devices and as having sufficient permeability to allow relevant compounds to pass through it, for example, to allow lactate to pass through the membrane from the sample under examination in order to reach the active enzyme or electrochemical electrodes. Examples of materials which can be used to make non-polyurethane type membranes include vinyl polymers, polyethers, polyesters, polyamides, inorganic polymers such as polysiloxanes and polycarbosiloxanes, natural polymers such as cellulosic and protein-based materials, poly(vinyl alcohol)-quaternized stilbazol (PVA-SbQ), and mixtures or combinations thereof.

In some examples, the resistance domain is deposited onto the enzyme domain to yield a domain thickness from about 0.5 micron or less to about 20 microns or more, more preferably from about 0.5, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. Preferably, the resistance domain is deposited onto the enzyme domain by spray coating or dip coating. In certain examples, spray coating is the preferred deposition technique. The spraying process atomizes and mists the solution, and therefore most or all of the solvent is evaporated prior to the coating material settling on the underlying domain, thereby minimizing contact of the solvent with the enzyme.

In examples, the resistance domain is deposited on the enzyme domain by spray-coating a solution of from about 1 wt. % to about 5 wt. % polymer and from about 95 wt. % to about 99 wt. % solvent. In spraying a solution of resistance domain material, including a solvent, onto the enzyme domain, it is desirable to mitigate or substantially reduce any contact with enzyme of any solvent in the spray solution that can deactivate the underlying enzyme of the enzyme domain. Tetrahydrofuran (THF) is one solvent that minimally or negligibly affects the enzyme of the enzyme domain upon spraying. Other solvents can also be suitable for use, as is appreciated by one skilled in the art.

Although a variety of spraying or deposition techniques can be used, spraying the resistance domain material and rotating the sensor at least one time by 180° can provide adequate coverage by the resistance domain. Spraying the resistance domain material and rotating the sensor at least two times by 120 degrees provides even greater coverage (one layer of 360° coverage), thereby ensuring resistivity to lactate, such as is described in more detail above.

In examples, the resistance domain is spray-coated and subsequently cured for a time of from about 15 to about 90 minutes at a temperature of from about 40 to about 60° C. (and can be accomplished under vacuum (e.g., 20 to 30 mmHg)). A cure time of up to about 90 minutes or more can be advantageous to ensure complete drying of the resistance domain. While not wishing to be bound by theory, it is believed that complete drying of the resistance domain aids in stabilizing the sensitivity of the lactate sensor signal. It reduces drifting of the signal sensitivity over time, and complete drying is believed to stabilize performance of the lactate sensor signal in lower oxygen environments.

In examples, a sensor signal with a current in the picoampere range or less is provided, which is described in more detail elsewhere herein. However, the ability to produce a signal with a current in the picoampere range can be dependent upon a combination of factors, including the electronic circuitry design (e.g., A/D converter, bit resolution, and the like), the membrane system (e.g., permeability of the analyte through the resistance domain, enzyme concentration, and/or electrolyte availability to the electrochemical reaction at the electrodes), and the exposed surface area of the working electrode. For example, the resistance domain can be designed to be more or less restrictive to the analyte depending upon to the design of the electronic circuitry, membrane system, and/or exposed electroactive surface area of the working electrode.

### Interference-Free Membrane Systems

In general, it is believed that appropriate solvents and/or deposition methods can be chosen for one or more of the domains of the membrane system that form one or more transitional domains such that interferants do not substantially permeate there through. Thus, sensors can be built without distinct or deposited interference domains, which are non-responsive to interferants. While not wishing to be bound by theory, it is believed that a simplified multilayer membrane system, more robust multilayer manufacturing process, and reduced variability caused by the thickness and associated oxygen and lactate and/or glucose sensitivity of the deposited micron-thin interference domain can be provided.

### Biointerface Membrane/Layer

In examples, the sensor includes a porous material disposed over some portion thereof, which modifies the host's tissue response to the sensor. In some examples, the porous material surrounding the sensor advantageously enhances and extends sensor performance and lifetime by slowing or reducing cellular migration to the sensor and associated degradation that would otherwise be caused by cellular invasion if the sensor were directly exposed to the in vivo environment. Alternatively, the porous material can provide stabilization of the sensor via tissue ingrowth into the porous material in the long term. Suitable porous materials include silicone, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polybutylene terephthalate (PBT), polymethylmethacrylate (PMMA), polyether ether ketone (PEEK), polyamides, polyurethanes, cellulosic polymers, poly(ethylene oxide), poly(propylene oxide) and copolymers and blends thereof, polysulfones and block copolymers thereof including, for example, di-block, tri-block, alternating, random and graft copolymers, as well as metals, ceramics, cellulose, hydrogel polymers, poly(2-hydroxyethyl methacrylate, pHEMA), hydroxyethyl methacrylate, (HEMA), polyacrylonitrile-polyvinyl chloride (PAN-PVC), high density polyethylene, acrylic copolymers, nylon, polyvinyl difluoride, polyanhydrides, poly(l-lysine), poly(L-lactic acid), hydroxyethylmethacrylate, hydroxyapeptite, alumina, zirconia, carbon fiber, aluminum, calcium phosphate, titanium, titanium alloy, nitinol, stainless steel, and CoCr alloy, or the like, such as are described in U.S. Pat. Nos. 7,875,293 and 7,192,450.

In some examples, the porous material surrounding the sensor provides unique advantages in vivo (e.g., one to 14 days) that can be used to enhance and extend sensor performance and lifetime. However, such materials can also provide advantages in the long term too (e.g., greater than 14 days). Particularly, the in vivo portion of the sensor (the portion of the sensor that is implanted into the host's tissue) is encased (partially or fully) in a porous material. The porous material can be wrapped around the sensor (for example, by wrapping the porous material around the sensor or by inserting the sensor into a section of porous material sized to receive the sensor). Alternately, the porous material can be deposited on the sensor (for example, by electrospinning of a polymer directly thereon). In yet other alternative examples, the sensor is inserted into a selected section of porous biomaterial. Other methods for surrounding the in vivo portion of the sensor with a porous material can also be used as is appreciated by one skilled in the art.

The porous material surrounding the sensor advantageously slows or reduces cellular migration to the sensor and associated degradation that would otherwise be caused by cellular invasion if the sensor were directly exposed to the in vivo environment. Namely, the porous material provides a barrier that makes the migration of cells towards the sensor more tortuous and therefore slower. It is believed that this reduces or slows the sensitivity loss normally observed over time.

In examples wherein the porous material is a high oxygen solubility material, such as porous silicone, the high oxygen solubility porous material surrounds some of or the entire in vivo portion of the sensor. In some examples, a lower ratio of oxygen-to-lactate can be sufficient to provide excess oxygen by using a high oxygen soluble domain (for example, a silicone- or fluorocarbon-based material) to enhance the supply/transport of oxygen to the enzyme membrane and/or electroactive surfaces. It is believed that some signal noise normally seen by a conventional sensor can be attributed to an oxygen deficit. Silicone has high oxygen permeability, thus promoting oxygen transport to the enzyme layer. By enhancing the oxygen supply through the use of a silicone composition, for example, lactate concentration can be less of a limiting factor. In other words, if more oxygen is supplied to the enzyme and/or electroactive surfaces, then more lactate can also be supplied to the enzyme without creating an oxygen rate-limiting excess. While not being bound by any particular theory, it is believed that silicone materials provide enhanced bio-stability when compared to other polymeric materials such as polyurethane.

In another example, the porous material further comprises a bioactive agent that releases upon insertion. In examples, the porous structure provides access for lactate permeation while allowing drug release/elute. In examples, as the bioactive agent releases/elutes from the porous structure, lactate transport can increase, for example, so as to offset any attenuation of lactate transport from the aforementioned immune response factors.

When used herein, the terms “membrane” and “matrix” are meant to be interchangeable. In these examples, the aforementioned porous material is a biointerface membrane comprising a first domain that includes an architecture, including cavity size, configuration, and/or overall thickness, that modifies the host's tissue response, for example, by creating a fluid pocket, encouraging vascularized tissue ingrowth, disrupting downward tissue contracture, resisting fibrous tissue growth adjacent to the device, and/or discouraging barrier cell formation. The biointerface membrane in examples covers at least the sensing mechanism of the sensor and can be of any shape or size, including uniform, asymmetrically, or axi-symmetrically covering or surrounding a sensing mechanism or sensor.

A second domain of the biointerface membrane is optionally provided that is impermeable to cells and/or cell processes. A bioactive agent is optionally provided that is incorporated into the at least one of the first domain, the second domain, the sensing membrane, or other part of the implantable device, wherein the bioactive agent is configured to modify a host tissue response. In examples, the biointerface includes a bioactive agent, the bioactive agent being incorporated into at least one of the first and second domains of the biointerface membrane, or into the device and adapted to diffuse through the first and/or second domains, in order to modify the tissue response of the host to the membrane.

Due to the small dimension(s) of the sensor (sensing mechanism) of the present disclosure, some conventional methods of porous membrane formation and/or porous membrane adhesion are inappropriate for the formation of the biointerface membrane onto the sensor as described herein. Accordingly, the following examples exemplify systems and methods for forming and/or adhering a biointerface membrane onto a small structured sensor as defined herein. For example, the biointerface membrane or release membrane of the present disclosure can be formed onto the sensor using techniques such as electrospinning, molding, weaving, direct-writing, lyophilizing, wrapping, and the like.

In examples wherein the biointerface is directly-written onto the sensor, a dispenser dispenses a polymer solution using a nozzle with a valve, or the like, for example as described in U.S. Publication No. 2004/0253365 A1. In general, a variety of nozzles and/or dispensers can be used to dispense a polymeric material to form the woven or non-woven fibers of the biointerface membrane.

### Definitions

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases can refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7,10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein can refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein can refer to two or more system elements or components that are configured to be or have been electrically, mechanically, thermally, operably, chemically, or otherwise attached and detached without damaging any of the coupled elements or components. The phrase “permanently coupled” as used herein can refer to two or more system elements or components that are configured to be or have been electrically, mechanically, thermally, operably, chemically, or otherwise attached but cannot be uncoupled without damaging at least one of the coupled elements or components. covalently, electrostatically, ionically associated with, or physically entrapped, or absorbed.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

### EXAMPLE EMBODIMENTS

Implementation examples are described in the following numbered clauses:
Clause 1: A monitoring system, comprising: one or more memories comprising executable instructions; and one or more processors in data communication with the one or more memories and configured to execute the executable instructions to: calculate a gastric emptying rate of a patient based on glucose measurements and lactate measurements; determine whether the gastric emptying rate of the patient is decreasing; determine, if the gastric emptying rate of the patient is decreasing, whether the gastric emptying rate of the patient meets a first threshold, or whether a reduction in the gastric emptying rate over a defined period of time meets a second threshold; provide therapy management action to the patient based on the determined gastric emptying rate of the patient to optimize the gastric emptying rate of the patient; and recalculate, following the therapy management action, the gastric emptying rate of the patient based on the glucose measurements and the lactate measurements.
Clause 2: A monitoring system, comprising: one or more memories comprising executable instructions; and one or more processors in data communication with the one or more memories and configured to execute the executable instructions to: calculate a gastric emptying rate of the patient based on the glucose measurements and the lactate measurements.
Clause 3: The monitoring system of Clause 2, wherein the one or more processors are further configured to automatically alter a GLP-1 administration regimen based on (1) a determination of whether the gastric emptying rate of the patient is decreasing and (2) a determination of whether the gastric emptying rate of the patient meets a first threshold, or whether a reduction in the gastric emptying rate over a defined period of time meets a second threshold in accordance with the monitoring.
Clause 4: A monitoring system, comprising: one or more memories comprising executable instructions; and one or more processors in data communication with the one or more memories and configured to execute the executable instructions to: monitor glucose measurements, lactate measurements, and a GLP-1 dose of the patient; calculate an expected weight loss of the patient based on the glucose measurements, the lactate measurements, and the GLP-1 dose; determine whether the expected weight loss of the patient is within a threshold of an expected weight loss; determine whether the patient has reached a weight loss goal, wherein if the weight loss of the patient is within the threshold of the expected weight loss based on the historical patient population and the patient has reached the weight loss goal, provide therapy management action to the patient to reduce the GLP-1 dose of the patient; and monitor, following the therapy management action, the glucose measurements, the lactate measurements, and the GLP-1 dose of the patient.
Clause 5: A monitoring system, comprising: one or more memories comprising executable instructions; and one or more processors in data communication with the one or more memories and configured to execute the executable instructions to: monitor glucose measurements, lactate measurements, and a GLP-1 regimen of the patient.
Clause 6: The monitoring system of Clause 5, wherein the one or more processors are further configured to automatically alter a GLP-1 administration regimen based on (1) a calculation of an expected weight loss of the patient based on the monitoring and (2) a determination of whether the patient has reached a weight loss goal.
Clause 7: The monitoring system of any one of Clauses I-6, wherein the monitoring system further comprises: a continuous analyte sensor configured to penetrate a skin of a patient and generate sensor current indicative of analyte levels of the patient; a sensor electronics module coupled to the continuous analyte sensor, wherein the sensor electronics module comprises: an analog to digital converter configured to: receive the sensor current; and convert the sensor current generated by the continuous analyte sensor into digital signals; one or more processors configured to convert the digital signals to a set of analyte measurements indicative of the analyte levels of the patient; and a Bluetooth antenna configured to transmit the set of analyte measurements wirelessly to a wireless communications device using Bluetooth or BLE communications protocols.
Clause 8: The monitoring system of any one of Clauses 1-7, wherein the sensor electronic module further comprises a sensitivity profile for the monitoring system based on a calibration process performed during manufacturing, wherein one or more processors being configured to convert the digital signals to the set of analyte measurements comprises converting the digital signals to the set of analyte measurements based on the sensitivity profile.
Clause 9: The monitoring system of any one of Clauses 1-8, wherein the continuous analyte sensor comprises: a percutaneous wire comprising: a proximal portion coupled to the sensor electronics module; and a distal portion comprising a working electrode and a reference electrode, wherein the working electrode is configured to penetrate the skin and extend into a dermis or subcutaneous tissue of the patient.
Clause 10: The monitoring system of any one of Clauses 1-9, wherein the working electrode and the reference electrodes are disposed on a substrate, and the sensor current is at least in part based on a voltage difference generated between the working electrode and the reference electrode.
Clause 11: The monitoring system of any one of Clauses 1-10, wherein the continuous analyte sensor is a multi-analyte sensor comprising a continuous glucose sensor and a continuous lactate sensor, and the set of analyte measurements include glucose measurements and lactate measurements.
Clause 12: The monitoring system of any one of Clauses 1-11, further comprising: one or more memories comprising executable instructions; and one or more processors in data communication with the one or more memories and configured to execute the executable instructions to: calculate a gastric emptying rate of the patient based on the glucose measurements and the lactate measurements.
Clause 13: The monitoring system of any one of Clauses 1-3 or 7-12, wherein, based on a determination that the gastric emptying rate of the patient is decreasing and a determination that the gastric emptying rate of the patient meets the first threshold, or that the reduction in the gastric emptying rate over the defined period of time meets the second threshold, the one or more processors being configured to automatically alter the GLP-1 administration regimen comprises the one or more processors being configured to automatically alter the GLP-1 administration regimen by decreasing a GLP-1 dose.
Clause 14: The monitoring system of any one of Clauses 1-3 or 7-13, wherein, based on a determination that the gastric emptying rate of the patient is not decreasing, the one or more processors are further configured to determine whether the patient is experiencing weight loss.
Clause 15: The monitoring system of any one of Clauses 1-3 or 7-14, wherein the one or more processors being configured to automatically alter the GLP-1 administration regimen comprises the one or more processors being configured to automatically alter the GLP-1 administration regimen by increasing the GLP-1 dose to decrease the gastric emptying rate of the patient based on a determination that the patient is not experiencing weight loss.
Clause 16: The monitoring system of any one of Clauses 1-3 or 7-15, wherein the determination of whether the gastric emptying rate of the patient is decreasing is based on the glucose measurements or the lactate measurements in response to consumption of a meal.
Clause 17: The monitoring system of any one of Clauses 1-3 or 7-16, wherein the determination of whether the gastric emptying rate of the patient meets the first threshold is based on (1) a GLP-1 regimen of the patient and (2) a gastric emptying rate of a patient population prescribed a similar GLP-1 regimen to the patient.
Clause 18: The monitoring system of any one of Clauses 1-3 or 7-17, wherein automatically altering the GLP-1 administration regimen comprises increasing the GLP-1 dose of the patient, decreasing the GLP-1 dose of the patient, altering a type of GLP-1 of the patient, or recommending a time of administration of the GLP-1 dose.
Clause 19: The monitoring system of any one of Clauses 1-3 or 7-18, wherein the one or more processors are further configured to, following the automatically altering of the GLP-1 administration regimen, recalculate the gastric emptying rate of the patient based on the glucose measurements and the lactate measurements.
Clause 20: The monitoring system of any one of Clauses 1-3 or 7-19, wherein, based on a determination that the gastric emptying rate of the patient does not meet the first threshold or a determination that the reduction in the gastric emptying rate over the defined period of time does not meet the second threshold, the one or more processors are further configured to determine whether the patient is experiencing digestive symptoms.
Clause 21: The monitoring system of any one of Clauses 1-3 or 7-20, wherein the one or more processors are further configured to determine a severity of the digestive symptoms of the patient.
Clause 22: The monitoring system of any one of Clauses 1-3 or 7-21, wherein, based on the severity of the digestive symptoms of the patient, the one or more processors are further configured to provide the altered GLP-1 administration regimen to the patient to manage or address the digestive symptoms.
Clause 23: The monitoring system of any one of Clauses 1-3 or 7-22, wherein, based on a determination that the severity of the digestive symptoms are below a threshold, the one or more processors are further configured to determine if the digestive symptoms of the patient resolve over time.
Clause 24: The monitoring system of any one of Clauses 1-3 or 7-23, wherein, based on a determination that the digestive symptoms of the patient have not resolved over time, the one or more processors are further configured to provide a recommendation to the patient to alter at least one of a diet of the patient, an exercise timing, or a meal timing.
Clause 25: The monitoring system of any one of Clauses 1-3 or 7-24, wherein, based on a determination that the severity of the digestive symptoms are above a threshold, the one or more processors are further configured to provide a recommendation to the patient to seek medical intervention for an intestinal blockage.
Clause 26: The monitoring system of any one of Clauses 4-6, wherein the calculation of the expected weight loss of the patient is based on a glucose time in range, a timing of a peak of a glucose level, a magnitude of the peak of the glucose level, a rate of change of increase of glucose levels, a rate of change of decrease of glucose levels, or a duration of glucose level increase following consumption of a meal.
Clause 27: The monitoring system of any one of Clauses 4-12 or 26, wherein the determination of whether the expected weight loss of the patient is within the threshold of the expected weight loss is based on an expected weight loss of a historical patient population prescribed a similar GLP-1 dose.
Clause 28: The monitoring system of any one of Clauses 4-12 or 26-27, wherein, based on a determination that the expected weight loss of the patient is not within the threshold of the expected weight loss in accordance with the historical patient population prescribed the similar GLP-1 dose, the one or more processors are further configured to determine whether the patient is experiencing severe gastrointestinal symptoms.
Clause 29: The monitoring system of any one of Clauses 4-12 or 26-28, wherein, based on a determination that the severity of the digestive symptoms is above the threshold, the one or more processors are further configured to provide a recommendation to seek medical intervention for an intestinal blockage.
Clause 30: The monitoring system of any one of Clauses 4-12 or 26-29, wherein, based on a determination that the severity of the digestive symptoms is below the threshold, the one or more processors are further configured to determine if the gastrointestinal symptoms of the patient resolve over time.
Clause 31: The monitoring system of any one of Clauses 4-12 or 26-30, wherein, based on a determination that the expected weight loss of the patient is within the threshold of the expected weight loss in accordance with the historical patient population prescribed the similar GLP-1 dose, and a determination that the patient reached the weight loss goal, the one or more processors being configured to automatically alter the GLP-1 administration regimen comprises the one or more processors being configured to automatically alter the GLP-1 administration regimen by decreasing or discontinuing the GLP-1 dose.
Clause 32: The monitoring system of any one of Clauses 4-12 or 26-31, wherein upon decreasing the GLP-1 dose, the one or more processors are further configured to continuously monitor the glucose measurements, lactate measurements, and the weight loss of the patient to determine whether the patient is maintaining weight loss.
Clause 33: The monitoring system of any one of Clauses 4-12 or 26-32, wherein, based on a determination that the patient is maintaining weight loss, the one or more processors being configured to automatically alter the GLP-1 administration regimen comprises the one or more processors being configured to automatically alter the GLP-1 administration regimen by further decreasing or discontinuing the GLP-1 dose.
Clause 34: The monitoring system of any one of Clauses 4-12 or 26-33, wherein, based on a determination that the patient is not maintaining weight loss, the one or more processors are further configured to provide a recommendation to the patient to alter at least one of an exercise schedule of the patient or a diet of the patient.
Clause 35: The monitoring system of any one of Clauses 4-12 or 26-34, wherein, based on the determination that the patient is maintaining weight loss and upon discontinuing the GLP-1 dose, the one or more processors are further configured to determine if the patient is maintaining weight loss over time and continue monitoring the glucose measurements and the lactate measurements of the patient.
Clause 36: The monitoring system of any one of Clauses 4-12 or 26-35, wherein the GLP-1 dose of the patient is determined based on GLP-1 medication adherence information or based on the glucose measurements and the lactate measurements.
Clause 37: The monitoring system of any one of Clauses 4-12 or 26-36, wherein the one or more processors are further configured to, following the altering of the GLP-1 administration regimen, continue monitoring the glucose measurements, lactate measurements, and the GLP-1 dose.
Clause 38: A method for performing operations performed by the system in Clauses 1-37.
Clause 39: A device configured to perform operations performed by the system in Clauses 1-37.

### ADDITIONAL CONSIDERATIONS

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to penetrate a skin of a patient and generate sensor current indicative of analyte levels of the patient;
a sensor electronics module coupled to the continuous analyte sensor, wherein the sensor electronics module comprises:
an analog to digital converter configured to:
receive the sensor current; and
convert the sensor current generated by the continuous analyte sensor into digital signals;
one or more processors configured to convert the digital signals to a set of analyte measurements indicative of the analyte levels of the patient; and
a Bluetooth antenna configured to transmit the set of analyte measurements wirelessly to a wireless communications device using Bluetooth or BLE communications protocols.

2. The monitoring system of claim 1, wherein the sensor electronic module further comprises a sensitivity profile for the monitoring system based on a calibration process performed during manufacturing, wherein one or more processors being configured to convert the digital signals to the set of analyte measurements comprises converting the digital signals to the set of analyte measurements based on the sensitivity profile.

3. The monitoring system of claim 1, wherein the continuous analyte sensor comprises:
a percutaneous wire comprising:
a proximal portion coupled to the sensor electronics module; and
a distal portion comprising a working electrode and a reference electrode, wherein the working electrode is configured to penetrate the skin and extend into a dermis or subcutaneous tissue of the patient.

4. The monitoring system of claim 3, wherein:
the working electrode and the reference electrodes are disposed on a substrate, and
the sensor current is at least in part based on a voltage difference generated between the working electrode and the reference electrode.

5. The monitoring system of claim 1, wherein:
the continuous analyte sensor is a multi-analyte sensor comprising a continuous glucose sensor and a continuous lactate sensor, and
the set of analyte measurements include glucose measurements and lactate measurements.

6. The monitoring system of claim 5, further comprising:
one or more memories comprising executable instructions; and
one or more processors in data communication with the one or more memories and configured to execute the executable instructions to:
calculate a gastric emptying rate of the patient based on the glucose measurements and the lactate measurements.

7. The monitoring system of claim 6, wherein the one or more processors are further configured to automatically alter a GLP-1 administration regimen based on (1) a determination of whether the gastric emptying rate of the patient is decreasing and (2) a determination of whether the gastric emptying rate of the patient meets a first threshold, or whether a reduction in the gastric emptying rate over a defined period of time meets a second threshold in accordance with the monitoring.

8. The monitoring system of claim 7, wherein, based on a determination that the gastric emptying rate of the patient is decreasing and a determination that the gastric emptying rate of the patient meets the first threshold, or that the reduction in the gastric emptying rate over the defined period of time meets the second threshold, the one or more processors being configured to automatically alter the GLP-1 administration regimen comprises the one or more processors being configured to automatically alter the GLP-1 administration regimen by decreasing a GLP-1 dose.

9. The monitoring system of claim 7, wherein, based on a determination that the gastric emptying rate of the patient is not decreasing, the one or more processors are further configured to determine whether the patient is experiencing weight loss.

10. The monitoring system of claim 9, wherein the one or more processors being configured to automatically alter the GLP-1 administration regimen comprises the one or more processors being configured to automatically alter the GLP-1 administration regimen by increasing a GLP-1 dose to decrease the gastric emptying rate of the patient based on a determination that the patient is not experiencing weight loss.

11. The monitoring system of claim 7, wherein the determination of whether the gastric emptying rate of the patient is decreasing is based on the glucose measurements or the lactate measurements in response to consumption of a meal.

12. The monitoring system of claim 7, wherein the determination whether the gastric emptying rate of the patient meets the first threshold is based on (1) a GLP-1 regimen of the patient and (2) a gastric emptying rate of a patient population prescribed a similar GLP-1 regimen to the patient.

13. The monitoring system of claim 7, wherein automatically altering the GLP-1 administration regimen comprises increasing a GLP-1 dose of the patient, decreasing the GLP-1 dose of the patient, altering a type of GLP-1 of the patient, or recommending a time of administration of the GLP-1 dose.

14. The monitoring system of claim 7, wherein the one or more processors are further configured to, following the automatically altering of the GLP-1 administration regimen, recalculate the gastric emptying rate of the patient based on the glucose measurements and the lactate measurements.

15. The monitoring system of claim 7, wherein, based on a determination that the gastric emptying rate of the patient does not meet the first threshold and a determination that the reduction in the gastric emptying rate over the defined period of time does not meet the second threshold, the one or more processors are further configured to determine whether the patient is experiencing digestive symptoms.

16. The monitoring system of claim 15, wherein the one or more processors are further configured to determine a severity of the digestive symptoms of the patient.

17. The monitoring system of claim 16, wherein, based on the severity of the digestive symptoms of the patient, the one or more processors are further configured to provide an altered GLP-1 administration regimen to the patient to manage or address the digestive symptoms.

18. The monitoring system of claim 17, wherein, based on a determination that the severity of the digestive symptoms are below a threshold, the one or more processors are further configured to determine if the digestive symptoms of the patient resolve over time.

19. The monitoring system of claim 18, wherein, based on a determination that the digestive symptoms of the patient have not resolved over time, the one or more processors are further configured to provide a recommendation to the patient to alter at least one of a diet of the patient, an exercise timing, or a meal timing.

20. The monitoring system of claim 17, wherein, based on a determination that the severity of the digestive symptoms are above a threshold, the one or more processors are further configured to provide a recommendation to the patient to seek medical intervention for an intestinal blockage.
